(19) United States
(12) Patent Application Publication
(10) Pub. No.: US 2024/0315724 A1
(43) Pub. Date: Sep. 26, 2024

(54) TISSUE TREATMENT SYSTEM HAVING CONTRAST INJECTOR

(71) Applicant: Otsuka Medical Devices Co., Ltd., Tokyo (JP)

(72) Inventors: James D. Mazzone, San Jose, CA (US); David Lawrence, Mountain View, CA (US); Luis Bustamante, San Jose, CA (US); John R. Stetak, III, San Jose, CA (US)

(21) Appl. No.: 18/614,170

(22) Filed: Mar. 22, 2024

Related U.S. Application Data
(60) Provisional application No. 63/492,192, filed on Mar. 24, 2023.
(60) Provisional application No. 63/501,364, filed on May 10, 2023.

(51) Int. Cl. A61B 17/32; A61B 90/00; A61M 25/10

(52) U.S. Cl. CPC A61B 17/320068; A61B 90/39; A61B 2017/320069; A61B 2090/0472; A61B 2090/3933; A61M 2025/1079

(57) ABSTRACT

A method of controlling injection of a contrast medium by a contrast injector includes delivering, by a fluid transfer unit of a tissue treatment system, inflation fluid at an inflation pressure to a balloon. The method includes injecting, by a contrast injector of the tissue treatment system, contrast medium at an injection pressure into a vessel containing the balloon. The method includes determining one or more of the inflation pressure or the injection pressure. The method includes stopping, based on one or more of the inflation pressure or the injection pressure, injection of the contrast medium. Other embodiments are also described and claimed.

This application claims the benefit of priority of U.S. Provisional Patent Application No. 63/492,192, filed on Mar. 24, 2023, titled “TISSUE TREATMENT SYSTEM HAVING CONTRAST INJECTOR,” and U.S. Provisional Patent Application No. 63/501,364, filed on May 10, 2023, titled “TREATMENT SYSTEM HAVING GENERATOR AND FLUID TRANSFER CARTRIDGE,” which are incorporated herein by reference in their entirety to provide continuity of disclosure.

### BACKGROUND

### Field

This application relates generally to medical apparatuses, systems, and methods that deliver energy and fluid to a device used to target an anatomical location of a subject. More specifically, this application relates to a contrast flow injector integrated to the apparatuses, systems, and methods for the treatment of tissue, such as nerve tissue. This application also relates to a fluid transfer cartridge to deliver cooling fluid to a catheter-based intraluminal device.

### Background Information

High blood pressure, also known as hypertension, commonly affects adults. Left untreated, hypertension can result in renal disease, arrhythmias, and heart failure. In recent years, the treatment of hypertension has focused on interventional approaches to inactivate the renal nerves surrounding a renal artery. Autonomic nerves tend to follow blood vessels to the organs that they innervate. Intraluminal devices, such as catheters, may reach specific structures, such as the renal nerves, which are proximate to the lumens in which the catheters travel. Accordingly, catheter-based systems can deliver energy from within the lumens to inactivate the renal nerves in the vessel walls.

One approach to renal nerve deactivation uses radio frequency (RF) energy. The RF energy is delivered to a catheter having multiple electrodes placed against the intima of the renal artery to create an electrical field in the vessel wall and surrounding tissue. The electrical field results in resistive (ohmic) heating of the tissue to ablate the tissue and the renal nerve passing through that tissue. To treat all the renal nerves surrounding the renal arteries, the RF electrodes are repositioned several times around the inside of the renal artery.

Another approach to renal nerve deactivation uses high-intensity focused ultrasound (HIFU). HIFU relies on the delivery of vibrational energy to a catheter to cause frictional heating and disruption of tissue. In turn, a temperature of the tissue elevates sufficiently to cause ablation or remodeling of the tissue containing the renal nerves. However, the use of HIFU intravascularly may result in, at most, the formation of a thin focal ring in the vessel and surrounding tissue. If applied to renal denervation, it would be difficult to align this thin ring with the renal nerves because the renal nerves lie at differing radial distances along the length of the renal arteries. Also problematic is that the thin focal ring results in a small longitudinal treatment zone relative to the axis of the vessel.

Many of the problems associated with RF and HIFU systems are solved by a system having an ultrasound transducer that emits one or more therapeutic doses of unfocused ultrasound energy. The ultrasound transducer can be mounted at a distal end of catheter, and the unfocused ultrasound energy can heat tissue adjacent to a body lumen within which the catheter (and the transducer) is disposed. Such unfocused ultrasound energy may, for example, ablate target nerves surrounding the body lumen, without damaging non-target tissue such as the inner lining of the body lumen or unintended organs outside of the body lumen. The unfocused ultrasound energy system may also include a balloon mounted at the distal end of the catheter around the ultrasound transducer. A cooling fluid can be circulated through the balloon to cool the body lumen during ultrasound energy delivery. Such a design enables creation of one or more ablation zones sufficient to achieve long-term nerve inactivation at different locations around the circumference of the blood vessel.

### SUMMARY

The present disclosure is defined in the independent claims. Further embodiments of the present disclosure are defined in the dependent claims.

A method is provided herein. The method includes delivering, by a fluid transfer unit of a tissue treatment system, inflation fluid at an inflation pressure to a balloon. The method includes injecting, by a contrast injector of the tissue treatment system, contrast medium at an injection pressure into a vessel containing the balloon. The method includes determining one or more of the inflation pressure or the injection pressure. The method includes stopping, based on one or more of the inflation pressure or the injection pressure, injection of the contrast medium.

A non-transitory computer-readable medium storing instructions is provided herein. The instructions, when executed by one or more processors of a tissue treatment system, cause the tissue treatment system to perform a method including delivering, by a fluid transfer unit of the tissue treatment system, inflation fluid at an inflation pressure to a balloon. The method includes injecting, by a contrast injector of the tissue treatment system, contrast medium at an injection pressure into a vessel containing the balloon. The method includes determining, by the one or more processors, one or more of the inflation pressure or the injection pressure. The method includes stopping, based on one or more of the inflation pressure or the injection pressure, injection of the contrast medium by the contrast injector.

A tissue treatment system is provided herein. The tissue treatment catheter includes a fluid transfer unit to deliver an inflation fluid at an inflation pressure to a balloon. The tissue treatment catheter includes a contrast injector to inject contrast medium at an injection pressure into a vessel containing the balloon. The tissue treatment catheter includes a memory storing instructions. The tissue treatment catheter includes one or more processors configured to execute the instructions to determine one or more of the inflation pressure or the injection pressure, and cause the contrast injector to stop injection of the contrast medium based on one or more of the inflation pressure or the injection pressure.

A fluid transfer cartridge is provided herein. The fluid transfer cartridge includes a cartridge shell defining a cartridge cavity. The cartridge shell includes one or more stabilizing prongs. The fluid transfer cartridge includes a syringe barrel disposed within the cartridge cavity. The fluid transfer cartridge includes a syringe piston disposed within the syringe barrel. The syringe piston includes a stopper and a shaft extending longitudinally from the stopper to a shaft end. The shaft includes several piston notches each of which receives at least one of the one or more stabilizing prongs.

A fluid transfer cartridge is provided herein. The fluid transfer cartridge includes a cartridge shell defining a cartridge cavity. The cartridge shell includes several stabilizing prongs separated by a gap. The fluid transfer cartridge includes a syringe barrel disposed within the cartridge cavity. The fluid transfer cartridge includes a syringe piston disposed within the syringe barrel. The syringe piston includes a stopper and a shaft extending longitudinally from the stopper through the gap to a shaft end.

A fluid transfer cartridge is provided herein. The fluid transfer cartridge includes a cartridge shell defining a cartridge cavity between a front shell portion and a rear shell portion. The rear shell portion includes a sidewall extending longitudinally from a rear base plate to a rear face. Several latch keepers extend through the sidewall. Several latch guides are formed along an edge between the sidewall and the rear face.

A fluid transfer cartridge is provided herein. The fluid transfer cartridge includes a cartridge shell defining a cartridge cavity between a front shell portion and a rear shell portion. The rear shell portion includes a sidewall extending longitudinally from a rear base plate to a rear face. Several latch holes extend through the sidewall. The fluid transfer cartridge includes a shell plate contained within the cartridge cavity between the front shell portion and the rear shell portion. The shell plate includes several lateral projections extending into the several latch holes such that several latch keepers are defined between the several lateral projections and the rear face.

A fluid transfer cartridge is provided herein. The fluid transfer cartridge includes a cartridge shell defining a cartridge cavity between a front shell portion and a rear shell portion. The rear shell portion includes a label recess in a rear face. The label recess has a depth of 0.01 to 0.02 inch.

A fluid transfer cartridge is provided herein. The fluid transfer cartridge includes a cartridge shell defining a cartridge cavity between a front shell portion and a rear shell portion. The rear shell portion includes a sidewall extending longitudinally from a rear base plate to a rear face. Several latch holes extend through the sidewall. The fluid transfer cartridge includes a shell plate contained within the cartridge cavity between the front shell portion and the rear shell portion. The shell plate includes several lateral projections extending into the several latch holes such that several latch keepers are defined between the several lateral projections and the rear face.

A fluid transfer cartridge is provided herein. The fluid transfer cartridge includes a cartridge shell defining a cartridge cavity between a front face and a rear face. The cartridge shell includes a handle extending from the front face over an opening. The handle includes a detachable front plate.

A fluid transfer cartridge is provided herein. The fluid transfer cartridge includes a cartridge shell defining a cartridge cavity between a front face and a rear face. The front face includes a tab extending into a conduit port defined by an edge. The fluid transfer cartridge includes a conduit routing plate engaging the front face along the edge. The tab is located in a conduit slot of the conduit routing plate.

The above summary does not include an exhaustive list of all aspects of the present disclosure. It is contemplated that the present disclosure includes all systems and methods that can be practiced from all suitable combinations of the various aspects summarized above, as well as those disclosed in the Detailed Description below and particularly pointed out in the claims filed with the application. Such combinations have particular advantages not specifically recited in the above summary.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings.

FIG. 1 is a perspective view of a portion of a tissue treatment system, in accordance with an embodiment.

FIG. 2 is a perspective view of a tissue treatment catheter delivered into a body lumen, in accordance with an embodiment.

FIG. 3 is a schematic view of a tissue treatment system including a contrast injector, in accordance with an embodiment.

FIG. 4 is a perspective view of a contrast injector of a tissue treatment system, in accordance with an embodiment.

FIG. 5 is a cross-sectional view of a contrast injector of a tissue treatment system, in accordance with an embodiment.

FIG. 6 is an exploded view of a cartridge shell of a fluid transfer cartridge, in accordance with an embodiment.

FIG. 7 is a perspective view of a contrast reservoir positioned in a fluid transfer cartridge, in accordance with an embodiment.

FIG. 8 is an exploded view of a cartridge manifold, in accordance with an embodiment.

FIG. 9A is a flowchart of a method of circulating contrast medium from a contrast reservoir to a guide catheter via a cartridge manifold, in accordance with an embodiment.

FIG. 9B is a flowchart of a method of circulating contrast medium from a contrast reservoir to a guide catheter via a cartridge manifold using a contrast injector located inside a tissue treatment system, in accordance with an embodiment.

FIG. 10 is a schematic view of a tissue treatment system, in accordance with an embodiment.

FIG. 11 is a flowchart of a method of deploying a tissue treatment system, in accordance with an embodiment.

FIGS. 12A-12B are flowcharts of a method of controlling injection of contrast medium by a tissue treatment system, in accordance with an embodiment.

FIG. 13 is a perspective view of a catheter of a treatment system, in accordance with an embodiment.

FIG. 14 is a front perspective view of a generator and a fluid transfer cartridge of a treatment system, in accordance with an embodiment.

FIG. 15 is a rear perspective view of a generator and a fluid transfer cartridge of a treatment system, in accordance with an embodiment.

FIG. 16 is a perspective view of a cartridge receiving portion of a generator and a fluid transfer cartridge of a treatment system, in accordance with an embodiment.

FIG. 17 is an exploded view of a cartridge shell of a fluid transfer cartridge, in accordance with an embodiment.

FIG. 18 is a sectional view of an end-lit syringe of a fluid transfer cartridge, in accordance with an embodiment.

FIG. 19 is a front perspective view of a fluid transfer cartridge, in accordance with an embodiment.

FIG. 20 is a rear perspective view of a fluid transfer cartridge, in accordance with an embodiment.

FIG. 21 is a front view of a conduit routing port of a fluid transfer cartridge, in accordance with an embodiment.

FIG. 22 is a front view of a conduit routing plate mounted in a conduit routing opening of a fluid transfer cartridge, in accordance with an embodiment.

FIG. 23 is a cross-sectional view of a fluid transfer cartridge mounted in a cartridge receptacle of a generator of a treatment system, in accordance with an embodiment.

FIG. 24 is a rear perspective view of a fluid transfer cartridge, in accordance with an embodiment.

FIG. 25 is a front perspective view of a cartridge receiving portion of a generator, in accordance with an embodiment.

FIG. 26 is an exploded view of a cartridge shell of a fluid transfer cartridge, in accordance with an embodiment.

FIGS. 27A-27B are perspective views of a cartridge manifold, in accordance with an embodiment.

FIG. 28 is an exploded view of a cartridge manifold, in accordance with an embodiment.

FIG. 29 is a front view of a fluid transfer plate of a cartridge manifold, in accordance with an embodiment.

FIG. 30 is a rear view of a fluid transfer plate of a cartridge manifold, in accordance with an embodiment.

FIG. 31 is a perspective view of a piston of a cartridge manifold, in accordance with an embodiment.

FIG. 32 is a perspective view of a piston of a cartridge manifold, in accordance with an embodiment.

FIG. 33 is a sectional view, taken about line A-A of FIG. 30, of a piston of a cartridge manifold in an open position, in accordance with an embodiment.

FIG. 34 is a sectional view, taken about line A-A of FIG. 30, of a piston of a cartridge manifold in an closed position, in accordance with an embodiment.

FIG. 35 is a side view of a generator for an ultrasound-based treatment system, in accordance with an embodiment.

FIG. 36 is a sectional view, taken about line B-B of FIG. 35, of a generator for an ultrasound-based treatment system, in accordance with an embodiment.

FIG. 37 is a perspective view of a non-invasive sensor, in accordance with an embodiment.

FIG. 38 is a perspective view of a non-invasive sensor, in accordance with an embodiment.

FIG. 39 is a front view of an internal portion of a fluid transfer cartridge, in accordance with an embodiment.

FIG. 40 is a sectional view of an internal portion of a fluid transfer cartridge having a pneumatically driven syringe, in accordance with an embodiment.

FIG. 41 is a sectional view of an internal portion of a fluid transfer cartridge having a non-contact position sensor, in accordance with an embodiment.

FIG. 42 is a perspective view of an ultrasound-based treatment system, in accordance with an embodiment.

FIG. 43 is a sectional view of a drive mechanism of a fluid transfer cartridge, in accordance with an embodiment.

FIG. 44 is a block diagram of a controller of a treatment system, in accordance with an embodiment.

FIG. 45 is a perspective view of a shaft end having an optical tab, in accordance with an embodiment.

FIG. 46 is a rear view of a cartridge shell of a fluid transfer cartridge, in accordance with an embodiment.

FIG. 47 is a rear perspective view of a cartridge shell of a fluid transfer cartridge, in accordance with an embodiment.

FIG. 48 is a perspective view of a syringe piston of a fluid transfer cartridge, in accordance with an embodiment.

FIG. 49 is a cross-sectional view of a syringe piston, in accordance with an embodiment.

FIG. 50 is a cross-sectional view of a fluid transfer cartridge, in accordance with an embodiment.

FIG. 51 is a side view of a fluid transfer cartridge, in accordance with an embodiment.

FIG. 52 is a perspective view of a latch guide, in accordance with an embodiment.

FIG. 53 is a perspective view of a latch guide, in accordance with an embodiment.

FIG. 54 is a perspective exploded view of a fluid transfer cartridge, in accordance with an embodiment.

FIG. 55 is a perspective view of a shell plate of a fluid transfer cartridge, in accordance with an embodiment.

FIG. 56 is a perspective view of a cartridge shell of a fluid transfer cartridge, in accordance with an embodiment.

FIG. 57 is a side view of a latch keeper, in accordance with an embodiment.

FIG. 58 is a perspective view of a cartridge shell, in accordance with an embodiment.

FIG. 59 is a rear view of a handle of a cartridge shell, in accordance with an embodiment.

FIG. 60 is a perspective exploded view of a fluid transfer cartridge, in accordance with an embodiment.

FIG. 61 is an end view of a handle of a fluid transfer cartridge, in accordance with an embodiment.

FIG. 62 is a front perspective view of a conduit routing plate, in accordance with an embodiment.

FIG. 63 is a rear perspective view of a conduit routing plate, in accordance with an embodiment.

FIG. 64 is a cross-sectional perspective view of a conduit routing plate, in accordance with an embodiment.

FIG. 65 is a perspective view of a piston of a cartridge manifold, in accordance with an embodiment.

FIG. 66 is a cross-sectional view of a piston of a cartridge manifold, in accordance with an embodiment.

FIG. 67 is a sectional view, taken about line A-A of FIG. 30, of a piston of a cartridge manifold in an open position, in accordance with an embodiment.

### DETAILED DESCRIPTION

Systems that use unfocused ultrasound energy to treat tissue, and methods of using the same are provided herein. In certain embodiments, acoustic-based tissue treatment transducers, apparatuses, systems, and portions thereof, are provided. The systems may be catheter-based. The systems may be delivered intraluminally (e.g., intravascularly) so as to place a transducer within a target anatomical region of the subject, for example, within a suitable body lumen such as a blood vessel. Once properly positioned within the target anatomical region, the transducer can be activated to deliver unfocused ultrasonic energy radially outward so as to suitably heat, and thus treat, tissue within the target anatomical region. The transducer or piezoelectric material can be activated at a frequency, duration, and energy level suitable for treating the ablation target, e.g., the targeted tissue. In one non-limiting example, unfocused ultrasonic energy generated by the transducer or piezoelectric material or radio frequency (RF) energy transmitted by the electrodes may target select nerve tissue of the subject, and may heat such tissue in such a manner as to neuromodulate (e.g., fully or partially ablate, necrose, or stimulate) the nerve tissue.

Neuromodulating renal nerves may be used to treat various conditions, e.g., hypertension, chronic kidney disease, atrial fibrillation, autonomic nervous system for use in treating a variety of medical conditions, arrhythmia, heart failure, end stage renal disease, myocardial infarction, anxiety, contrast nephropathy, diabetes, metabolic disorder, and insulin resistance, etc. It should be appreciated, however, that the balloon catheters suitably may be used to treat other nerves and conditions, e.g., sympathetic nerves of the hepatic plexus within a hepatic artery responsible for blood glucose levels important to treating diabetes, or any suitable tissue, e.g., heart tissue triggering an abnormal heart rhythm, and is not limited to use in treating (e.g., neuromodulating) renal nerve tissue. In another example, a tissue treatment catheter is used to ablate sympathetic nerves of the renal arteries and a hepatic artery to treat diabetes or other metabolic disorders. In certain embodiments, the tissue treatment catheters are used to treat an autoimmune and/or inflammatory condition, such as rheumatoid arthritis, sepsis, Crohn's disease, ulcerative colitis, and/or gastrointestinal motility disorders by neuromodulating sympathetic nerves within one or more of a splenic artery, celiac trunk, superior or inferior mesenteric artery. In certain embodiments, the tissue treatment catheter is used to ablate nerve fibers in the celiac ganglion and/or renal arteries to treat hypertension. In certain embodiments, the transducers are used to treat pain, such as pain associated with pancreatic cancer, by, e.g., neuromodulating nerves that innervate the pancreas. Ultrasound or RF energy may also be used to ablate nerves of both the pulmonary vein and the renal arteries to treat atrial fibrillation. In still other examples, ultrasound or RF energy may additionally or alternatively be used to ablate nerves innervating a carotid body in order to treat hypertension and/or chronic kidney disease.

Computed tomography angiography procedures require intravenous iodinated contrast agents to allow imaging of catheter placement. For example, existing methods of using tissue treatment systems to ablate nerves include manual confirmation that a balloon of the balloon catheter is apposed to the vessel wall and/or occludes the target vessel prior to delivering energy to ablate nerves. Confirmation requires injection of contrast medium into the target vessel to allow a determination of apposition/occlusion to be made under fluoroscopy. Contrast medium is administered manually through a contrast flow area between the guide catheter and the balloon catheter. The manual nature of contrast medium administration, however, can result in an excess volume of contrast medium being administered. More particularly, the volume of contrast medium may be uncontrolled, and the excess volume may be sufficient to cause deleterious conditions, such as contrast-induced nephropathy (CIN). CIN involves a decline in kidney function soon after administration of iodinated contrast agents. A likelihood of CIN incidence may be reduced by minimizing an amount of contrast agent administered during a procedure.

Furthermore, depending on the guide catheter and balloon catheter designs, manual delivery of the contrast medium can be quite difficult to achieve manually. For example, increasing the length of the guide catheter for radial access may increase the force needed to push contrast medium through the contrast flow area. Also, a contrast flow area between the guide catheter and the balloon catheter may be quite small, and the relationship of the catheter diameters may resist flow of the dense contrast medium. With both examples, a clinician may be required to exert substantial strength to force the contrast medium through the contrast flow area, which can result in user exhaustion and/or user error. Lack of control by the clinician can also result in contrast medium being delivered at a flow rate that overcomes the balloon inflation and results in balloon collapse and loss of contrast medium downstream within the target vessel. Therefore, there is a need for a method of controlling contrast medium injection by a tissue treatment system to enhance patient safety and ease of use.

The balloon catheter in the present disclosure may be incompatible with current contrast auto-injectors because current contrast auto-injectors have a large pressure influx on the balloon of the balloon catheter. This large pressure influx will cause the balloon to deflate during use. Balloon deflation during use does not allow the user to confirm apposition/occlusion under fluoroscopy.

The present disclosure may also allow design freedom of future guide catheters and balloon catheters because the space once needed for the contrast flow area is minimized. Minimizing the contrast flow area may allow the outer diameter of the guide catheter and of the balloon catheter of future designs to be smaller.

Existing hypertension treatment systems include generators to generate and deliver energy, e.g., RF or ultrasound energy, to a catheter-based intraluminal device. The treatment systems may also include components that engage with the generators to facilitate treatment. For example, cartridges may mount on the generators to deliver inflation or cooling fluid to a balloon mounted on an end of a catheter. The generator and/or cartridge may be large and bulky, especially in combination. Furthermore, mechanical and electrical connections between the generator and cartridge can be unreliable due to imperfect mounting, tolerance stack ups, or movement that occurs between the components during operation. In the case of cartridges that deliver fluids, the fluid transfer may not be accurately monitored either visually or automatically due to a lack of lighting in the procedure room and/or unstable sensor connections between the generator and the cartridge. The system may also integrate long lengths of internal tubing that increases an overall form factor of the equipment. Accordingly, treatment systems used to deliver energy and fluid to a catheter-based intraluminal device would benefit from more compact, mechanically stable, electrically stable, and ergonomic designs.

As described below, embodiments can include a treatment system having a generator and a fluid transfer cartridge, and methods of using the treatment system. The treatment system may be an ultrasound-based tissue treatment system, used to delivery unfocused ultrasonic energy radially outwardly to treat tissue within a target anatomical region, such as the renal nerves within a renal artery. Alternatively, the tissue treatment system may be used in other applications, such as to treat sympathetic nerves of the hepatic plexus within a hepatic artery. Thus, reference to the system as being a renal denervation system, or being used in treating, e.g., neuromodulating, renal nerve tissue is not limiting.

In various embodiments, description is made with reference to the figures. However, certain embodiments may be practiced without one or more of these specific details, or in combination with other known methods and configurations. In the following description, numerous specific details are set forth, such as specific configurations, dimensions, and processes, in order to provide a thorough understanding of the embodiments. In other instances, well-known processes and manufacturing techniques have not been described in particular detail in order to not unnecessarily obscure the description. Reference throughout this specification to “one embodiment,” “an embodiment,” or the like, means that a particular feature, structure, configuration, or characteristic described is included in at least one embodiment. Thus, the appearance of the phrase “one embodiment,” “an embodiment,” or the like, in various places throughout this specification are not necessarily referring to the same embodiment. Furthermore, the particular features, structures, configurations, or characteristics may be combined in any suitable manner in one or more embodiments.

The use of relative terms throughout the description may denote a relative position or direction. For example, “above” may indicate a first direction relative to a component. Similarly, “below” may indicate a second direction relative to the component, opposite to the first direction. Such terms are provided to establish relative frames of reference, however, and are not intended to limit the use or orientation of treatment system components, e.g., a fluid transfer cartridge or a generator, to a specific configuration described in the various embodiments below.

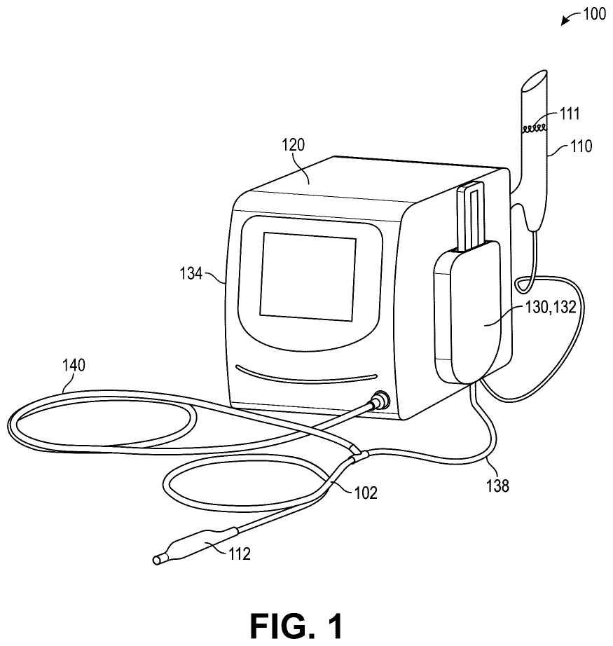

In an aspect, (see, e.g., FIGS. 1-12B) a tissue treatment system is configured to perform a method of controlling injection of a contrast medium. The tissue treatment system can include a contrast injector to semi-automatically deliver contrast medium to a target vessel. The contrast injector can be integrated into an energy delivery unit, or another component, of the system. The contrast injector may also be physically separated from the energy delivery unit. The contrast injector can be placed inside or outside of a sterile area in an operating arena. The contrast reservoir can be located inside the fluid transfer cartridge and the contrast injector can be located outside the fluid transfer cartridge. The contrast injector delivers the contrast medium when a balloon of the system is inflated to allow a clinician to confirm apposition/occlusion under fluoroscopy. Semi-automated delivery of the contrast medium can be controlled based on monitored pressures and/or flow rates. For example, the contrast medium injection may be stopped when a maximum volume of contrast medium is delivered, or when a monitored pressure of the contrast medium or the balloon indicates that the contrast medium is collapsing the balloon. Accordingly, the integrated contrast injector can make delivery of contrast medium easier and faster for the clinician, can be used with catheters that have closely matched diameters, and can be controlled to reduce exposure of contrast medium to a patient.

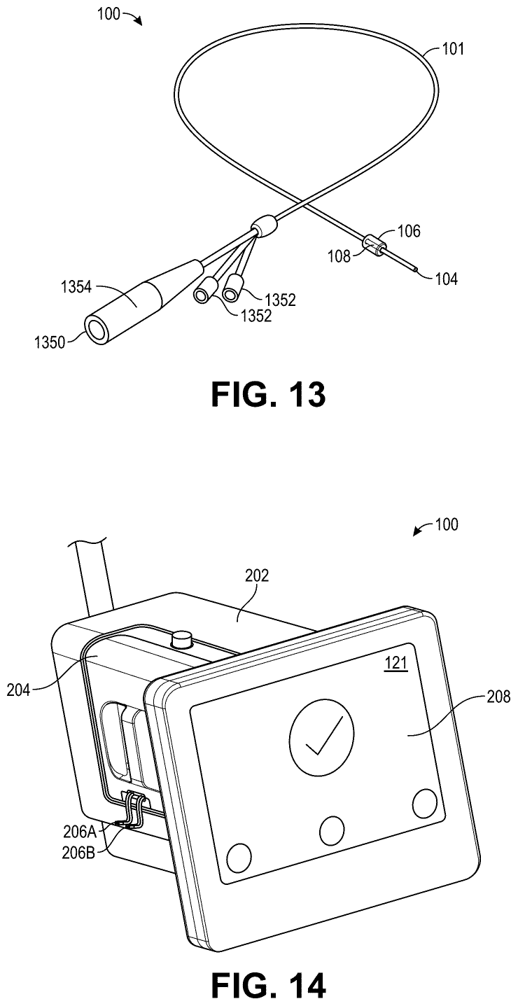

In an aspect, (see, e.g., FIGS. 13-64) a treatment system for performing a medical procedure, e.g., a renal ablation catheterization, is provided. The treatment system includes a fluid transfer cartridge to deliver fluid to a catheter, and a generator to deliver energy to the catheter. The fluid transfer cartridge and the generator combine to form a control unit of the treatment system. The control unit is compact. More particularly, the fluid transfer cartridge fits within a cartridge receptacle of the generator to form a clean and compact profile of the control unit. Furthermore, the fluid transfer cartridge has syringe components that can be fully contained within a cartridge housing to reduce an overall form factor of the control unit. The control unit is mechanically stable. The fluid transfer cartridge can be fastened to the generator by a fastening mechanism that evenly distributes a retention force around the cartridge housing, and which has a quick release mechanism to make engagement and disengagement of the components fast and reliable. The control unit is electrically stable. Electrical connections between the fluid transfer cartridge and the generator may be via spring-loaded electrical contact pins, commonly referred to as pogo pins. The spring-loaded pins can maintain pressure at the electrical contact points between the components such that the connections are resilient against relative movement that can occur during operation. Furthermore, sensors used to detect movement of system components, such as a syringe piston of the fluid transfer cartridge, may include position sensors, such as magnetic switches or optical sensors, which are more stable and less susceptible to misalignment than, for example, mechanical switches. The control unit is user-friendly. The control unit can include one or more processors and various sensors that operate to determine a system readiness state, e.g., whether various electrical or component connections have been made, and to provide feedback to a user. By way of example, the system can detect whether the fluid transfer cartridge is mounted in the cartridge receptacle of the generator, and activate lights within the fluid transfer cartridge to illuminate the syringes to provide feedback about that state to the user. Accordingly, a treatment system having a compact, mechanically stable, electrically stable, and ergonomic design is provided.

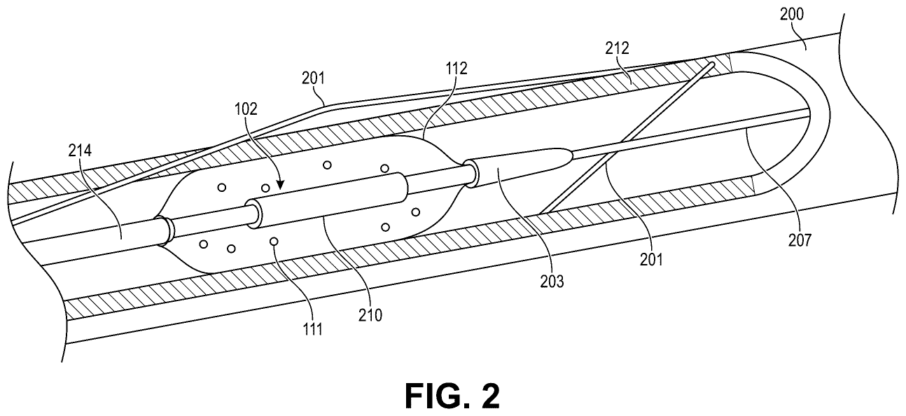

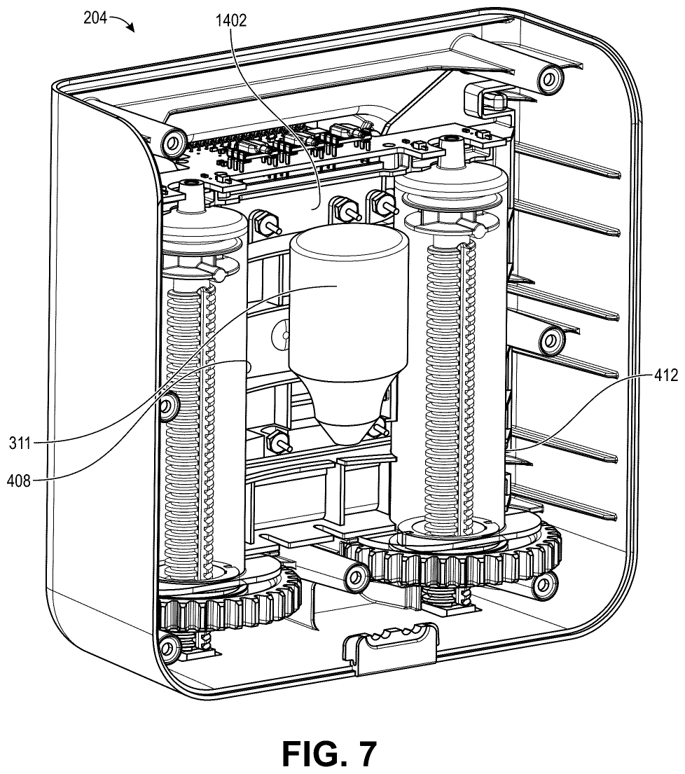

Referring to FIG. 1, a tissue treatment system is illustrated in accordance with an embodiment. A tissue treatment system 100 is shown as including a tissue treatment catheter 102 connected to a controller 120 by a connection cable 140. In certain embodiments, the tissue treatment catheter 102 includes an ultrasound transducer 210 (FIG. 2) within a balloon 112. The tissue treatment system 100 can include a fluid reservoir 110 to store an inflation fluid 111. The inflation fluid 111 may be a cooling fluid. Optionally, the tissue treatment system 100 can include a contrast reservoir, which may house contrast medium (for example, a contrast reservoir 311 illustrated in FIG. 7). The tissue treatment system 100 can include, e.g., integrated within the controller 120, a fluid transfer unit 130 to transfer or move the inflation fluid 111 and/or contrast medium into and out of the balloon 112. More particularly, the fluid transfer unit 130 of the tissue treatment system 100 may deliver the inflation fluid 111 at an inflation pressure to the balloon 112, as described below. The tissue treatment system 100 may also include a cooling unit 132, e.g., integrated within the controller 120, to cool the inflation fluid 111. Accordingly, the inflation fluid 111 can be delivered to the balloon 112 by the fluid transfer unit 130 at a temperature below ambient temperature. In an embodiment, the tissue treatment system 100 includes an energy delivery unit 134 configured to control activation, e.g., energize, the ultrasound transducer to deliver energy to the target anatomy.

In the embodiment shown in FIG. 1, the controller 120 is connected to the tissue treatment catheter 102 through an inflation tubing 138 for fluid transfer, and the connection cable 140 for electrical communication. In certain embodiments, the controller 120 interfaces with the fluid transfer unit 130 to provide the inflation fluid 111 to the tissue treatment catheter 102 for selectively inflating and deflating the balloon 112. The balloon 112 can be made from, e.g., nylon, a polyimide film, a thermoplastic elastomer (such as those marked under the trademark PEBAX™), a medical-grade thermoplastic polyurethane elastomer (such as Pellethane®, Isothane®, or other suitable polymers or any combination thereof), but is not limited thereto.

Referring to FIG. 2, a perspective view of a tissue treatment catheter inserted into a body lumen, is illustrated in accordance with an embodiment. A distal portion of the tissue treatment catheter 102 may be inserted into a body lumen of a subject. The body lumen may be a vessel 200, e.g., a blood vessel such as a renal artery, which has several nerves 201. The vessel 200 can be a target vessel of an ablation procedure. More particularly, the nerves 201 can be an ablation target. The nerves 201 can surround the body lumen. For example, the nerves 201 may run in and around the blood vessel 200, such as through an outer layer, e.g., adventitia layer, of the vessel 200.

The distal portion of the tissue treatment catheter 102 may include an ultrasound transducer 210, the balloon 112 filled with the inflation fluid 111, a catheter shaft 214, and/or a guidewire support tip 203 configured to receive a guidewire 207. The transducer 210 may be disposed partially or completely within the balloon 112, which may be inflated with the inflation fluid 111. The inflation fluid 111 can include a liquid. The liquid may have a relatively high, as compared to gases, thermal capacity. For example, the liquid may include water, dextrose, or saline, and have a corresponding heat capacity. When the inflation fluid 111 is transferred into an interior of the balloon 112, the balloon can inflate into contact with a vessel wall 212 of the blood vessel 200. The vessel wall 212, and/or the nerves 201 extending within and around the vessel wall, can be an ablation target. In certain embodiments, the transducer 210 may be used to output acoustic energy to ablate the ablation target. Accordingly, the inflation fluid 111 can act as a heat sink to absorb heat generated by the ultrasound transducer 210 and/or delivered to the ablation target from the ultrasound transducer 210.

In certain embodiments, e.g., suitable for renal denervation, the balloon 112 is inflated while inserted in the body lumen of the patient during a procedure at a working pressure of about 10 to about 30 psi using the inflation fluid 111. The balloon 112 may be or include a compliant, semi-compliant, or non-compliant medical balloon. The balloon 112 is sized for insertion in the body lumen and, in the case of insertion into the renal artery, for example, the balloon 112 may be selected from available sizes including outer diameters of 3.5, 4.2, 5, 6, 7, or 8 mm, but not limited thereto. When activated, the transducer 210 can deliver the acoustic energy to the vessel wall 212 of the target vessel 200. The delivered energy can ablate and raise a temperature of the ablation target. The cooling fluid within the balloon 112, however, can be static and absorb heat to passively cool the ablation target and protect the target tissue and the transducer 210, as described below.

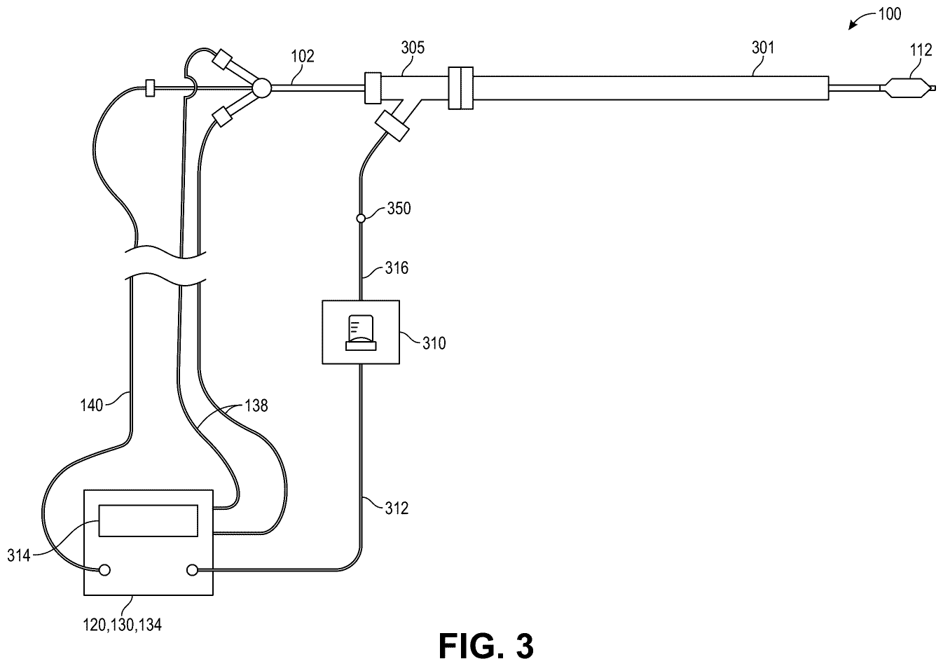

Referring to FIG. 3, a schematic view of a tissue treatment system including a contrast injector is shown in accordance with an embodiment. The portion of the tissue treatment system 100 described above with respect to FIG. 1 can be integrated with additional components of the overall system. For example, the tissue treatment catheter 102 may extend through a guide catheter 301. In an ablation procedure, the guide catheter 301 may be inserted into the vessel 200, and the tissue treatment catheter 102 may then be tracked through the guide catheter 301 such that the balloon 112 is located distal to the guide catheter and adjacent to the ablation target.

The guide catheter 301 and/or the tissue treatment catheter 102 may also communicate with a coupler 305. The coupler 305 can be an intermediate component, such as a rotating hemostasis valve. The coupler 305 may connect to the guide catheter 301 at a distal connector, and can seal against the tissue treatment catheter 102 at a proximal connector. An auxiliary, or side port of the coupler 305 can receive a contrast medium to convey the contrast medium through the contrast flow area between the guide catheter 301 and tissue treatment catheter 102 into the vessel 200.

As described above, the tissue treatment system 100 can include the controller 120 to convey inflation fluid 111 and/or electrical signals to the tissue treatment catheter 102. For example, the connection cable 140 can connect the energy delivery unit 134 of the controller 120 to an electrical connector of the tissue treatment catheter 102. The energy delivery unit 134 may supply energy through the connection cable 140 to energize the ultrasound transducer 210 contained within the balloon 112. Accordingly, the ultrasound transducer 210 can emit acoustic energy through the balloon 112 to the ablation target.

The acoustic energy emitted by the transducer 210 can also transmit through the inflation fluid 111 to the ablation target. More particularly, the fluid transfer unit 130 of the tissue treatment system 100 can deliver the inflation fluid 111 through inflation tubing 138 to one or more fluid connectors of the tissue treatment catheter 102. The inflation fluid 111 may therefore be conveyed to and from the balloon 112 during the ablation procedure. The inflation fluid 111 can cool the transducer 210 as the transducer emits the acoustic energy through the inflation fluid 111 to the ablation target.

When the balloon 112 is inflated adjacent to the ablation target, contrast medium can be conveyed through the contrast flow area between the guide catheter 301 and tissue treatment catheter 102 into the vessel 200 to visualize the balloon inflation under fluoroscopy. More particularly, fluoroscopy can be used to determine whether the balloon 112 is in apposition to the vessel wall 212 and/or occludes the vessel 200. The contrast medium can be delivered into the guide catheter 301 through the auxiliary port of the coupler 305.

An injection force required to deliver the contrast medium corresponds to a contrast flow area existing between an outer surface of the tissue treatment catheter 102 and an inner surface of the guide catheter 301. More particularly, as the contrast flow area decreases, the force required to push the contrast medium through the contrast flow area increases. Reducing catheter profiles can be important to accessing smaller anatomies. In an embodiment, the outer diameter of the guide catheter 301 is 6 French, and the outer diameter of the tissue treatment catheter 102 is 5 French. The intervening contrast flow area in such case may resist contrast flow. Accordingly, the tissue treatment system 100 may include a contrast injector 310 to inject contrast medium into the guide catheter 301 and through the contrast flow area. More particularly, contrast injector 310 can inject contrast medium at an injection pressure into the vessel 200 containing the balloon 112. An injection force required to deliver the contrast medium can correspond to a length of the guide catheter 301 and a length of the tissue treatment catheter 102. More particularly, as the length of the guide catheter 301 and the tissue treatment catheter 102 increases, the force required to push the contrast medium through the contrast flow area increases. Increasing the length of the guide catheter 301 and the tissue treatment catheter 102 can be important for radial access. In an embodiment, the length of the guide catheter 301 can be about 155 centimeters, and the length of the tissue treatment catheter 102 can be about 165 centimeters, to permit delivery through the radial artery while still providing sufficient force to push the contrast medium through the contrast flow area.

The contrast injector 310 can be controlled by the controller 120. For example, an injection control line 312 can electrically connect the contrast injector 310 to the controller 120. Commands can be entered by a clinician, e.g., through a graphical user interface presented on a display 314 of the controller 120, to cause the contrast injector 310 to start or stop injection of the contrast medium. Based on the commands, and/or one or more instructions stored in a memory of the controller and executed by one or more processors of the controller, the contrast injector 310 can semi-automatically or automatically deliver the contrast medium through an injection tubing 316. The injection tubing 316 can connect to the auxiliary port of the coupler 305, and thus, the contrast medium can be delivered distally through the guide catheter 301 into the vessel 200.

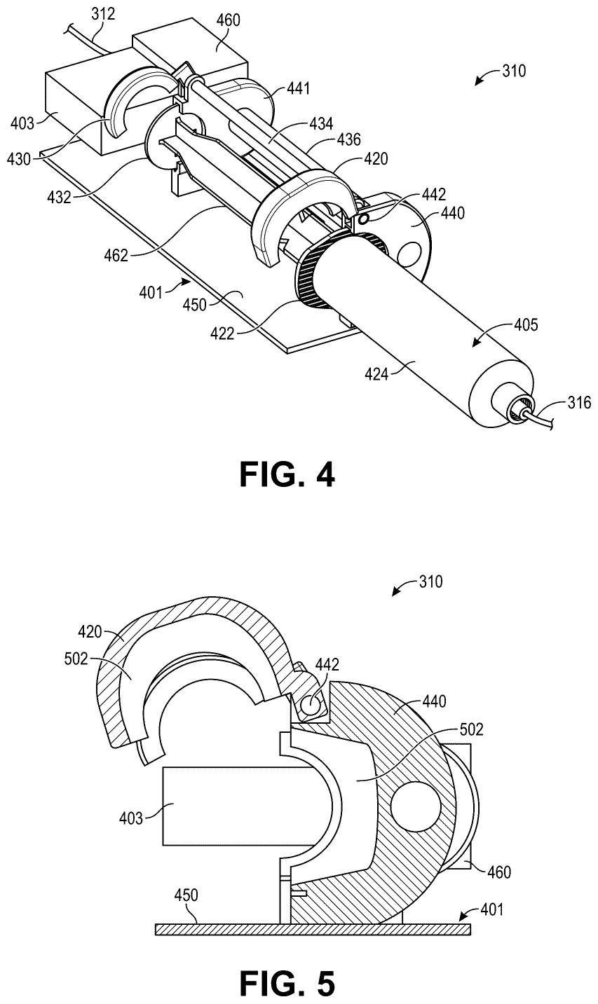

Referring to FIG. 4, a perspective view of a contrast injector of a tissue treatment system is shown in accordance with an embodiment. The contrast injector 310 can include a housing 401 or frame to house a syringe 405. The housing 401 can receive the syringe 405, which may contain contrast medium. For example, the clinician can fill the syringe 405 with the contrast medium prior to loading the syringe 405 into the contrast injector 310. The injection tubing 316 may be connected to the syringe 405 to convey the contrast medium from the syringe 405 to the coupler 305.

In an embodiment, the injection control line 312 can interconnect the controller 120 to the contrast injector 310. For example, the injection control line can receive power and control signals from the controller 120. The signals can power and control circuitry of the contrast injector 310. More particularly, the contrast injector 310 can include circuitry within an electronics housing 403, and the circuitry can control the driving mechanism to expel contrast medium from the syringe 405. The driving mechanism can include a drive motor 460, such as a stepper motor. When the stepper motor is actuated, the drive motor 460 can move a driveshaft to advance a plunger 462 of the syringe 405 and administer contrast medium to a patient.

At least one of the drive heads, e.g., drive head 440, may be mounted on and/or fixed to a base plate 450 that grounds the housing 401. More particularly, the housing 401 can include the base plate 450 to mount one or more of the other housing components on. The housing components can be assembled on top of the base plate 450 as shown in FIGS. 4-5.

In an embodiment, the contrast injector 310 includes a syringe barrel cap 420 to receive a barrel flange 422 of a syringe body (barrel) 424. Similarly, the contrast injector 310 can include a syringe plunger cap 430 to receive a plunger flange 432 of the syringe 405. The syringe caps can move relative to each other. For example, whereas one of the drive heads, e.g., drive head 440, can be fixed to the base plate 450, another of the drive heads, e.g., drive head 441, can move relative to the fixed drive head. The syringe caps 420, 430, which are mounted on the drive heads 440, 441, respectively, may therefore move relative to each other. The drive heads 440, 441 and/or syringe caps 420, 430 can be interconnected by a guide rail 434, and thus, the guide rail 434 can guide movement of the syringe caps relative to each other. The guide rail 434 can maintain a longitudinal alignment of the caps, and allow the syringe plunger cap 430 to move distally/proximally along the guide rail 434 relative to the syringe barrel cap 420. The guide rail 434 can also maintain concentricity and eliminate rotation between the drive heads 440, 441. The movement may be urged by a drive shaft 436 connected between the syringe plunger cap 430 and the drive motor 460. Accordingly, the syringe plunger cap 430 can be driven forward relative to the syringe barrel cap 420 to move the plunger 462 within the syringe body 424 and expel the contrast medium into the injection tubing 316.

The caps of the housing 401 can be pivotable. As shown in FIG. 4, the syringe barrel cap 420 and the syringe plunger cap 430 may be connected to respective drive heads 440, 441 of the frame at respective hinges 442. The pivotable caps can swing to the open position (as shown) to allow the clinician to insert the filled syringe 405 into the housing 401. More particularly, the syringe barrel cap 420 can be inserted into a receiving slot of the distal drive head 440 and the syringe plunger cap 430 can be inserted into a receiving slot of the proximal drive head 441.

Referring to FIG. 5, a cross-sectional view of a contrast injector of a tissue treatment system is shown in accordance with an embodiment. The housing 401 includes slots 502, e.g., the slot within the distal drive head 440 that is mounted on the base plate 450, sized to receive the respective flange. For example, the slot 502 in the distal drive head 440 and the pivotable syringe barrel cap 420 can be sized to receive the syringe barrel flange 422, and the slot 502 in the proximal drive head 441 and the pivotable syringe plunger cap 430 (hidden) can be sized to receive the syringe plunger flange 432. When the pivotable caps are in the open position, the respective flange can be fit into a portion of the slot 502, e.g., into the portion of the slot in the distal drive head 440. The pivotable caps may then be swung to a closed position to encompass and secure the respective flange. For example, the syringe barrel cap 420 can be pivoted downward to receive the barrel flange 422 in the slot 502 portion of the cap. The pivotable cap may be secured, e.g., latched, in the closed position to retain the flange within the slot 502. The slot 502 can extend laterally into the drive head 440 and the cap 420, and may be defined by proximal and distal walls of the cap 420 and the drive head 440. When the barrel flange 422 is within the slot 502 in the closed position, movement of the drive head 441 can advance or retract the flange. Accordingly, when the drive motor 460 drives the proximal drive head 441 distally relative to the distal drive head 440, the syringe plunger cap 430 received within the slot 502 of the proximal drive head 441 can advance relative to the syringe barrel cap 420 received within the slot 502 of the distal drive head 440. Contrast medium can therefore be expelled from the syringe 405.

The slots 502 of the contrast injector 310 may be sized to receive and hold syringes 405 of different sizes. Clinicians may choose syringes of varying capacity. For example, various syringe sizes in a range of 10-30 mL of contrast medium may be stocked and used by various users. Several common syringe barrel sizes include syringes having internal volumes of 10 mL, 20 mL, or 30 mL. Accordingly, the syringe barrel size, and the corresponding barrel or plunger flange sizes may vary. To accommodate a variety of sizes, the slot 502 may be large enough to allow the largest of such barrel or plunger flanges to fit within the slot 502, and small enough to ensure that the proximal and distal walls of the respective drive head will contact the flange. Accordingly, the slot 502 can be sized to receive a first flange of a first syringe, e.g., a 10 mL syringe, and a second flange of a second syringe, e.g., a 30 mL syringe. The second flange can be sized differently than the first flange, but may nonetheless fit within and be secured by the slot 502.

The actuation of the drive shaft by the drive motor 460 to expel contrast medium at a given rate or pressure may be controlled by the circuitry in the electronic housing 401. More particularly, the circuitry may be electrically connected to, and under the control of, one or more processors of the controller 120. Methods of control are described further below and, in some embodiments, rely on an injection pressure of the contrast medium. Referring again to FIG. 3, an injection sensor 350, e.g., a pressure sensor or a flow sensor, may be integrated in the tissue treatment system 100 to monitor injection parameters, such as the injection pressure. The injection sensor 350 can be located along the fluid path between the contrast injector 310 and the balloon 112. For example, the injection sensor 350 can be in line with the injection tubing 316, may be integrated within the coupler 305 or the guide catheter 301, or may be placed in the space between a distal end of the guide catheter 301 and the balloon 112. Sensed data from the injection sensor 350 can be used by the one or more processors 602 to control contrast medium injection, as described below.

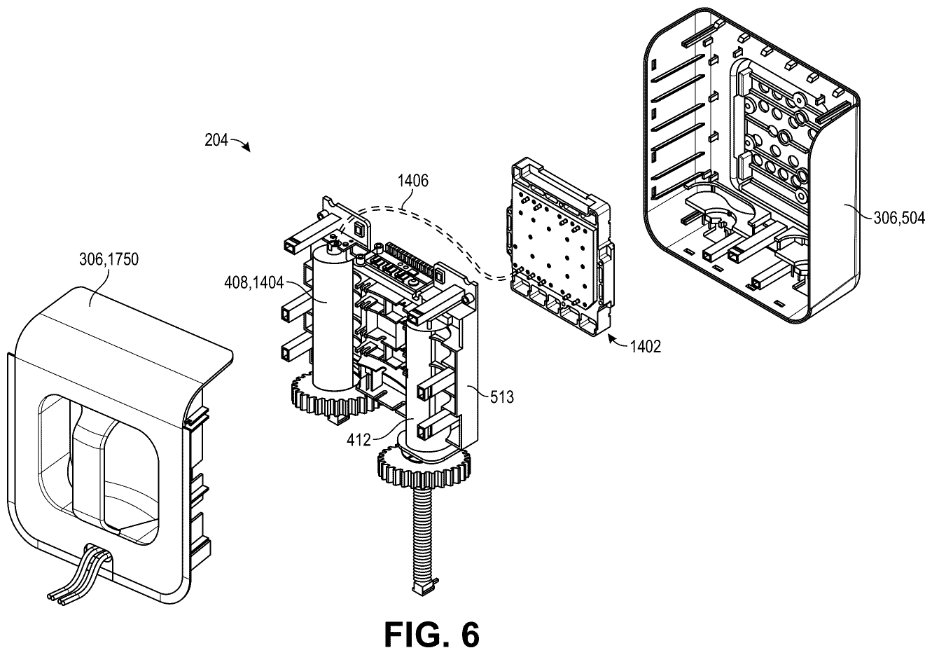

FIG. 6 illustrates an exploded view of a cartridge shell 306 of a fluid transfer cartridge 204 according to an embodiment. The cartridge shell 306 of the fluid transfer cartridge 204 can include the handle front plate 1750 and the back plate 504. When combined, the handle front plate 1750 and the back plate 504 can define the cartridge cavity 402 centrally located between the various walls and faces. When snapped or otherwise fit together, the handle front plate 1750 and the back plate 504 can contain, within the cartridge cavity 402, one or more components to provide fluid transfer functionality. For example, the fluid transfer cartridge 204 can include the syringe holder 513 to hold the syringe barrels 408, 412 and the cartridge manifold 1402. The syringe holder 513 can stabilize the syringes during fluid delivery. The fluid transfer cartridge 204 can also include tubing 1406 to facilitate the movement of fluid from the syringes to the catheter 101. As illustrated in FIG. 7, in some embodiments, the contrast reservoir 311 can be positioned in the fluid transfer cartridge 204 between the two syringe barrels 408, 412 and the contrast injector 310 can be located outside the fluid transfer cartridge 204. The contrast reservoir 311 holds the contrast medium and can be connected to the contrast injector 310 via tubing.

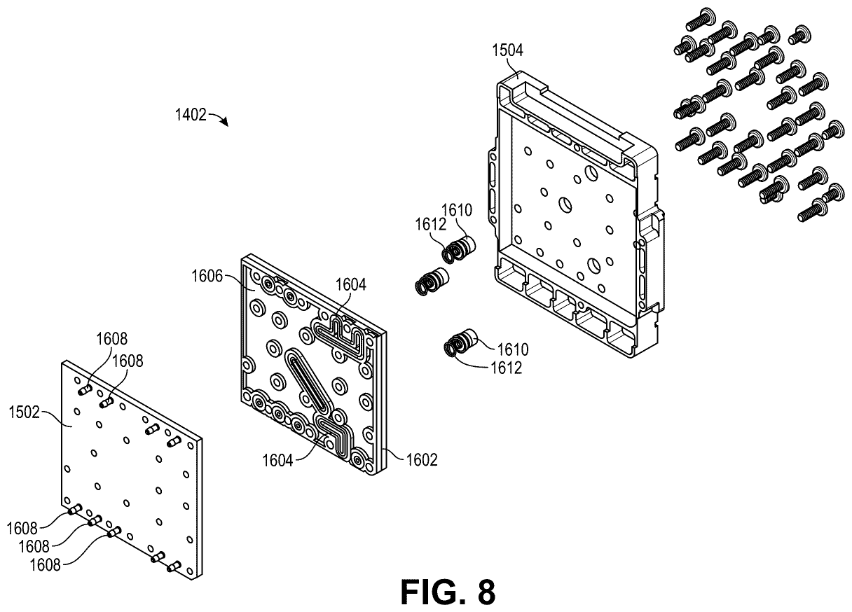

In an embodiment as shown in FIG. 8, the cartridge manifold 1402 in the cartridge cavity 402 can include a fluid transfer plate 1602 sandwiched between the fore plate 1502 and the aft plate 1504. The fluid transfer plate 1602 can include channels on front and rear surfaces that are connected through various ports in the plate. More particularly, one or more front fluid channel 1604 in a front plate surface 1606 can carry fluid, for example, contrast medium, via channels on the rear surface, to one or more outlet ports 1608 for transfer to the external components. In some embodiments, the outlet ports 1608 of the fore plate 1502 connect to external components. More particularly, the outlet ports 1608 can include fittings, e.g., barb fittings, which connect to fluid conduits, and those conduits can extend to connect to external components, such as syringes, fluid reservoirs, the balloon catheter 101, contrast injector 310, or pressure sensors. Accordingly, the fore plate outlet ports 1608 can function as fluid interfaces to the external components. Through the outlet ports 1608, fluid, such as contrast medium, can be transferred into and out of the cartridge manifold 1402. In an embodiment, the fore plate 1502 includes four outlet ports 1608 along an upper edge 1004 and five outlet ports 1608 along a lower edge 1004, although the number and locations of these outlet ports 1608 can be varied according to a layout of the external components and the fluid transfer cartridge 204.

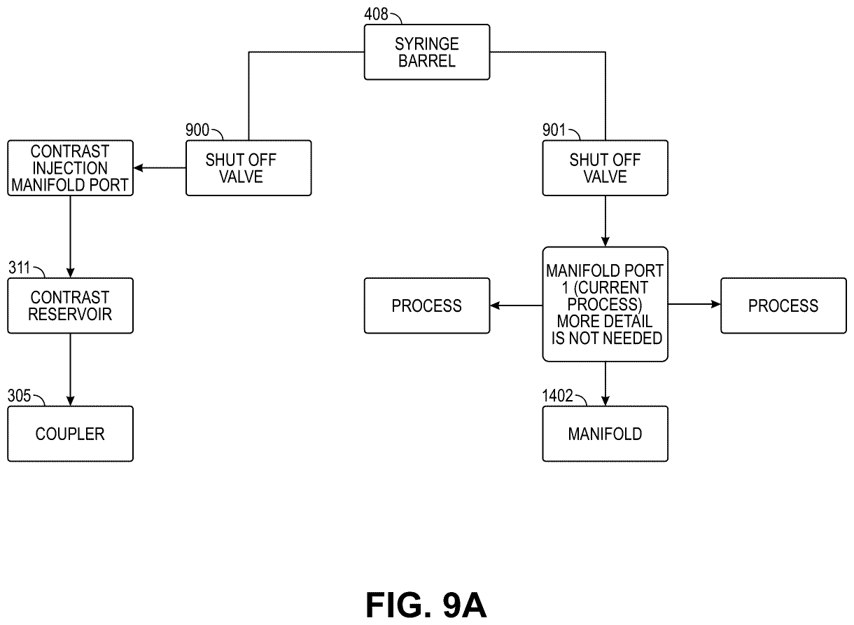

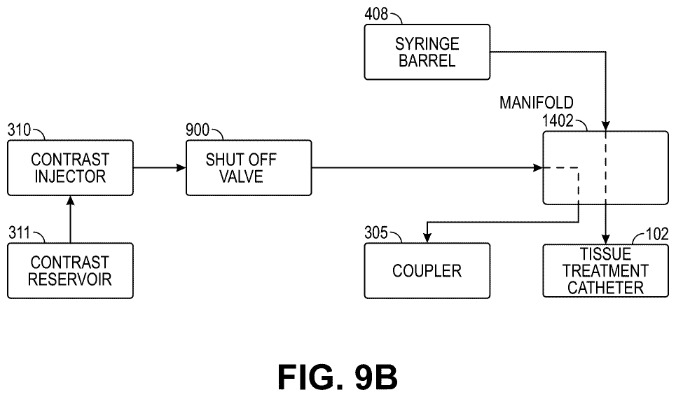

FIG. 9A illustrates a flowchart of a method of circulating contrast medium from the contrast reservoir 311 to the guide catheter 301. The contrast medium may be delivered directly from the contrast reservoir 311, or indirectly via the cartridge manifold 1402. In an embodiment, the driving mechanism may include a pressurized system. The pressurized system can utilize hydraulics to move contrast from the contrast reservoir 311 to the coupler 305 and into the guide catheter 301. For example, the syringe barrel 408 can expel inflation fluid 111, e.g. cooling fluid, through the shut off valve 900 and through the contrast injection manifold port to enter the contrast reservoir 311. The cooling fluid can be used to mix with and/or force contrast medium from the contrast reservoir 311 into tubing connected to the coupler 305. Alternatively, a pneumatics source can, via pneumatic valve manipulation, drive the contrast delivery. For example, the contrast reservoir 311 may include a plunger that can be actuated by the pneumatic source (or alternatively by the hydraulic source of the syringe barrel 408) to drive the contrast medium into the tubing connected to the coupler 305. The system components can therefore expel contrast medium through the injection tubing 316 to the coupler 305, e.g., a hemostasis valve, and can control the flow of the contrast medium through the guide catheter 301 into the patient.

Fluid from the syringe barrel 408 can be delivered to the contrast reservoir 311 or can be delivered to the cartridge manifold 1402. Fluid may be delivered simultaneously or asynchronously under the control of one or more shut off valves 900. More particularly, a first shut off valve 900 can control flow between the syringe barrel 408 and the contrast reservoir 311, and a second shut off valve 901 can control flow between the syringe barrel 408 and the manifold 1402. Opening the shut off valves 900, 901 when the syringe barrel 408 is actuated allows cooling fluid to flow through the shut off valves 900, 901 to their respective terminal components. Alternatively, closing a shut off valve 900, 901 will stop flow to the respective terminal component, e.g., the contrast reservoir 311 or the manifold 1402.

Should the clinician inject contrast, the syringe barrel 408 can be actuated and the shut off valve 900 can be opened to push the fluid from the syringe barrel 408 into the contrast reservoir 311. The fluid force from the syringe barrel 408 can be used to deliver the contrast medium from the contrast reservoir 311 through tubing to the coupler 305. Notably, the contrast medium may flow into the tubing directly from the contrast reservoir 311, or may be routed through the manifold 1402 to an exit port of the fluid cartridge into the tubing. The shut off valves 900, 901 control the movement of fluid and prevents contrast to be pulled back into the syringe barrel 408. When contrast medium needs to be delivered, the shut off valve 900 is open and the fluid from the syringe barrel 408 circulates to the contrast reservoir 311. After fluid enters the contrast reservoir 311, the fluid mixes with the concentrated contrast medium to create a more diluted solution of the contrast medium. After mixing, the contrast medium can be delivered into the tubing, e.g., directly or through the cartridge manifold 1402. After flowing through the tubing, the contrast medium enters the coupler 305, for example, a hemostasis valve, which can connect to the guide catheter 301. Cooling fluid can be delivered to the tissue treatment catheter 102 by the manifold 1402 in a cooling fluid delivery process, independently of contrast medium delivery to the guide catheter 301.

FIG. 9B is a flowchart of a method of circulating contrast medium using a contrast injector 310 located inside the tissue treatment system 100. For example, the contrast medium can exit the contrast reservoir and flow into the contrast injector 310. The contrast medium can flow through the shut off valve 900 and into the manifold 1402 and into the coupler 305, e.g., hemostasis valve. The cooling fluid from the syringe barrel 408 can be delivered through the cartridge manifold 1402 and to the tissue treatment catheter 102.

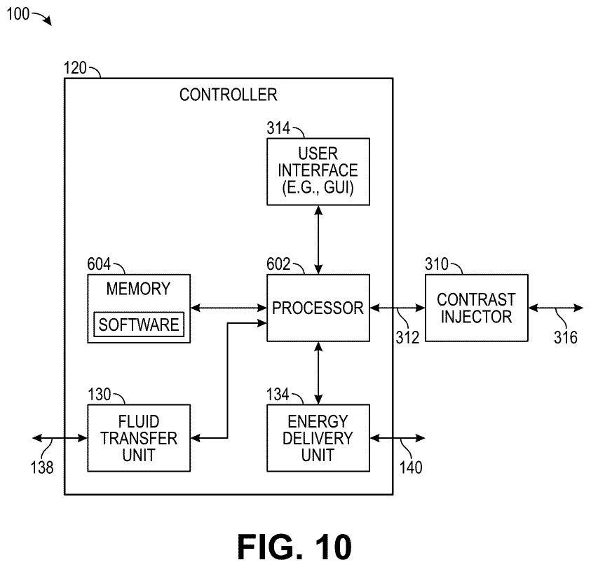

Referring to FIG. 10, a schematic view of a tissue treatment system is shown in accordance with an embodiment. The controller 120 can include one or more processors 602, e.g., located on a control board or, more generally, a printed circuit board (PCB) along with additional circuitry. The processor(s) 602 can communicate with memory 604 of the controller 120, which can be a non-transitory computer-readable medium storing instructions. The processor 602 can execute the instructions to cause the tissue treatment system 100 to perform the methods described herein. A user interface, e.g., the display 314, interacts with the processor 602 to receive user inputs, such as user selections of displayed graphical user elements, and to output or display information to the user. The interactions can cause the one or more processors 602 to control balloon inflation, transducer sonication, and contrast injection, as described below. More particularly, the processor 602 can control the fluid transfer unit 130 to convey inflation fluid 111 through the inflation tubing 138, can control the energy delivery unit 134 to energize the transducer 210 via the connection cable 140, and can control the contrast injector 310 to inject contrast medium through the injection tubing 316. The system can detect whether the contrast injector 310 is communicating with the controller 120, e.g., via the injection control line 312 or a wireless connection.

The components of the tissue treatment system 100 may be integrated within a same enclosure, or may disparately located. For example, the fluid transfer unit 130 is shown within a same box as, indicating a same housing as, the one or more processors 602. It will be appreciated, however, that the fluid transfer unit 130 or other components of the controller 120 may be physically separated and remotely located from the one or more processors 602. For example, the contrast injector 310 may be integrated within the controller 120, e.g., as a portion of the fluid transfer unit 130. The contrast injector 310 can be integrated within a fluid cartridge of the fluid transfer unit 130. In such case, contrast injector 310 may include a syringe 405 that is prefilled with contrast medium. The contrast injector 310 may alternatively be mounted on a side of the controller 120. The clinician may fill a syringe 405 and load the syringe into the contrast injector 310 on a side of the controller 120. In another embodiment, the contrast injector 310 may be external to, and spaced away from, the controller 120. Such an embodiment is shown in FIG. 3. Wired or wireless electrical communication may be established between the one or more processors 602 and the integrated or remotely located components to perform the method described herein.

The contrast injector 310 is an example of a system component that may be spaced apart from the controller 120. Electrical communication between the processor 602 and the contrast injector 310 may be maintained through wireless or wired connections. For example, the contrast injector 310 can include a wireless chip within the electronics housing 403 to communicate wirelessly with a transceiver of the controller 120. In such an embodiment, the contrast injector 310 may include a rechargeable power source, given that the injection control line 312 that could otherwise power the contrast injector 310 may not be present.

When the contrast injector 310 is external to the controller 120, the contrast injector 310 may be located in a different sterility zone of the operating arena. For example, the controller 120 may be situated in a non-sterile zone of the arena, and the contrast injector 310 may be positioned within a sterile zone of the arena. The contrast injector 310 may nonetheless be controlled by the controller 120 to supply contrast medium to the guide catheter 301. In such an embodiment, the contrast injector 310 can have a high ingress rating to allow the unit to be sterilized. Alternatively, the contrast injector 310 may also be in the non-sterile zone, and contrast medium may be conveyed to the guide catheter 301 within the sterile zone through a longer length of injection tubing 316. It will be appreciated from the description above that system components may be physically spaced from each other and interconnected via electrical (wired or wireless) and/or fluid lines.

Having discussed the tissue treatment system 100, a method of using the tissue treatment system 100 to confirm apposition to and/or occlusion of the target vessel 200 is now described. The tissue treatment system 100 can ensure that pressure built up in the target vessel 200 behind the inflated balloon 112 does not exceed an inflation pressure of the balloon. Furthermore, the system can perform the method with minimal contrast medium, and without requiring substantive physical exertion by the clinician. Accordingly, the method may be performed easily and safely to confirm that the balloon 112 is properly inflated within the target vessel 200 prior to initiating sonication of the ablation target.

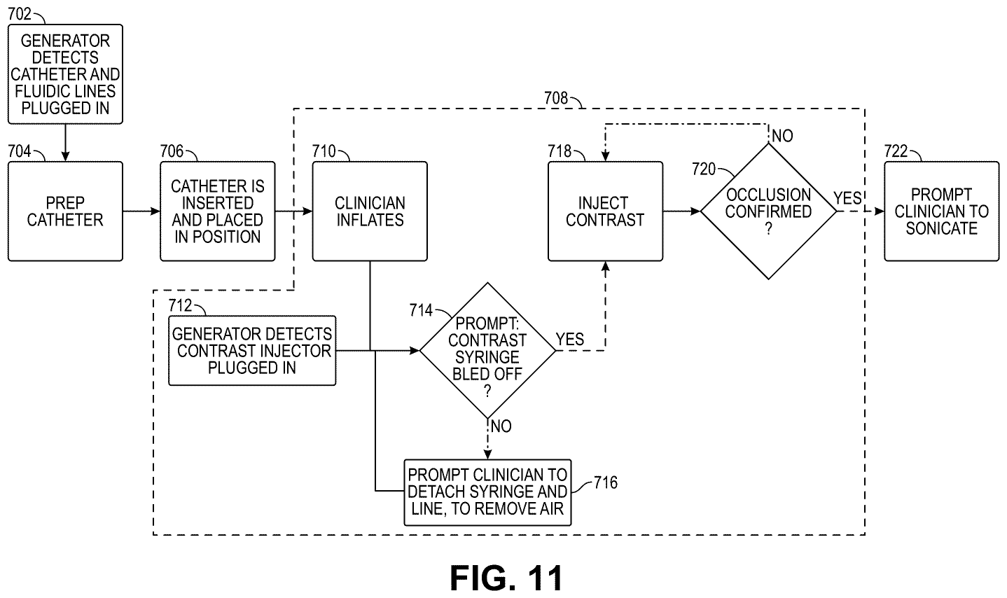

Referring to FIG. 11, a flowchart of a method of deploying a tissue treatment system is shown in accordance with an embodiment. At operation 702, the clinician can connect the tissue treatment catheter 102 and the fluidic lines to the controller 120. The controller 120 can detect that the catheter and the fluidic lines are connected, e.g., plugged in to the controller 120. At operation 704, the clinician can prepare the catheter for insertion into the target anatomy. Preparation of the catheter can include purging air from the balloon 112 and the catheter shaft 214. At operation 706, the prepped catheter is inserted into the target anatomy. The clinician can advance the tissue treatment catheter 102 through the target anatomy to position the balloon 112 adjacent to the ablation target, e.g., within the renal artery.

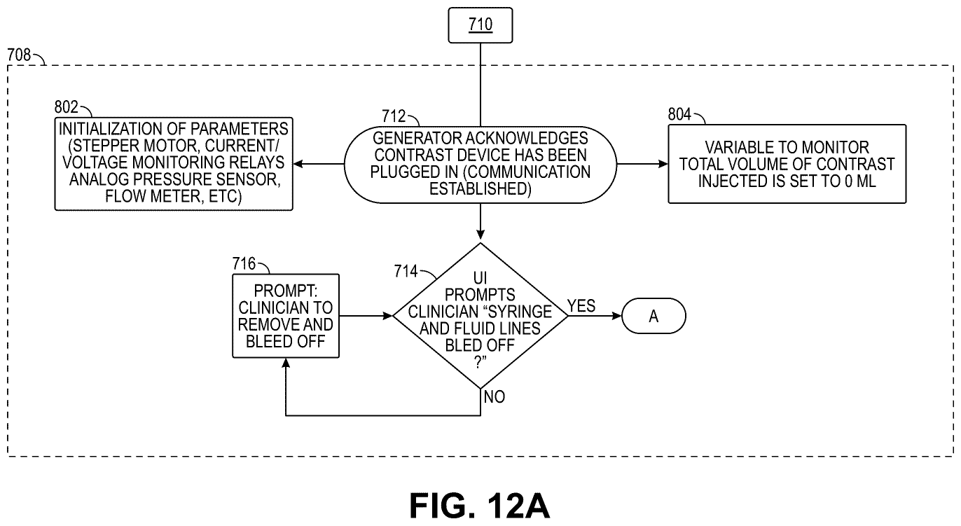

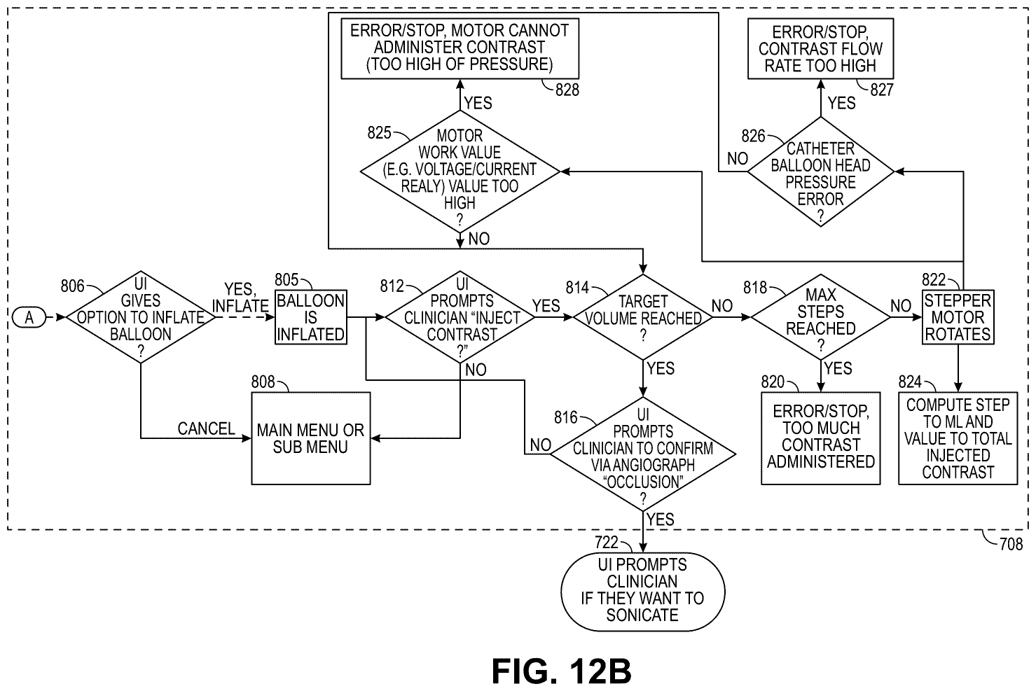

The operations enclosed by the dashed lines in FIG. 11 correspond to the operations of inflating the balloon 112 and administering contrast medium to confirm apposition and/or occlusion between the balloon 112 and the target vessel 200. Such operations are described at a high level in FIG. 11, and with further granularity in FIG. 12A. It will be appreciated that the operations are performed via interaction between the clinician and the controller 120. More particularly, the clinician can interact with the graphical user interface of the controller 120 to control the operations of the balloon inflation and contrast injection.

At operation 710, the clinician inflates the balloon 112. Balloon inflation may be initiated through a user interface element, as described below. When the balloon 112 is inflated, the contrast injector 310 may be used to inject contrast medium through the guide catheter 301 into the vessel space behind the inflated balloon 112.

Prior to inflating the balloon 112, the clinician can prepare the contrast injector 310. The clinician can fill the syringe 405 with contrast medium. Air can be bled from the syringe 405, and the injection lines can be purged. Bleeding of the syringe 405 and purging of the injection lines may be performed manually, or via an automated process performed by the tissue treatment system 100. The clinician may insert the syringe 405 into the contrast injector 310. For example, the pivotable caps may be moved to the open position, the syringe flanges can be inserted into the slots 502 of the drive heads 440, 441, and the caps can be closed and latched to secure the syringe 405 within contrast injector 310. When the syringe 405 is in place, contrast injection may be initiated.

At operation 712, the controller 120 can detect whether the syringe 405 is loaded into the contrast injector 310. Furthermore, the system can detect whether the contrast injector 310 is communicating with the controller 120, e.g., via the injection control line 312 or a wireless connection.

At operation 714, the tissue treatment system 100 can prompt the clinician to confirm whether air has been bled from the syringe 405. For example, in response to detecting that the injection control line 312 is plugged in, the user interface of the controller 120 can display a prompt requesting confirmation that the clinician has bled syringe 405 and/or purged injection tubing 316. At operation 716, if the clinician has overlooked the bleeding operation, the clinician can detach the syringe 405 and the injection tubing 316 to perform the bleeding operation and remove air from the lines.

At operation 718, when the clinician confirms that air is bled from the contrast injector subsystem, contrast injection can begin. The controller 120 can drive the drive motor 460 to advance the plunger 462 of the syringe 405 and expel contrast medium into the injection tubing 316 and through the guide catheter 301. The injected contrast can be delivered into the target vessel 200 to a space between a distal end of the guide catheter 301 and the inflated balloon 112. Injection of the contrast medium can be performed according to the suboperations described below with respect to FIG. 12.

At operation 720, the clinician can be prompted to confirm whether the inflated balloon 112 has occluded the target vessel 200. More particularly, in response to the contrast medium being injected, the user interface can display a prompt requesting that the clinician confirm whether occlusion has been achieved. The physician can view the target anatomy under fluoroscopy to assess whether contrast medium remains behind the inflated balloon 112 or flowed downstream. Stagnant contrast medium behind the inflated balloon 112 suggests that the target vessel is occluded, and downstream flow of the contrast medium suggests that the vessel is not occluded. The clinician can select appropriate user interface elements to make the confirmation.

At operation 722, when occlusion has been confirmed by the clinician, the tissue treatment system 100 can initiate sonication of the ablation target. More particularly, the system can, in response to receiving confirmation of occlusion, display a prompt on the user interface. The prompt can request confirmation about whether the clinician is ready to begin sonication. When the clinician confirms that sonication may begin, the controller 120 can control the energy delivery unit 134 to energize the ultrasound transducer 210. The ultrasound transducer 210 may then emit acoustic energy through the inflation fluid 111 to the ablation target.

Referring to FIGS. 12A-12B, flowcharts of a method of controlling injection of contrast medium by a tissue treatment system 100 is shown in accordance with an embodiment. The operations of FIGS. 12A-12B correspond to the operations grouped under 708 in FIG. 11. Referring to FIG. 12A, for example, after the clinician inflates the balloon 112 at operation 710, operation 712 can be performed by the tissue treatment system 100 to determine whether the contrast injector 310 is in electrical communication with the energy delivery unit 134 of the tissue treatment system 100. One or more processors 602 can detect and acknowledge that contrast injector 310 is plugged into, or has otherwise established communication with, the controller 120.

When the contrast injector 310 has established communication with the controller 120, the controller can initialize the contrast injector 310. At operation 802, the controller 120 can initialize parameters of the contrast injector 310. For example, the controller 120 can communicate with and initialize the drive motor 460, electrical monitoring relays within the electrical housing 403, sensors (such as the injection sensor 350), etc. At operation 804, the one or more processors 602 of the controller 120 may also set a value for a volume of contrast injected. More particularly, the one or more processors 602 may monitor the volume of contrast medium injected during the method, and at initialization, a value of the volume may be set to 0 mL.

After initialization, operations 714 and 716 can be performed as described above. More particularly, the controller 120 can prompt the clinician to confirm whether the fluidic volumes of the contrast injector 310 have been bled of air. In the event that they have not, the clinician is provided an opportunity to detach the contrast injector components to perform the bleeding operation. Bleeding air from the system reduces the likelihood of air being delivered into a vasculature of the patient. Accordingly, the confirmation operation enhances safety of the procedure.

Referring to FIG. 12B, at operation 806, which may be a sub-operation of operation 710, the tissue treatment system 100 provides an option to the clinician to begin balloon inflation. For example, the user interface may display a prompt requesting the clinician to input a command to begin inflation. In the event that the clinician selects to cancel inflation, at operation 808, the user interface may display a main menu or a submenu to allow the clinician to take action other than inflating the balloon 112. If, however, the clinician elects to inflate the balloon 112, the one or more processors 602 can receive the user input, and in response, can control the fluid transfer unit 130 to deliver inflation fluid 111 to the balloon 112. More particularly, at operation 810, the fluid transfer unit 130 of the tissue treatment system 100 delivers the inflation fluid 111 at an inflation pressure to the balloon 112. The balloon 112 can inflate.

At operation 812, which may be a sub-operation of operation 718, the tissue treatment system 100 provides an option to the clinician to inject contrast. For example, the user interface may display a prompt requesting the clinician to input a command to begin contrast injection. The prompt may include user elements indicating “contrast puff” and “cancel,” for example. In the event that the clinician selects not to inject contrast, e.g., by selecting “cancel,” at operation 808, the user interface may display a main menu or a submenu to allow the clinician to take action other than inflating the balloon 112. If, however, the clinician elects to inject contrast medium, e.g., by selecting “contrast puff,” the one or more processors 602 can receive the user input and, in response, can control contrast injector 310 to deliver contrast medium. The contrast injector 310 can deliver the contrast medium over a period of time to the guide catheter 301 and into the target vessel 200. More particularly, upon receiving the user input requesting injection of the contrast medium, the contrast injector 310 of the tissue treatment system 100 can inject contrast medium at an injection pressure into the vessel 200 containing the balloon 112.

In an embodiment, contrast injection may be controlled in part based on whether a target volume of contrast medium has been delivered into the vessel 200. At operation 814, the one or more processors 602 can determine whether the target volume of contrast has been reached. The delivered volume of contrast may be determined based on motor control. For example, the one or more processors 602 may determine an amount of movement performed by the drive motor 460, e.g., a number of steps made by a stepper motor, and calculate an injection volume based on such information. Injection of the contrast medium may be stopped in response to the calculated volume being equal to the target volume of contrast medium. The target volume of contrast medium can be a volume of contrast medium required to visualize the target anatomy under fluoroscopy. When the delivered volume equals the target volume of contrast medium, the controller 120 can determine sufficient contrast medium has been delivered to permit the clinician to assess occlusion of the vessel 200. In response to such determination, at operation 816, the system can prompt the clinician to confirm whether the vessel 200 is occluded. For example, the user interface can display a prompt to the clinician requesting confirmation via angiograph of whether the target vessel 200 is occluded.

If the clinician confirms that vessel occlusion has been achieved, the system can progress to operation 722 at which the sonication operation may be initiated, as described above. Alternatively, if the clinician confirms that vessel occlusion has not occurred, the system may revert to operation 812 to enable the clinician to initiate an additional contrast puff.

When contrast injection is underway, at operation 814, and the target volume has not been reached, processing can advance to operation 818. At operation 818, the one or more processors 602 can determine whether a predetermined maximum volume of contrast medium has been delivered into the vessel 200. The predetermined maximum volume of contrast medium can be a limit set at a safe volume of contrast medium to be administered to the patient. When the delivered volume equals the predetermined maximum volume of contrast medium, the controller 120 can determine that a safety limit of contrast medium being delivered into the vessel 200 has been reached. In response to such determination, at operation 820, the system can stop injection of the contrast medium. For example, the tissue treatment system 100 can generate an error message, which may be displayed on the user interface of the controller 120. The error message can indicate that a maximum permitted amount of contrast has been administered, and the process can end.

At operation 822, when the predetermined maximum volume of contrast medium has not been delivered into the vessel 200, the drive mechanism of the contrast injector 310 can actuate the plunger 462 to expel contrast medium from the syringe 405. For example, the drive motor 460 can rotate to move the drive shaft 436 that advances the syringe plunger cap 430 forward. The plunger 462 can push contrast medium out of the syringe body 424.

At operation 824, the one or more processors 602 can determine and/or update the value for the injected volume of contrast medium. For example, the one or more processors 602 can compute, based on a number of steps of the drive (e.g., stepper) motor 460, the volume in mL of contrast medium injected into the target vessel 200. The calculated volume can be added to the previously recorded value, e.g., zero, and stored for later comparison to the target injection volume and/or the predetermined maximum volume at operations 814 and 818.

During injection of the contrast medium, the controller 120 can monitor the inflation pressure of the inflation fluid 111 in the balloon 112 and/or the injection pressure of contrast medium. The one or more processors 602 of the controller 120 can monitor the inflation pressure via communication with the fluid transfer unit 130. More particularly, the fluid transfer unit 130 can include one or more sensors to detect the pressure of the inflation fluid 111 within the balloon 112 and/or the inflation tubing 138, and the detected pressure can be relayed to the one or more processors 602. Accordingly, the one or more processors 602 can determine the inflation pressure. Similarly, one or more processors 602 of the controller 120 can monitor the injection pressure via communication with the contrast injector 310. More particularly, contrast injector 310 can include the injection sensor 350 to detect pressure of the contrast medium within the injection tubing 316 and/or the space within the vessel 200 between the distal end of the guide catheter 301 and the inflated balloon 112. The detected pressure can be relayed to the one or more processors 602. Accordingly, the one or more processors 602 can determine the injection pressure.

The injection pressure, instead of or in addition to be, sensed by the injection sensor 350, may be determined based on electrical parameters of the driving mechanism. For example, at operation 825, the one or more processors 602 can determine the injection pressure based on a motor work value of the drive motor 460 of the contrast injector 310. The motor work value can be determined based on a voltage and/or a current of electricity supplied to the drive motor 460 during contrast injection. The electrical parameters can be used to calculate the motor work value, which corresponds to the work required to advance the plunger 462 within the barrel of the syringe 405. Such work correlates to the pressure of the contrast medium, and thus, may be used to determine the injection pressure.

Injection of the contrast medium may be controlled based on the inflation pressure and/or the injection pressure. In an embodiment, at operation 826, injection of the contrast medium is stopped based on the inflation pressure. For example, the injection may be stopped based on the inflation pressure being equal to an inflation pressure limit. The inflation pressure limit can correspond to an inflation pressure that results from compression of the balloon 112 by the injected contrast medium. For example, an initial inflation pressure to inflate the balloon 112 may have a first value. As contrast medium is flowed into the target vessel 200 behind the inflated balloon 112, the contrast medium can press on the inflated balloon wall. Such compression can result from a high flow rate, and can increase the inflation pressure of the inflation fluid 111 within the balloon 112 to a second, higher value. When the contrast medium overcomes and collapses the inflated balloon 112, for example, when the flow rate is too high, the inflation pressure will be above the inflation pressure limit. More particularly, the inflation pressure limit can be set to a predetermined pressure at which the inflated balloon 112 is known to have not been collapsed by high flow of the contrast medium. Stopping the contrast medium injection when the inflation pressure is equal to the inflation pressure limit can reduce the likelihood that the balloon 112 will collapse and cause the loss of occlusion. At operation 827, when the contrast injection is stopped based on the inflation pressure, the system can generate an error message indicating that the contrast flow rate is too high. More particularly, the one or more processors 602 can cause the user interface to present the error message to the clinician to indicate that the procedure has ended due to the contrast flow rates being too high.

Injection of the contrast medium can be stopped based on the injection pressure. For example, the injection may be stopped when the injection pressure is equal to the injection pressure limit. The injection pressure limit can correspond to an injection pressure at which the contrast medium can collapse the balloon 112. For example, when the injection pressure exceeds a predetermined value, the contrast medium can press inward on the balloon 112, causing the balloon 112 to collapse, and losing the contrast medium to downstream flow around the balloon 112.

In an embodiment, the injection pressure limit may be equal to the inflation pressure. More particularly, injection of the contrast medium may be stopped based on the injection pressure being equal to the inflation pressure. When the injection pressure is higher than the inflation pressure, the compressive force applied by the contrast medium on the balloon 112 may exceed the expansion force applied by the balloon 112 on the contrast medium and/or vessel wall 212. The balloon 112 may collapse as a result. Accordingly, injection of the contrast medium can be stopped prior to collapse when the injection pressure is equal to the inflation pressure.

At operation 828, when the contrast injection is stopped based on the injection pressure, the system can generate an error message indicating that the injection pressure is too high. More particularly, the one or more processors 602 can cause the user interface to present the error message to the clinician to indicate that the procedure has ended due to the contrast medium having a pressure that could potentially collapse the balloon 112.

When contrast medium injection is continued, e.g., when both the inflation pressure and the injection pressure are within operational limits, the one or more processors 602 can revert to operation 814. At operation 814, the system can determine whether the target volume is reached. Injection of the contrast medium may be stopped in response to the target volume of contrast medium being delivered into the vessel 200. As described above, when the target volume is reached, a prompt may be displayed requesting confirmation of whether the vessel 200 is occluded.

The clinician can respond to the displayed prompt by confirming that the vessel 200 is occluded or by confirming that the vessel 200 is not occluded. In an embodiment, the system receives the user input confirming that the vessel 200 is not occluded. In response to the user input confirming that the vessel 200 is not occluded, a resume control element may be displayed by the user interface. The resume control element can be a user element selectable by the clinician to resume contrast injection. For example, the resume control element may be displayed at operation 812, and upon selection, the tissue treatment system 100 can inject contrast progressing through the operations described above. By contrast, user input may confirm that the vessel 200 is occluded. In such case, the tissue treatment system 100 can advance through the method to operation 722.

At operation 722, as described above, the user may be given the option to begin sonication of the ablation target. More particularly, in response of the user input confirming that the vessel 200 is occluded, the controller 120 can display an initiate control element on the user interface. The initiate control element can be a user element selectable to initiate delivery of energy to the transducer 210. More particularly, energy can be delivered to the ultrasound transducer by the energy delivery unit 134 of the tissue treatment system 100. The energized transducer, which is contained within the balloon 112, can emit acoustic energy through the inflation fluid 111 to the ablation target to provide the desired therapeutic effect. The method, including injection of contrast medium to confirm occlusion of the target vessel 200 by the inflated balloon 112, may progress to an end.

Referring to FIG. 13, a perspective view of a catheter-based intraluminal device of a treatment system is shown in accordance with an embodiment. The catheter-based intraluminal device of a treatment system 100 can include a catheter 101 having an elongated catheter body extending from a proximal catheter end 1350 to a distal catheter end 104. An expandable member 106, such as a balloon 112, may be mounted on the catheter 101 at the distal catheter end 104. One or more energy transducers 108, such as ultrasound transducers 210, may be positioned within the expandable member 106. The expandable member 106 can be adapted to inflate within a target anatomy, e.g., a renal artery, and the energy transducer 108 can be adapted to deliver ablation energy, e.g., ultrasound energy, to the target anatomy during a medical procedure, e.g., a renal denervation procedure.

The catheter 101 can include one or more lumens, such as: fluid lumens to deliver an inflation/cooling fluid to the expandable member 106, electrical cable passageways containing electrical cables to deliver energy to the transducer 108, guidewire lumens for exchanging guidewires, etc. The lumen(s) may be connected to corresponding connectors at the proximal catheter end 1350. For example, the fluid lumens may connect to one or more catheter fluid ports 1352, which receive inflation/cooling fluid from a fluid transfer cartridge of the treatment system 100, as described below. Similarly, the electrical cables can connect to an external connector 1352, which receives energy from a generator of the treatment system 100, as described below.

Referring to FIG. 14, a front perspective view of a generator and a fluid transfer cartridge of a treatment system is shown in accordance with an embodiment. The treatment system 100 includes a control unit that connects to the catheter 101 to regulate the inflation of the balloon 106 with inflation/cooling fluid and to manage the delivery of ultrasound energy to the transducer 108. In an embodiment, the control unit includes a generator 202 to generate the ultrasound energy, and a fluid transfer cartridge 204 to transfer cooling fluid to and from the balloon 106 through one or more fluid conduits 206. For example, a fluid conduit 206A may transfer cooling fluid between a fluid reservoir, e.g., an intravascular fluid bag, and the fluid transfer cartridge 204. Similarly, a fluid conduit 206B can transfer cooling fluid between the fluid transfer cartridge 204 and the catheter 101. The control unit includes several other components, some of which are described below, to facilitate the energy and fluid transfer functions. Such components can include a display 208 to present procedural information to a user. Furthermore, the control unit can include one or more processors (not shown) configured to execute instructions stored in a memory device (not shown) to cause the treatment system 100 to perform various operations of the medical procedure, as described below.

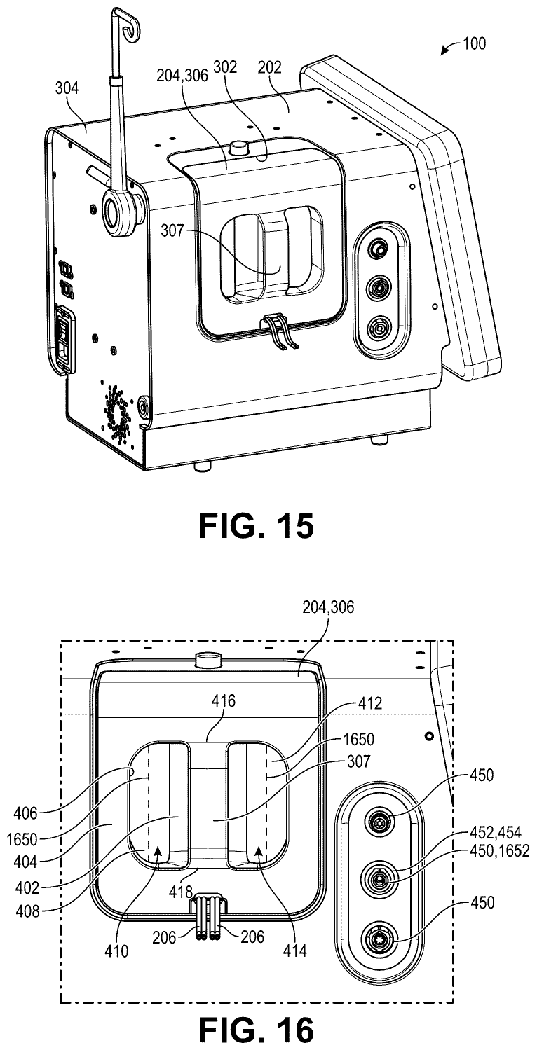

Referring to FIG. 15, a rear perspective view of a generator and a fluid transfer cartridge of a treatment system is shown in accordance with an embodiment. The generator 202 of the treatment system 100 can have a cartridge receptacle 302 shaped and sized to receive the fluid transfer cartridge 204. More particularly, the generator 202 can include a generator housing 304 having the cartridge receptacle 302 configured to receive the fluid transfer cartridge 204. The generator housing 304 can include an external wall having a shape, e.g., a box-like envelope, and the cartridge receptacle 302 may be a recessed region extending into the shape. The fluid transfer cartridge 204 can include a cartridge shell 306, which may fill the cartridge receptacle 302 of the generator 202. More particularly, the cartridge shell 306 can include an external wall having a shape that merges smoothly with the generator housing wall to complete the envelope of the generator 202. For example, the fluid transfer cartridge 204 and the generator 202 can combine to form the box-like envelope. In such case, the external, outward-facing surfaces of the fluid transfer cartridge 204 and generator 202 can be parallel and coplanar at a seam where the components meets such that the form factor of the combined components transitions smoothly, e.g., non-stepped, at the transition between the generator wall and the fluid transfer cartridge wall.

In an embodiment, the fluid transfer cartridge 204 includes a handle 307 that the user can hold when mounting or dismounting the cartridge from the generator 202. The handle 307 can include a curved, ergonomic shape that is easily grasped. Accordingly, the user can carry the fluid transfer cartridge 204 by the handle 307, and insert the fluid transfer cartridge 204 into the cartridge receptacle 302 to engage the components in the compact form factor having an external wall that extends continuously over the generator housing 304 and the cartridge shell 306.

In combination with the generator 202, the fluid transfer cartridge 204 can be used to drive fluid into the catheter 101 using one or more syringes. More particularly, the fluid transfer cartridge 204 can include one or more syringes that pump fluid to and from the balloon 106. As described below, each syringe can include a respective syringe piston disposed within a respective syringe barrel. Movement of the syringe piston relative to the syringe barrel can draw cooling fluid into the syringe or expel cooling fluid out of the syringe. The fluid can be transferred to and from the fluid transfer cartridge 204 through fluid conduits 206 that may be connected to a catheter 101, a fluid reservoir, or another fluid vessel external to the fluid transfer cartridge 204.

Referring to FIG. 16, a perspective view of a cartridge receiving portion of a generator and a fluid transfer cartridge of a treatment system is shown in accordance with an embodiment. The cartridge shell 306 of the fluid transfer cartridge 204 can define a cartridge cavity 402 within which the one or more syringes are disposed. More particularly, the cartridge shell 306 can define the cartridge cavity 402 between a front face 404 of the cartridge shell 306, and a rear face 407 (FIG. 20) of the cartridge shell 306. A space between the front and rear faces 404, 407 of the cartridge shell 306 can contain the syringe(s). Accordingly, the syringes (including the syringe shafts of the syringes) may be stored internal to the cartridge shell 306, rather than being exposed outward from the shell. Thus, the overall form factor of the fluid transfer cartridge 204 may be compacted. The cartridge cavity 402 may further be defined between an upper face and a bottom face of the cartridge shell 306.

The one or more syringes of the fluid transfer cartridge 204 can be disposed within the cartridge cavity 402 parallel to the handle 307. For example, the handle 307 can extend from the front face 404 of the cartridge shell 306 over an opening that opens into the cartridge cavity 402. The opening can be a window 406 or a portal that exposes an internal volume of the cartridge cavity 402 to view from the surrounding environment. More particularly, the front face 404 can include the opening that visibly exposes the cartridge cavity 402 to view by the user. The handle 307 can extend vertically from an upper end 416 of the opening to a lower end 418 of the opening. Accordingly, the handle 307 can curve outward from the front face 404 above the opening, and vertically downward over the opening, to terminate at the front face 404 below the opening. Likewise, the syringe(s) can extend vertically through the cartridge cavity 402 such that the syringe barrels are visibly exposed through the opening.

In an embodiment, the fluid transfer cartridge 204 includes a first syringe barrel 408 within the cartridge cavity 402 and visibly exposed through the opening on a first side 410 of the handle 307. Similarly, the fluid transfer cartridge 204 can include a second syringe barrel 412 disposed within the cartridge cavity 402 and visibly exposed through the opening on a second side 414 of the handle 307. The syringes, like the handle 307, may extend vertically, e.g., within the cartridge cavity 402. More particularly, the first syringe barrel 408 and the second syringe barrel 412 can have respective syringe axes 420 that extend vertically, e.g., within the cartridge cavity 402. The syringe axes 420 can be central axes of the syringe barrels. For example, the syringe barrels may be cylindrical and may extend along the syringe axis 1650 in a vertical direction. Accordingly, the handle 307 may be easily grasped from the front of the fluid transfer cartridge 204, however, the syringes may remain exposed to view through the opening in the front face 404. Thus, the fluid transfer cartridge 204 is easy to handle, easy to view, and has a compact form factor that meshes with the generator 202.

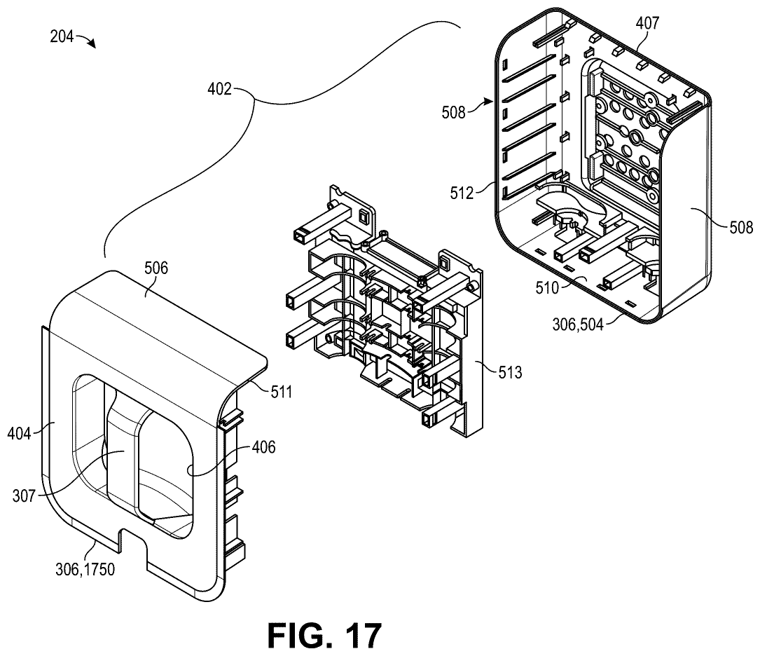

Referring to FIG. 17, an exploded view of a cartridge shell of a fluid transfer cartridge is shown in accordance with an embodiment. The cartridge shell 306 provides an external envelope of the fluid transfer cartridge 204 and can have a variety of geometries.

The fluid transfer cartridge 204 can include several portions that are snap-fit or otherwise fastened together. The cartridge shell 306 may include a handle front plate 1750 and a back plate 504. The handle front plate 1750 can include the handle 307, the front face 404 of the shell, and an upper face 506 of the shell. Similarly, the back plate 504 can include the rear face 407 of the shell, several sidewalls 508 laterally outward from the cartridge cavity 402, and a bottom face 510 of the shell. When combined, the handle front plate 1750 and the back plate 504 can define the cartridge cavity 402 centrally located between the various walls and faces. As described above, the cartridge cavity 402 may nonetheless be visibly exposed through the opening 406 in the handle front plate 1750.

The front face 404 can include a front face perimeter 511. The front face perimeter 511 can be an outer edge of the front face 404. The front face perimeter 511 can have curved and straight edges that combine to form an outline of the front face 404. Similarly, the rear face 407 can include a rear face perimeter 512. The rear face perimeter 512 can have curved and straight edges that combine to form an outline of the rear face 407. In an embodiment, the front face perimeter 511 has a same profile as the rear face perimeter 512. More particularly, an outline of the front face perimeter 511 can conform to an outline of the rear face perimeter 512 such that, when the front face perimeter 511 is engaged to the rear face perimeter 512, the perimeters effectively seal or contact each other. When the perimeters are in contact, the cartridge cavity 402 can be enclosed within the cartridge shell 306. The enclosed cartridge cavity 402 is defined between the front face 404 and the rear face 407.

When snapped or otherwise fit together, the handle front plate 1750 and the back plate 504 can contain, within the cartridge cavity 402, one or more components to provide fluid transfer functionality. For example, the fluid transfer cartridge 204 can include a syringe holder 513 to hold the syringes within the cartridge cavity 402. The syringe holder 513 can stabilize the syringes during fluid delivery, as described below. The fluid transfer cartridge 204 can also include a manifold, tubing, electronics, etc. (not shown) that facilitate the movement of fluid from the syringes to the fluid conduits 206 and the catheter 101. The internal components of the fluid transfer cartridge 204 can be constrained within the internal space of the cartridge shell 306 and can interact mechanically and/or electrically with each other and with the generator 202 to perform the fluid transfer function of delivering inflation/cooling fluid to the catheter fluid ports 1352 of the catheter 101.

In an embodiment, the syringe holder 513, which is mounted within the cartridge cavity 402, limits movement of the syringe. The syringe may have a plunger that is driven axially within the syringe barrel 408 during operation. The plunger may rotate about the syringe axis 1650, and thus, may impart some rotational loads to the syringe. The syringe holder 513 can resist rotation of the barrel that may otherwise be caused by the plunger. The syringe holder 513 can include one or more stop features. The stop features can be ribs, protrusions, or other features formed in the syringe holder 513. Stop features may remain fixed relative to the front and rear faces 404, 407 of the fluid transfer cartridge 204 when the cartridge is assembled. Furthermore, when assembled, a portion of the syringe barrel 408, such as a tab extending laterally outward from the cylindrical syringe barrel 408, can engage the stop feature. For example, the syringe barrel 408 can have finger tabs that are fit into a corresponding recess of the syringe holder 513. The finger tabs may be nestled within the recess between several ridges such that the syringe holder 513 mechanically interferes with movement of the tabs. Accordingly, the stop feature engages the interference feature of the syringe barrel 408 such that rotation of the syringe barrel 408 relative to the cartridge shell 306 is limited.

Referring to FIG. 18, a sectional view of an end-lit syringe of a fluid transfer cartridge is shown in accordance with an embodiment. The fluid transfer cartridge 204 can include one or more light sources 602 within the cartridge cavity 402. For example, the light source(s) 602 can be light emitting diodes. As illustrated, two light sources 602 may be symmetrically located on each side of the syringe axis 1650. Alternatively, three or more light sources 602 could be symmetrically disposed around the syringe axis 1650. More particularly, when viewed from above, an angle between the light sources 602 may be 360° divided by the number of light sources 602 (e.g., 120° between each of the three light sources 602 when viewed along the syringe axis 1650).

The light source 1850 can illuminate one or more of the syringes such that the syringe barrel 408 and/or a cooling fluid 603 within the syringe can be seen by the user. More particularly, the light source 1850 can be within the cartridge cavity 402, and can be directed through the syringe barrel 408 to illuminate the syringe barrel 408 contents and/or the wall of the syringe barrel 408. When the light source 1850 is activated, the user may look through the opening/window 406 in the fluid transfer cartridge 204 to see the syringe and cooling fluid operation. It will be appreciated that, especially when viewed within a dark procedure room, the light source 1850 allows for easier monitoring of fluid transfer and troubleshooting of cooling fluid issues.

In an embodiment, the light source 1850 is directed through the syringe barrel 408. For example, the syringe can be end-lit by the light source 1850. Each of the one or more light sources 602 can direct light 606 into a respective light guide 605. The light guides 605 can include cylindrical transparent columns that act as light pipes to convey light 606 from the light source 1850 to a distal (upper) end of the syringe. More particularly, the light source 1850 may be directed through an end face 1852 of the syringe barrel 408. The end face 1852 can be a section of the barrel that is angled or tapered toward the syringe axis 1650 from the substantially cylindrical side wall of the syringe barrel 408. Accordingly, an axis directed normal to the end face 1852 outer surface may form an angle with the syringe axis 1650 that is less than an angle formed between the syringe axis 1650 and an axis normal to the barrel side wall.

The syringe may be side-lit by the light source 1850. For example, rather than shining longitudinally into the syringe, the light source 1850 may be directed in a transverse or radial direction relative to the syringe axis 1650. Accordingly, the light 606 can illuminate the syringe barrel 408 and its contents from an end, side, or rear of the cartridge cavity 402.

In certain embodiments, the light source 1850 may be directed through the syringe barrel 408 in order to help with assembly.

Light 606 can be emitted in a direction of the syringe axis 1650. Accordingly, a portion of the light 606 can transmit through the end face 1852 into the syringe barrel 408 and the fluid contained within. Furthermore, a portion of the light 606 can transmit into a wall of the syringe barrel 408. The light 606 can propagate along the wall creating a light pipe effect. The end-lit syringe barrel 408 can therefore provide good contrast against the fluid and mechanical components, e.g., a stopper 608, of the syringe. Accordingly, the light source 1850 makes it easier to see syringe movement and otherwise visualize the function of the syringe during the medical procedure.

The light source 1850 may have visible characteristics that are ergonomic. In an embodiment, the light source 1850 emits a blue light. More particularly, a wavelength of the light 606 emitted by the light source 1850 can be in the visible blue range. In a dark procedure room, the blue light can have a cool and calming effect. Furthermore, the blue light can have an intensity that is not distracting to the user, yet adequately lights the cooling fluid 603 for accurate monitoring.

In addition to illuminating the syringe components, the light source 1850 may have visible characteristics that provide cues and visual feedback to the user. The control unit includes one or more processors configured to activate the light source 1850 based on inputs from one or more sensors. In an embodiment, the light source 1850 emits a first color of light 606 when the syringe barrel 408 is filled with a first volume of fluid. The first volume of fluid can be detected using flow or other fluid sensors. The one or more processors may receive a volume signal from the sensors indicating that the first volume of fluid is contained within the syringe barrel 408. In response to the volume signal, the one or more processors may cause the light source 1850 to emit the first color of light, e.g., orange. The control unit may be configured such that the light source 1850 emits a second color of light 606 when the syringe barrel 408 is filled with a second volume of fluid. For example, in response to detecting the second volume of fluid, the one or more processors may cause the light source 1850 to emit the second color of light, e.g., blue. The user may therefore readily recognize whether the syringe is partially filled, based on the presence of orange light, or fully filled, based on the presence of blue light. Of course, the light colors may indicate different levels of fill, or amounts of fluid in the syringe barrel 408, and this example is not limiting. Similarly, an intensity or another light characteristic (other than color) may be altered based on fill level, and thus, the example of color changing is non-limiting.

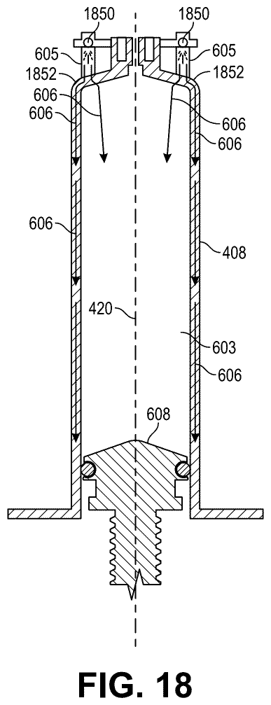

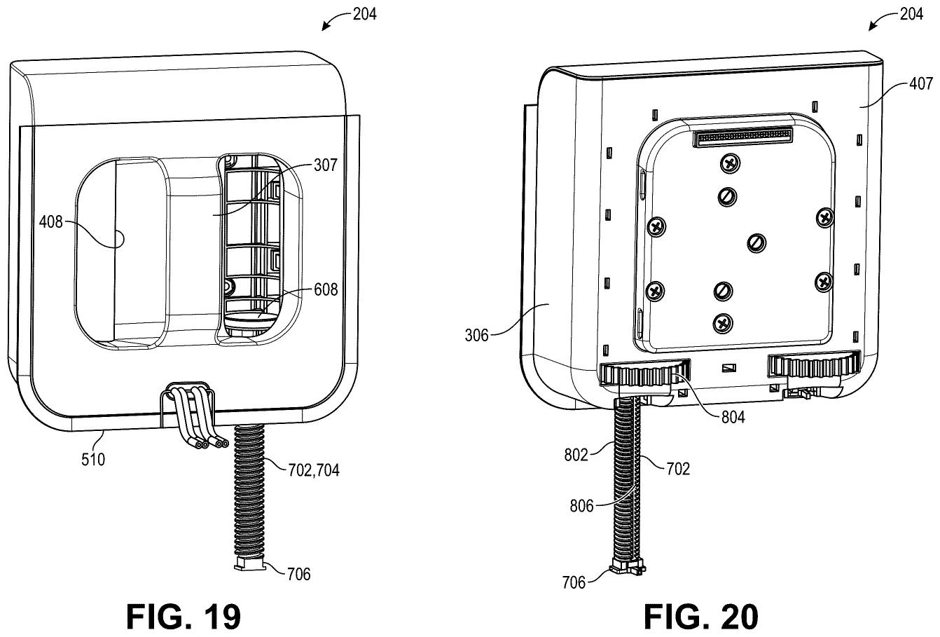

Referring to FIG. 19, a front perspective view of a fluid transfer cartridge is shown in accordance with an embodiment. As described above, each syringe of the fluid transfer cartridge 204 can include a syringe piston 702 disposed within the syringe barrel 408. The leftward syringe barrel is illustrated as opaque, and the rightward syringe barrel is illustrated as transparent to expose the stopper 608 within the syringe cavity. An opacity of the syringe barrel may vary.

The syringe piston 702 can include the stopper 608, which may be a rubber stopper capable of imparting friction and spinning the syringe barrel 408 if not otherwise resisted by the syringe holder 513. The syringe piston 702 may also include a shaft 704 extending from the stopper 608 within the syringe barrel 408 to a shaft end 706 outside of the syringe barrel 408. As described below, the shaft end 706 can include an element to trigger a position sensor. For example, the element can include a magnet to trigger a magnetic sensor, or an optical feature, e.g., a tab, prong, flag, etc., to trigger an optical sensor. Regardless of a position of the stopper 608 within the syringe barrel 408, the shaft end 706 may be outside of the syringe barrel 408. The shaft end 706, however, may have several positions, and at least one of the positions may be inside of the cartridge cavity 402. For example, a bottom surface of the shaft end 706 can be flush with the bottom face 510 of the cartridge shell 306.

In an embodiment, the shaft end 706 is disposed within the cartridge cavity 402 when the stopper 608 is at a home position in the syringe barrel 408. Alternatively, the shaft end 706 may be flush with the bottom face 510 of the cartridge shell 306 when the stopper 608 is at the home position. In either case, the shaft end 706 may not be external to the envelope defined by the cartridge shell 306 when the stopper 608 is at the home position. The home position may be an uppermost position of the stopper 608, or a position nearest to the end face 1852 of the syringe. In the home position, the shaft end 706 may be near but outside of the syringe barrel 408. More particularly, the shaft end 706 may have a vertical position between a proximal end of the syringe barrel 408 and the bottom face 510 of the fluid transfer cartridge 204. In the home position, the syringe components are entirely contained within the cartridge cavity 402. Accordingly, the fluid transfer cartridge 204 can have a compact form factor defined by the external surfaces of the cartridge shell 306, and without additional clearances required by the syringe or syringe components.

The fluid transfer cartridge 204 can be shipped with the syringe piston 702 in the home position. More particularly, prior to filling the syringe with the cooling fluid 603 (during shipment or upon initial mounting of the fluid transfer cartridge 204 on the generator 202), the stopper 608 can be at the home position. The package size may therefore be minimized, as compared to shipping the fluid transfer cartridge 204 with the syringe shaft 704 exposed from the cartridge shell 306, to reduce packaging requirements. Furthermore, the fluid transfer cartridge 204 may take up less space within the procedure room because the syringe and syringe components are not exposed outwardly from the cartridge. That is, the ability to move the shaft 704 entirely into the cartridge cavity 402 to contain the syringe entirely within the fluid transfer cartridge 204 can reduce an overall form factor of the fluid transfer cartridge 204. The reduced form factor makes the cartridge more compact for shipping and/or use.

In contrast to the home position, the shaft end 706 may be disposed outside of the cartridge cavity 402 when the stopper 608 is at an end position in the syringe barrel 408. The end position may be a lowermost position, or a position farthest from the end face 1852 of the syringe within the syringe barrel. In the end position, the shaft end 706 may be outside of the syringe barrel 408 and the cartridge cavity 402. More particularly, the shaft end 706 can be exposed below the bottom face 510 of the fluid transfer cartridge 204, as shown in FIG. 19.

Referring to FIG. 20, a rear perspective view of a fluid transfer cartridge is shown in accordance with an embodiment. The fluid transfer cartridge 204 includes a drive mechanism to advance the syringe piston 702 relative to the syringe barrel 408. In an embodiment, the shaft 704 of the syringe piston 702 includes an external thread 802 extending along an outer surface of the shaft 704 between the stopper 608 and shaft end 706. The fluid transfer cartridge 204 can also include a gear 804 mounted on the cartridge shell 306. The gear 804 can include an internal thread engaging the external thread 802 of the syringe shaft 704. Accordingly, as the gear 804 rotates, e.g., when driven by a motor of the generator 202, the internal thread of the gear 804 can drive the external thread 802 of the shaft 704 in an axial direction. Thus, the shaft 704 can be driven upward toward the home position and/or downward toward the end position. As the shaft 704 is driven downward, the stopper 608 can move away from the end face 1852 of the syringe barrel 408 to draw fluid into the syringe. By contrast, as the shaft 704 is driven upward, the stopper 608 can move toward the end face 1852 to expel fluid from the syringe barrel 408.

A rotational frictional load can be applied to the syringe shaft 704 by the gear 804 as the gear drives the syringe piston 702 in the direction of the syringe axis 1650. Similar to the stabilizing effect that the syringe holder 513 has on the syringe barrel 408, the fluid transfer cartridge 204 may also include a feature to stabilize the syringe piston 702. In an embodiment, the shaft 704 of the syringe piston 702 includes a piston notch 806 extending longitudinally between the stopper 608 and the shaft end 706. The longitudinal piston notch 806 can receive a prong (not shown) that extends from the cartridge shell 306 and/or the syringe holder 513. For example, the prong may be built into the chassis of the cartridge to fix the prong relative to the cartridge housing. As the gear 804 rotates relative to the shaft 704, it may impart a frictional load to the external threads 802 of the shaft 704. To ensure that the shaft 704 does not rotate relative to the gear 804, the prong can slide within the piston notch 806 and interfere with the notch walls to resist and limit rotation of the shaft 704 relative to the gear 804. The rotational movement is thereby converted into translational movement along the syringe axis 1650. More particularly, the prong can limit the rotation of the syringe piston 702 and constrain movement of the stopper 608 to be axial, with minimal rotation.

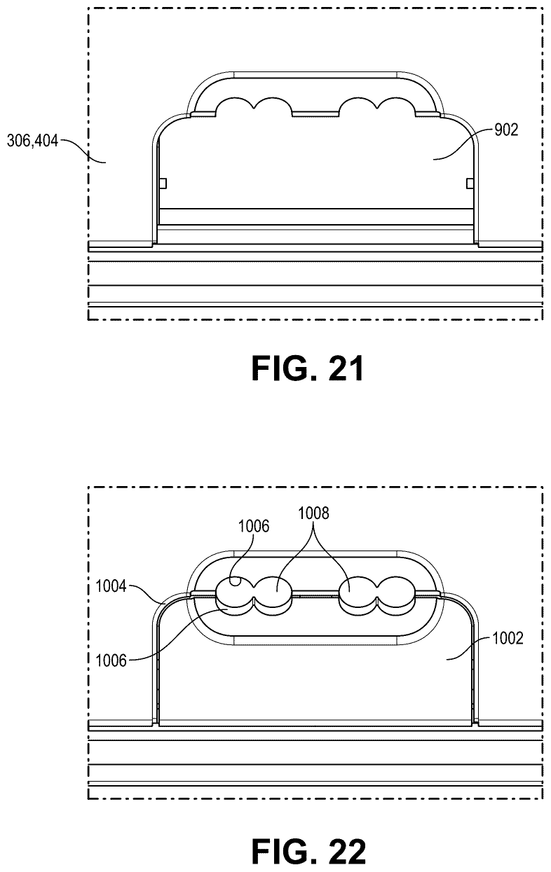

Referring to FIG. 21, a front view of a conduit routing port of a fluid transfer cartridge is shown in accordance with an embodiment. As described above, the components of the fluid transfer cartridge 204 may be fastened in various manners, including snap-fit connections. In an embodiment, the cartridge enclosure may include a conduit routing region through which the fluid conduits 206 may be routed. More particularly, the cartridge shell 306 can include a cartridge routing opening 902 in the front face 404. The cartridge routing opening 902 can improve manufacturability of the fluid transfer cartridge 204 by providing a cutout through which the fluid conduits 206, which may have substantial length, may be lodged into and/or routed through the fluid transfer cartridge 204. The cartridge routing opening 902 can be a cutout formed in the front face 404 along a lower edge of the cartridge. More particularly, the opening can have a lower edge that extends along the corner of the cartridge between the front face 404 and the bottom face 510 of the fluid transfer cartridge 204.

Referring to FIG. 22, a front view of a conduit routing plate mounted in a conduit routing opening of a fluid transfer cartridge is shown in accordance with an embodiment. The cartridge shell 306 can include a conduit routing plate 1002 that engages the front face 404 along an edge 1004. The edge 1004 can be a perimeter of the conduit routing opening. In an embodiment, the conduit routing plate 1002 and a portion of the front face 404 extending along the conduit routing opening can include respective notches 1006 at the edge 1004. For example, the notches 1006 in the front face 404 can be semicircular notches 1006, and the notches 1006 in a conduit routing plate 1002 may also be semicircular notches 1006. The conduit routing plate 1002 may be snap fit into (or otherwise fastened to) the conduit routing opening to occlude the opening and to form a hole through which the fluid conduit 206 may be routed. For example, the partial cutouts in the enclosure can combine to form conduit ports for the fluid conduits 206. In the case of semicircular notches 1006, the notches can combine to form a circular conduit routing port 1008 in the front face 404 of the assembled fluid transfer cartridge 204. Prior to snapping the conduit routing plate 1002 into the opening, a predetermined length of the fluid conduits 206 may be left exposed outside of the cartridge. When the conduit routing plate 1002 is snapped into the opening, the edge 1004 of the plate and the cartridge shell 306 can clamp around the fluid conduit 206 to hold the fluid conduit 206 in place. The ability to easily determine a length of conduit to insert into the cavity through the opening, and a length of conduit to extend outside of the opening (the lengths being separated by the plate that is snap fit into the opening to clamp the conduit in place) can provide for efficient routing and the ability to quickly and effectively route the conduit during manufacturing.

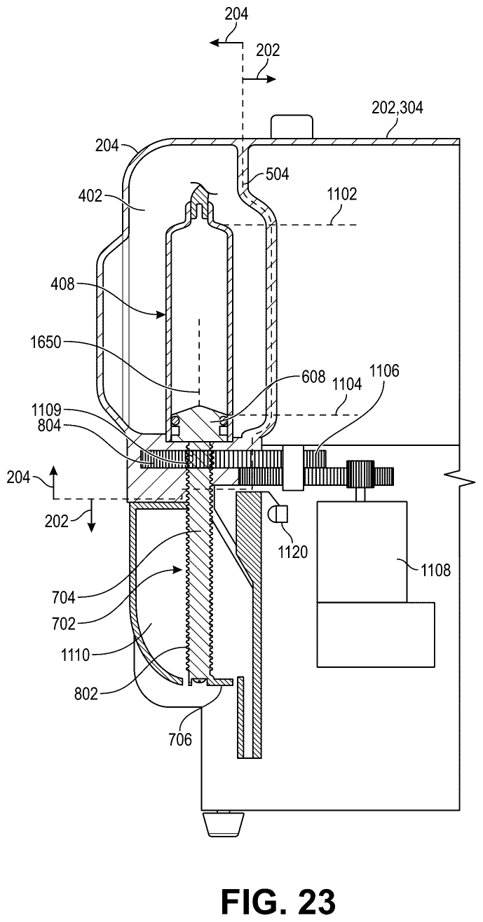

Referring to FIG. 23, a cross-sectional view of a fluid transfer cartridge mounted in a cartridge receptacle of a generator of a treatment system is shown in accordance with an embodiment. In cross-section, the interaction between the fluid transfer cartridge 204 and generator 202 can be recognized. For example, the back plate 504 of the fluid transfer cartridge 204 is seen to appose and conform to the generator 202 when the fluid transfer cartridge 204 is received within the cartridge receptacle 302. The separation between the fluid transfer cartridge 204 and the generator 202 is depicted in FIG. 23 by a dotted line. When received, the fluid transfer cartridge 204 can be actuated to drive the syringe shafts 704 from a home position 1102 to an end position 1104. More particularly, a generator gear 1106 (or gear train), which is driven by a motor 1108 of the generator 202, can mesh with and drive the gear 804 of the fluid transfer cartridge 204 to concurrently move the syringe piston 702 along the syringe axis 1650. For example, the motor 1108 can be operably coupled to the shaft 704, and actuated by the one or more processors of the control unit, to cause an internal thread 1109 of the gear 804 to move the external thread 802 of the shaft 704 (and the syringe piston 702) during fluid transfer by the syringe.

When the stopper 608 is at the end position 1104, the shaft end 706 is outside of the cartridge cavity 402. In an embodiment, the generator housing 304 includes a well 1110 below the cartridge receptacle 302. The well 1110 can have an outer wall surrounding a space within which the shaft end 706 resides when the stopper 608 is at the end position 1104. The well 1110 can therefore receive the syringe piston 702 of the fluid transfer cartridge 204 during fluid transfer by the syringe. When the syringe piston 702 is contained within the well 1110, the shaft 704 can be protected from damage. More particularly, the well 1110 can shroud the shaft 704 such that it does not contact an external surface that might strain the syringe components. Similarly, the well 1110 shields the shaft 704 from the user, thereby reducing pinch points that could otherwise injure the user. Finally, by containing the shafts 704 within the generator 202, rather than extending the shafts 704 outward from the control unit, an overall form factor of the control unit may be compacted.

The well 1110 may be dimensioned to accommodate several functions. First, a size of the space within the well 1110 can allow the user to easily clean out the well 1110, e.g., by wiping the internal surfaces of the well 1110. Furthermore, a height of the well 1110 may be greater than a length of the syringe throw to ensure that the shaft end 706 does not directly contact the internal surface of the well 1110 when the stopper 608 reaches the end position 1104. Additionally, a depth of the well 1110 can allow the fluid transfer cartridge 204 to be removed from the generator 202 without requiring the stopper 608 to be homed. More particularly, when removing the cartridge with the shaft 704 fully extended, the cartridge may be tilted forward and there may be enough space within the well 1110 to allow the shaft 704 to be tilted and removed upward from the well cavity.

As described above, the syringe can be operated to move the stopper 608 within the syringe barrel 408 between the home position 1102 and the end position 1104. The stopper 608 may be placed at the home position 1102, for example, during shipment to the procedure room. The home position 1102 may also be a location at which, after the stopper 608 has been cycled one or more times between the home position 1102 and the end position 1104, the syringe barrel 408 is purged of air bubbles. More particularly, the travel of the syringe can include the home position 1102 of the syringe piston 702 in which the syringe piston 702 removes bubbles from the syringe. Similarly, when the stopper 608 is at the end position 1104, the cooling fluid 603 may be drawn into the syringe barrel 408 to fill the syringe. When the stopper 608 is moved to the end position 1104 after purging the syringe barrel 408, the cooling fluid 603 within the syringe may be free of bubbles. The method of purging the syringes is described in further detail below, however, at this stage it will be appreciated that movement of the syringe piston 702 is effected by the motor 1108, which may be controlled by one or more processors of the control unit.

The movement of the syringe piston 702 can be controlled by the one or more processors through control of the motor 1108. For example, the motor 1108 may be a stepper motor 1108, and the processors can drive the stepper motor 1108 through a predetermined angular rotation that, when considering a gearing ratio between the generator gear 1106 (or gear train) and the gear 804 of the cartridge, will result in a predetermined axial movement of the shaft 704. Additionally, the control unit may incorporate sensors to detect a position of the syringe piston 702, e.g., the shaft end 706. By sensing the shaft position, the one or more processors can determine a location of the stopper 608 within the syringe barrel 408, and thus, an amount of fluid contained within the syringe.

The sensors used to detect the shaft position may be magnetic, optical, mechanical, etc. In an embodiment, the sensors include one or more position switches 1120. For example, the treatment system 100 may include a position switch 1120, e.g., an optical switch such as an optical sensor, configured to detect the syringe piston 702 when the stopper 608 is at the home position 1102. An optical sensor may provide better resolution than a magnetic switch. A magnetic sensor, on the other hand, may require less maintenance than an optical sensor. Accordingly, the position switch 1120 can be selected based on design needs.

Optionally, a second position switch 1120 (not shown) may be configured to detect the syringe piston 702 when the stopper 608 is at the end position 1104. The position switch(es) 1120 may be mounted within or along a wall of the generator well 1110. For example, the position switch(es) 1120 can be mounted along a rear wall of the well 1110. When more than one switches are incorporated, the switches can be aligned in a series along a vertical axis that parallels and runs adjacent to the syringe axis 1650.

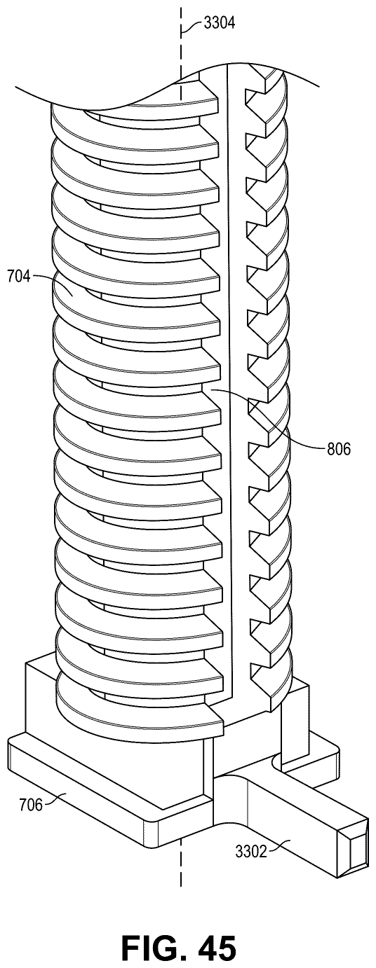

In an embodiment, the shaft end 706 includes an element that can trigger the position switch(es) 1120. For example, the element can include an optical tab to trigger an optical sensor. The optical tab is described further with respect to FIG. 45, below, and may be mounted at the shaft end 706. The optical tab can travel along the vertical axis and pass by the optical switch(es) as the syringe piston travels vertically along the syringe axis 1650. The optical switch(es) of the generator 202 can interact with the optical tab mounted on the syringe piston 702. For example, the optical tab may be disposed on the shaft 704 at the shaft end 706. When the optical tab is in proximity to the optical switch, e.g., adjacent to the optical switch, the optical tab can block the optical switch. For example, the optical sensor can have a light emitting diode to emit a light signal, and the tab can obstruct or reflect the light signal of the optical sensor. Accordingly, when the state of the optical switch changes, it indicates proximity between the optical tab and the optical switch. Such proximity between the optical tab and the optical switch can be detected and used by the one or more processors of the control unit to determine travel of the one or more syringes of the fluid transfer cartridge 204. More particularly, the one or more processors can monitor the state of the optical switch(es) to identify where the syringe pistons 702 are located relative to syringe barrels 408, and more particularly, a position of the shaft 704 and/or whether the stopper 608 is at the home position 1102, the end position 1104, or located at an intermediate position between the start and end of travel.

In an embodiment, the shaft end 706 includes an element that can trigger the position switch 1120. For example, the element can include a magnet, which may be mounted at the shaft end 706, and can travel along the vertical axis and pass by the switch(es) as the syringe piston 702 travels vertically along the syringe axis 1650. The magnetic switch(es) of the generator 202 can interact with the magnet mounted on the syringe piston 702. For example, the magnet may be disposed on the shaft 704 at the shaft end 706. When the magnet is in proximity to the magnetic switch, e.g., adjacent to the magnetic switch, the magnet moves contacts of the magnetic switch. Accordingly, when the state of the magnetic switch changes, it indicates proximity between the magnet and the magnetic switch. Such proximity between the magnet and the magnetic switch can be detected and used by the one or more processors of the control unit to determine travel of the one or more syringes of the fluid transfer cartridge 204. More particularly, the one or more processors can monitor the state of the magnetic switches to identify where the syringe pistons 702 are located relative to syringe barrels 408, and more particularly, whether the stopper 608 is at the home position 1102, the end position 1104, or located at an intermediate position between the start and end of travel.

The position switches 1120 may act as limit switches to provide information to the control unit that may be used to prepare the syringes for fluid delivery. Given that the magnetic switches do not require precise alignment between the magnet and the magnetic switch, magnetic switches may provide more reliable limit switches than, for example, mechanically-activated switches. Accordingly, one or more processors of the control unit can use the switch signals from the magnetic switches to control a prep cycle of the syringes.

In the prep cycle, the control unit cycles the syringes, using the limit switches to detect whether the syringes are appropriately filled with the cooling fluid 603, whether the fluid conduit 206 and the manifold of the fluid transfer cartridge 204 are filled with the cooling fluid 603, and to ensure that air bubbles are removed from the fluid conduit 206, the manifold, and the syringes. The control unit purges the syringes during the prep cycle to achieve these goals.

The fluid transfer cartridge 204 can be installed on the generator 202 with the stopper 608 in the home position 1102. When in the home position 1102, the shaft end 706 can be within the cartridge cavity 402, and thus, above and outside of the well 1110. In the home position 1102, the syringe is empty. To begin the prep cycle, the one or more processors of the control unit can drive the motor 1108 to actuate the syringe piston 702 in the downward direction. Movement of the stopper 608 generates a vacuum within the syringe barrel 408 and draws cooling fluid 603 into the syringe. Accordingly, an initial stage of the prep cycle fills the syringe with the cooling fluid 603.

As the syringe fills with fluid, the shaft end 706 moves into the well 1110 outside of the cartridge cavity 402. The one or more processors can determine a level or amount of fill of the syringes. For example, when the stopper 608 is at the end position 1104 in the syringe barrel 408, the shaft end 706 can be disposed adjacent to a lowest position switch 1120 of the generator 202. The proximity of the shaft end to the position switch can trigger, e.g., close (or open) the contacts of the switch, to generate a switch signal that is sent to the one or more processors. The processors can determine, based on the switch signal, that the syringes are filled with a predetermined amount of cooling fluid 603. For example, the predetermined amount of cooling fluid 603 may be a volume of the syringe barrel 408.

While the predetermined amount of cooling fluid 603 may correspond to a determination that the stopper 608 is in the end position 1104, the one or more processors may be configured to determine other fill levels. For example, one or more intermediate position switches 1120 may be placed between an uppermost position switch corresponding to the home position 1102 and the lowermost position switch corresponding to the end position 1104. The placement of the position switches may be selected to correspond to known volumes of fluid within the syringe. For example, position switches may be placed at locations corresponding to 10 mL increments of syringe fill levels.

When the syringes are filled, the prep cycle can proceed. The one or more processors may drive the motor 1108 to cause the syringe piston 702 to move upward to expel cooling fluid 603 from the syringes. The cooling fluid 603 is expelled into a fluid path of the cartridge. For example, the cooling fluid 603 can flow out of the syringe into the fluid conduit 206 that connects the fluid transfer cartridge 204 to the catheter 101. In an embodiment, the syringes are entirely emptied of the cooling fluid 603. The one or more processors may determine that the one or more syringes are empty based on switch signals generated by the position switches. The switch signals can indicate that the stopper 608 is in the home position 1102, and thus, the syringes are empty.

Driving the cooling fluid 603 out of the syringe can remove bubbles from one or more of the syringes or the fluid path of the fluid transfer cartridge 204. More particularly, air that is drawn into the syringe when the stopper 608 is initially moved from the home position 1102 to the end position 1104 can be expelled from the fluid network when the stopper 608 is then driven back toward the home position 1102. Purging the fluid network of air can prime the system to ensure that the fluid network is filled with incompressible fluid, e.g., sterile water, and stable.

In an operation of the prep cycle, after purging air from the syringe, the syringe may be filled with the cooling fluid 603. The one or more processors can drive the motor 1108 to move the stopper 608 from the home position 1102 to the end position 1104. Accordingly, the cooling fluid 603 can be drawn into the syringe barrel 408. After the purging operation, the fluid drawn into the syringe barrel 408 can be mostly or entirely bubble free. In an embodiment, the purging operation may be repeated one or more times until the cooling fluid 603 within the syringe is entirely bubble free.

It will be appreciated that the magnetic switches provide one type of limit switch, and other types of limit switches may be incorporated into the treatment system 100. In an embodiment, the limit switches are optical switches, e.g., optical proximity sensors 2910. Accordingly, the shaft end 706 may be configured to emit or reflect light to a light sensor of the treatment system 100, e.g., a light sensor located within the generator 202.

The optical limit switch may include a light source mounted on the shaft end 706. The light source can shine radially outward from a shaft axis 1650, e.g., toward the rear wall of the well 1110 of the generator 202. The rear wall may include one or more light sensors to receive the light that is emitted from the shaft end 706. Alternatively, the rear wall may include proximity light sensors that emit light toward the shaft end 706, and receive the reflected light that is returned from the shaft end 706. Accordingly, the light sensor can detect when the shaft end 706 is proximate to the light sensor, based on the detected light, and send a corresponding switch signal to the one or more processors of the treatment system 100. The processor(s) may use the switch signals to detect and control shaft position, as described above. Accordingly, magnetic switches are a non-limiting example of a limit switch that may be integrated in the treatment system 100, and optical or mechanical switches may also be used to detect shaft position.

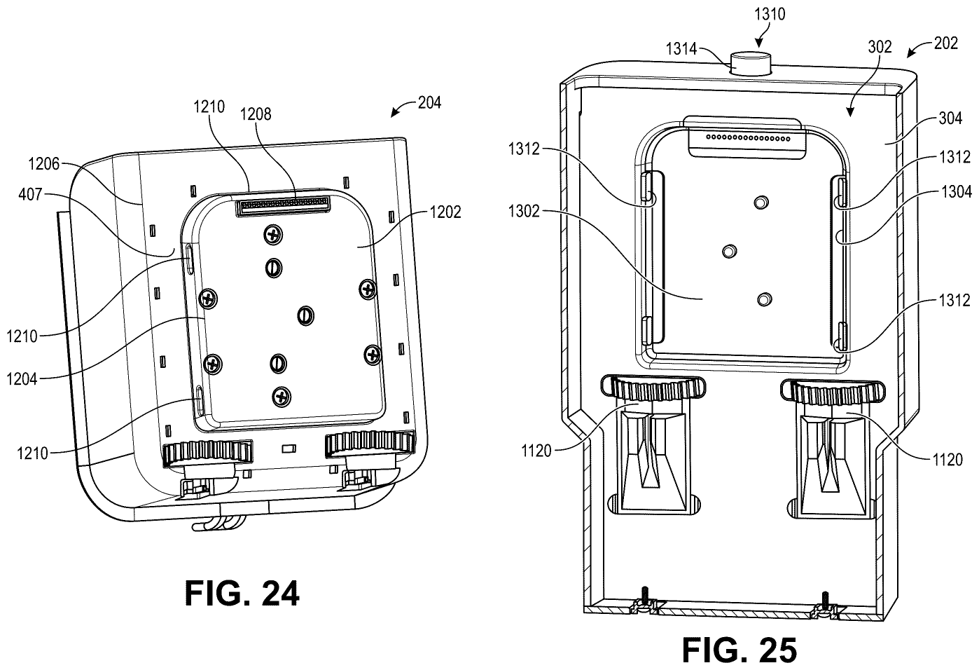

Referring to FIG. 24, a rear perspective view of a fluid transfer cartridge is shown in accordance with an embodiment. The rear face 407 of the fluid transfer cartridge 204 may appose a surface of the generator 202 defining the cartridge receptacle 302 when the cartridge is mounted on the generator 202. The rear face 407 can include a boss 1202 projecting outward from a surrounding base surface. In an embodiment, the boss 1202 has a boss perimeter 1204 extending around the portion of the rear face 407 on the boss 1202. The boss perimeter 1204 may be laterally inward of an outer perimeter of the rear face 407. More particularly, a rear face boundary 1206 can define an edge separating the rear face 407 from the top, side, and bottom surfaces of the cartridge. Accordingly, the rear face 407 can have a portion covering the base surface laterally outward of the portion covering boss 1202. The rear face portions may be offset from each other, e.g., at different locations in the rearward direction. Accordingly, the rear surface has a stepped profile when viewed from the side.

In an embodiment, the fluid transfer cartridge 204 includes one or more electrical contact pads 1208 or pins to connect to corresponding circuitry of the generator 202. More particularly, the electrical contact pads 1208 or pins can connect to an electrical circuit board. For example, the electrical circuit board can be a pressure sensor board having sensors and/or processors configured to detect and/or determine pressure along fluid pathways of the control unit. The electrical contact pads 1208 or pins may include spring-loaded electrical contact pins that are exposed through the rear face 407 of the cartridge near an upper end of the boss 1202. The electrical contact pads 1208 may include conductive contact pads exposed through the rear face 407 near the upper end of the boss 1202 and located to make contact with spring-loaded electrical contact pins extending from the generator 202. In this case, the upper end may refer to a side of the boss perimeter 1204 farthest from the floor. The electrical contact pads or pins and their placement at the upper region of the cartridge can provide several benefits. First, in the case of the contact pins, the spring-loaded structure permits the pins to deflect when placed in contact with the generator 202. Thus, the pins can conform to the generator 202 in the event of misalignment or movement between the components during operation. Such benefit is similarly realized when the contact pads of the fluid transfer cartridge 204 engage contact pins of the generator 202. Accordingly, the deflectable pins allow a better connection between the generator 202 and the cartridge during operation. Second, placement of the pads or pins along the top of the cartridge can reduce a likelihood of electrical shorting in the event of a conduit leak. More particularly, the pads or pins can be placed vertically above the fluid path that is internal to the cartridge shell 306 such that any leak from the fluid path will fall downward to the floor without contacting (and potentially shorting) the electrical connections.

Referring to FIG. 25, a front perspective view of a cartridge receiving portion of a generator is shown in accordance with an embodiment. The boss 1202 of the fluid transfer cartridge 204 may engage a corresponding feature in the generator 202. In an embodiment, the cartridge receptacle 302 includes a back recess 1302 to receive the boss 1202. The back recess 1302 can have a recess perimeter 1304 extending around the surface of the generator 202 that is recessed. The recess perimeter 1304 of the back recess 1302 can have a profile that matches a profile of the boss perimeter 1204. For example, the boss 1202 may be a rectangular protrusion and the back recess 1302 may be a rectangular depression. The boss 1202 may therefore engage and fill the back recess 1302 such that a lateral side wall of the boss 1202 apposes and conforms to a lateral side wall of the back recess 1302. Similarly, the rear surface of the fluid transfer cartridge 204 can appose and conform to a front surface of the generator housing 304. The conforming surfaces of the components can stabilize the fluid transfer cartridge 204 relative to the generator 202 and minimize movement between the components during operation.

The contact pads or pins of the fluid transfer cartridge 204 can extend toward or face a slot located in an upper region of the back recess 1302. The slot can expose one or more electrical contacts that the contacts of the fluid transfer cartridge 204 can engage when the cartridge is mounted on the generator 202. Accordingly, an electrical connection can be made between the cartridge and the generator 202 to communicate signals, including switch signals, light activation signals, etc.

To further stabilize the control unit components and secure the fluid transfer cartridge 204 to the generator 202, the treatment system 100 can include a fastening mechanism 1310 to latch the fluid transfer cartridge 204 within the cartridge receptacle 302. The fastening mechanism 1310 can include corresponding catches 1312 and recesses arranged on the fluid transfer cartridge 204 and the generator 202. Referring again to FIG. 24, the boss 1202 may include several recesses 1210 distributed around the boss perimeter 1204 of the boss 1202. By way of example, the boss 1202 can have a rectangular profile and four or more recesses 1210 may be located within the lateral side wall of the boss 1202 around the boss perimeter 1204. In an embodiment, each of the four recesses 1210 can be located adjacent to a respective corner of the rectangular profile. Distributing the recesses 1210 around the boss perimeter 1204 can distribute the retention load applied to the fluid transfer cartridge 204 by the generator 202, and thus, may optimally stabilize the cartridge relative to the generator housing 304.

Referring again to FIG. 25, the fastening mechanism 1310 can include several catches 1312 distributed around the recess perimeter 1304. By way of example, the back recess 1302 can have a rectangular profile and four latches may be located along the lateral side wall of the back recess 1302 around the recess perimeter 1304. In an embodiment, there are four latches at locations corresponding to the locations of recesses 1210 in the boss 1202. More particularly, the catches 1312 may be configured to engage the recesses 1210 of the boss 1202 to secure the fluid transfer cartridge 204 to the generator 202.

In an embodiment, each catch 1312 is a spring-loaded catch 1312 operably coupled to a release button 1314 of the fastening mechanism 1310. The release button 1314 may be movable between a latched position and an unlatched position. For example, the release button 1314 may be in a latched position when it is fully extended (not pressed). Pressing the release button 1314 can move the button from the latched position to an unlatched position. The release button 1314 can be operably coupled to the catches 1312 such that moving the release button 1314 from the latched position to the unlatched position causes the catches 1312 to move out of the cartridge receptacle 302. More particularly, movement of the release button 1314 can cause the catches 1312 to move from an extended position within the recesses 1210 of the cartridge to a recessed position outside of the boss recesses 1210. When the catches 1312 are engaged with the boss recesses 1210, the cartridge is secured to the generator 202. By contrast, when the catches 1312 retract from the boss recesses 1210, the cartridge may be released from the generator 202. Accordingly, the fastening mechanism 1310 provides a quick release mechanism to install and remove the fluid transfer cartridge 204 from the generator 202.

In addition to providing a quick release mechanism, the fastening mechanism 1310 promotes a secure and stable mechanical connection between the cartridge 204 and the generator 202. The distributed latches around the boss 1202 and back recess 1302 ensure that mechanical loading of the cartridge during operation is evenly distributed, and thus, the latches can share the loading and minimize deflection at any given location around the cartridge. Although the fastening mechanism 1310 can have four or more friction points at which the catches 1312 engage the recesses 1210, the cartridge can be disengaged smoothly using the release button 1314 that actuates the spring-loaded latches. In an embodiment, the catches 1312 are driven by a linkage system, a plate having a cam mechanism, or another intermediate structure between the release button 1314 and the catch 1312. Such mechanisms can operate smoothly and in a manner that provides a favorable degree of tactile feedback to the user. The fastening mechanism 1310 may therefore advantageously secure the cartridge to the generator 202 in a user-friendly manner.

The fastening mechanism 1310 may include one or more catches 1312 that are operably coupled to the release button 1314 such that moving the release button 1314 from the latched position to the unlatched position causes the one or more catches 1312 to move out of the cartridge receptacle 302. The catches 1312 need not be directly spring-loaded, but rather, may be biased in relation to the release button 1314. More particularly, the release button 1314 can be movable from the latched position to the unlatched position, and the release button 1314 may be operably coupled to one or more springs to bias the release button 1314 toward the latched position. Accordingly, the one or more springs can bias the catch(es) 1312 into the cartridge receptacle 302.

In an embodiment, the one or more springs that bias the release button 1314 is a single spring. More particularly, the release button 1314 can be driven to the latched position by the single spring. The one or more catches 1312, by contrast, can include several catches 1312 that are interconnected by a linkage. More particularly, the linkage can interconnect the catches 1312 such that movement of the release button 1314 acts as an input to cause linkage movement that in turn drives the catches 1312 into and out of the cartridge receptacle 302. When the release button 1314 is moved from the latched position to the unlatched position, the one or more catches 1312 can move out of the cartridge receptacle 302.

The treatment system 100 can include one or more processors configured to execute instructions stored in a non-transitory computer-readable medium to cause the treatment system 100 to perform various methods, such as the prep cycle described above. The methods can include methods of providing visual feedback to a user to indicate that electrical connections have been made between components of the treatment system 100 or between the treatment system and external components. Several such methods are described below.

In an embodiment, the treatment system 100 can illuminate the syringe upon mounting the fluid transfer cartridge 204 on the generator 202. In an operation, the one or more processors are configured to determine whether the fluid transfer cartridge 204 is received within the cartridge receptacle 302. Detection of cartridge mounting may be made by various sensors. For example, one or more of the electrical contact pads 1208 may engage a corresponding electrical contact of the generator 202 when the cartridge is received in the cartridge receptacle 302. The electrical contact can cause an input signal to be sent to the one or more processors. In response to detecting the input signal, and thus, in response to determining that the fluid transfer cartridge 204 is received within the cartridge receptacle 302, one or more processors can activate the light source 1850 of the fluid transfer cartridge 204. The light source 1850 may be directed toward the syringe, as described above. Accordingly, when the syringe becomes end-lit, the user is provided with visual feedback to confirm that the components of the treatment system 100 are engaged and ready for operation.

In an embodiment, the treatment system 100 includes one or more indicator lights 452 to indicate that a connection between the generator 202 and one or more external components has been made. Referring again to FIG. 16, the generator 202 may include one or more electrical connectors 450 configured to connect to external connectors of corresponding external components. For example, an electrical connector 1652 of the generator 202 may be an electrical socket to receive the external connector 1352 of the catheter 101. Additional electrical connectors 450 may include plugs to receive external connectors of other components, such as a remote control device. The external connectors can be plugs that engage the sockets of the external connectors, or vice versa.

The generator 202 can include an indicator light 452 located near the electrical connector 1652. For example, the indicator light 452 can be a single light-emitting diode (LED) adjacent to the electrical socket, or several LEDs positioned around the socket. In an embodiment, the indicator light 452 includes an indicator light ring 454 extending around the electrical connector 1652. More particularly, as shown in FIG. 16, the indicator light ring 454 can include an annular bezel that circumferentially surrounds the electrical connector 1652. One or more LEDs can be mounted behind the bezel such that illumination of the lights causes the bezel to appear as a solid light ring. The light ring can allow the light to be viewed from any direction without being blocked from view, e.g., by a cable or catheter 101.

The indicator light 452 can have a lighting state or mode that provides visual feedback to the user. For example, the one or more processors of the treatment system 100 may be configured to determine whether the electrical connector 1652 is electrically connected to the external connector 1352. By way of example, when the external connector 1352 is plugged into the electrical connector 1652, a signal may be sent to the one or more processors that indicates, and allows the processors to determine, that the connection has been made. In an operation, in response to determining that the electrical connector 1652 is connected to the external connector 1352, a lighting mode of the indicator light 452 can change. In an embodiment, the indicator light 452 changes from a deactivated, unlit state to an activated, lit state. Accordingly, the user can see that the indicator light 452 has become lit to confirm that the external component is electrically connected to the generator 202.

The change in lighting mode from an unlit to a lit state is provided as a non-limiting example. Alternatively, the change in the lighting mode may be from a first lighting mode in which the indicator light 452 emits light, e.g., blinks, to a second lighting mode in which the indicator light 452 continuously emits lights, e.g., the lighting is viewed as being solid. In another alternative, the change in the lighting mode may be from a first lighting mode in which the indicator light 452 emits a first color of light, e.g., red, to a second lighting mode in which the indicator light 452 emits a second color of light, e.g., blue. In any case, the change in the lighting mode provides visual feedback to the user that the external component is electrically connected to the generator 202 and therefore ready for use.

Referring to FIG. 26, an exploded view of a cartridge shell of a fluid transfer cartridge is shown in accordance with an embodiment. As described above, the cartridge shell 306 of the fluid transfer cartridge 204 can include the handle front plate 1750 and the back plate 504. When combined, the handle front plate 1750 and the back plate 504 can define the cartridge cavity 402 centrally located between the various walls and faces.

When snapped or otherwise fit together, the handle front plate 1750 and the back plate 504 can contain, within the cartridge cavity 402, one or more components to provide fluid transfer functionality. For example, the fluid transfer cartridge 204 can include the syringe holder 513 to hold the syringe barrels 408, 412 within the cartridge cavity 402. The syringe holder 513 can stabilize the syringes during fluid delivery. The fluid transfer cartridge 204 can also include tubing 1406 to facilitate the movement of fluid from the syringes to the catheter 101.

The use of tubing to transfer fluid throughout the fluid transfer cartridge 204 may require long conduit lines and many glue joints to achieve the fluid pathway and interconnections that are needed for fluid transfer. For example, the exclusive use of tubing could require more than five feet of tubing and forty glue joints to create the fluid network. Such a fluid network, however, could occupy a substantial volume, could lead to leaks and/or flow inconsistency at the glued joints, and may be challenging to assemble during manufacturing. In an embodiment, a cartridge manifold 1402 may be used to replace much of the tubing length and joints, thereby providing a more compact, reliable, and easier to manufacture fluid network. Due to the reduced size and weight of the fluid network, the corresponding size and weight of the fluid transfer cartridge 204 may also be reduced, allowing more cartridges to be sterilized at once and more cartridges to be shipped per unit volume.

The cartridge manifold 1402 may replace some, but not all, of the fluid tubing within the fluid transfer cartridge 204. By way of example, the syringe barrel 408 may have a syringe cavity 1404, which is connected to fluid channels of the cartridge manifold 1402 by one or more conduits 1406. Other conduits 1406, e.g., between the cartridge manifold 1402 and the second syringe barrel 412, the balloon catheter 101, a fluid reservoir, etc., may also be routed through the cartridge cavity 402. Such conduits 1406 are not shown in FIG. 26 to avoid cluttering the illustration.

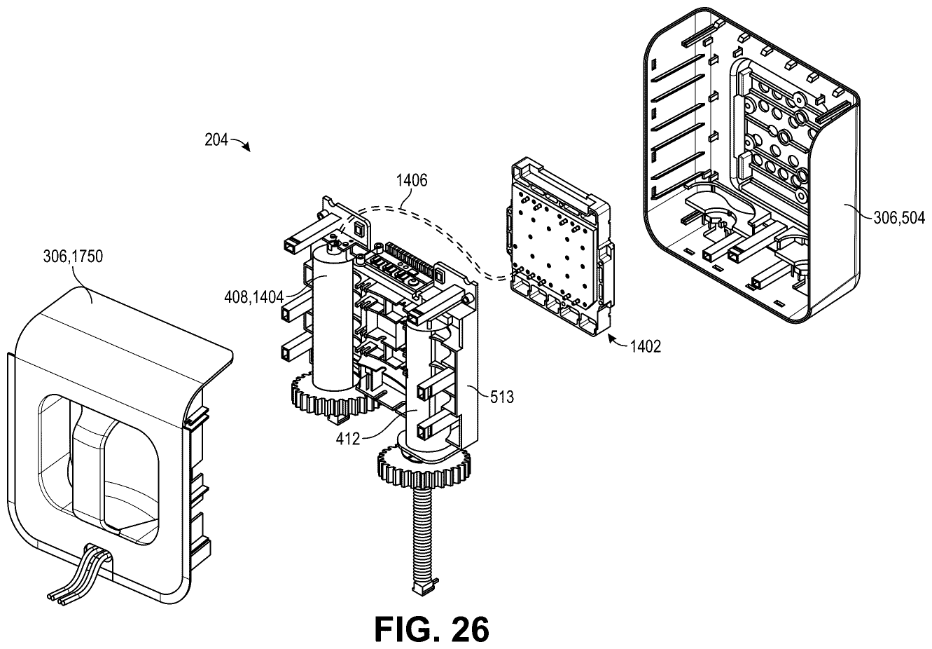

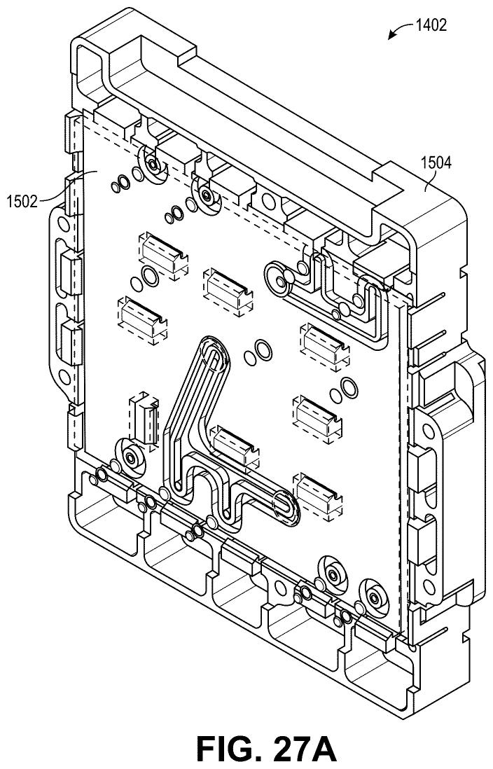

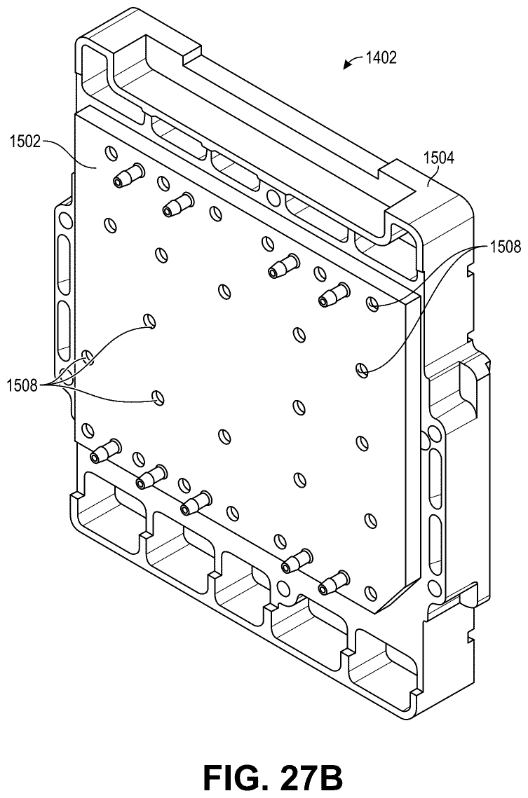

Referring to FIG. 27A, a perspective view of a cartridge manifold is shown in accordance with an embodiment. The cartridge manifold 1402 can include several plates assembled to each other. In an embodiment, the cartridge manifold 1402 includes a fore plate 1502 assembled to an aft plate 1504. The fore plate 1502 and the aft plate 1504 can be secured to each other. For example, the fore plate 1502 may be snap fit, e.g., secured by snap closures, to the aft plate 1504. As described below, the fore plate 1502 and the aft plate 1504 can secure an intermediate plate that has channels and ports to move cooling fluid throughout the cartridge manifold 1402, and to exchange the cooling fluid with external components such as the balloon catheter 101 and the fluid reservoir. The fore plate 1502 is rendered transparent to allow the intermediate plate to be viewed in FIG. 27A.

Referring to FIG. 27B, a perspective view of a cartridge manifold is shown in accordance with an embodiment. Alternatively, the fore plate 1502 and the aft plate 1504 may be fastened by screws or otherwise secured to each other. More particularly, several manifold fasteners 1508 can extend through through-holes in the aft plate 1504 and screw into threaded holes formed in the fore plate 1502. The fasteners 1508 can hold the plates together to sandwich an intermediate plate, as described below.

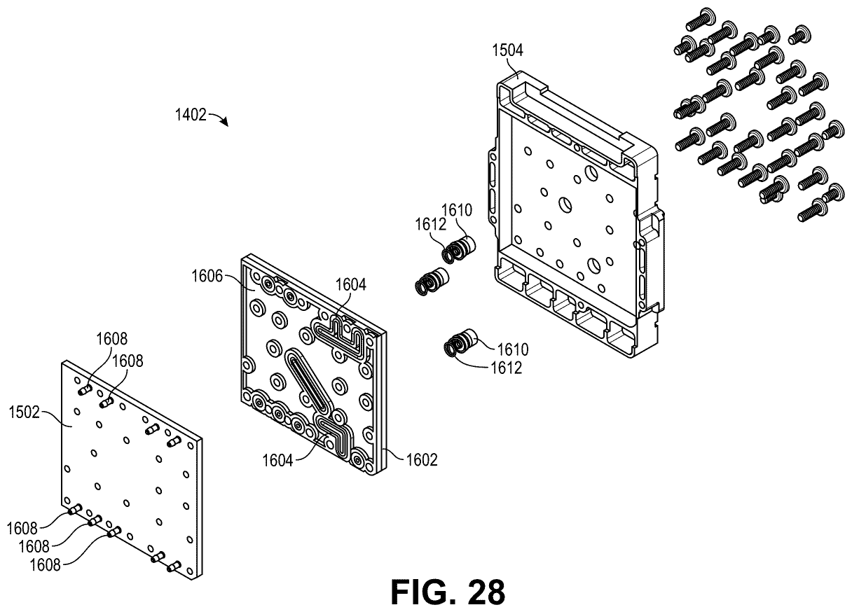

Referring to FIG. 28, an exploded view of a cartridge manifold is shown in accordance with an embodiment. The cartridge manifold 1402 in the cartridge cavity 402 can include a fluid transfer plate 1602 sandwiched between the fore plate 1502 and the aft plate 1504. The fluid transfer plate 1602 can include channels on front and rear surfaces that are connected through various ports in the plate. More particularly, one or more front fluid channel 1604 in a front plate surface 1606 can carry the cooling fluid, via channels on the rear surface, to one or more outlet ports 1608 for transfer to the external components.

In an embodiment, the outlet ports 1608 of the fore plate 1502 connect to external components. More particularly, the outlet ports 1608 can include fittings, e.g., barb fittings, which connect to fluid conduits 1406, and those conduits can extend to connect to external components, such as syringes, fluid reservoirs, the balloon catheter 101, or pressure sensors. Accordingly, the fore plate outlet ports 1608 can function as fluid interfaces to the external components. Through the outlet ports 1608, fluid can be transferred into and out of the cartridge manifold 1402. In an embodiment, the fore plate 1502 includes four outlet ports 1608 along an upper edge and five outlet ports 1608 along a lower edge, although the number and locations of these outlet ports 1608 can be varied according to a layout of the external components and the fluid transfer cartridge 204.

Movement of the fluid through the channels and ports of the cartridge manifold 1402 can be controlled by one or more pistons 1610. Each piston 1610 may be associated with, or include, a spring 1612. More particularly, the piston 1610 may be spring-loaded to bias the piston 1610 to a given position. For example, as described below, the spring 1612 may bias the piston 1610 to an open position and a solenoid can actuate the piston 1610 to move the piston 1610 to a closed position. In particular, the piston 1610 can be moved between positions that seal or unseal fluid ports in the fluid transfer plate 1602 to start or stop flow of the cooling fluid 603 through the fluid channels.

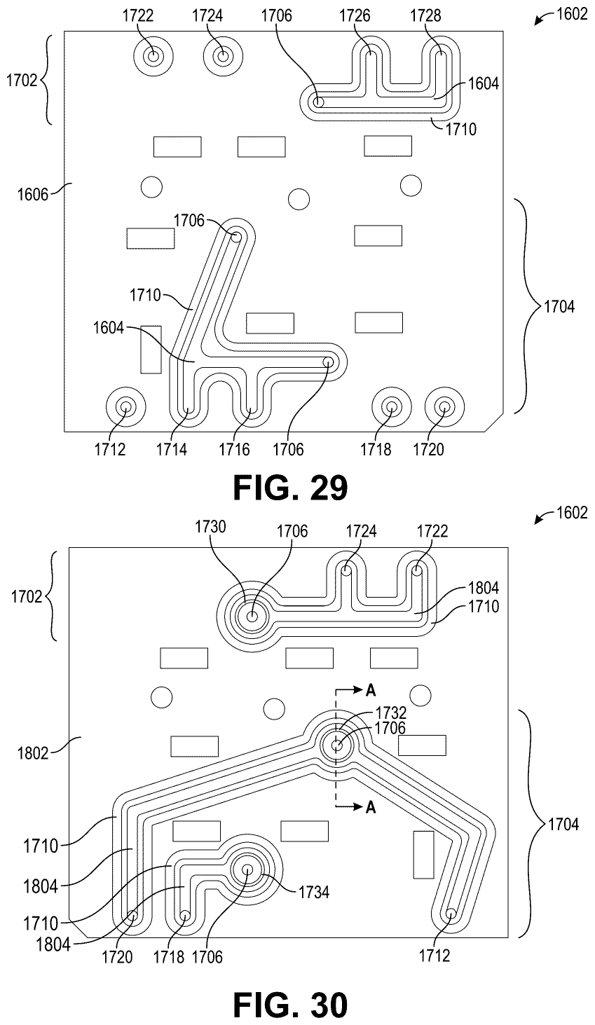

Referring to FIG. 29, a front view of a fluid transfer plate of a cartridge manifold is shown in accordance with an embodiment. The front plate surface 1606 of the fluid transfer plate 1602 can include several front fluid channels 1604. In an embodiment, the front fluid channels 1604 belong to respective fluid circuits. More particularly, some channels and ports may belong to an upper fluid circuit 1702, and other channels and ports may belong to a lower fluid circuit 1704. Each of the fluid circuits can include respective front fluid channels 1604 and one or more outlets. The fluid channels and outlets, as described below, can be interconnected with outlet ports 1608 of the fore plate 1502 to transfer fluid to and from external components. Furthermore, the fluid transfer plate 1602 can include one or more fluid port 1706. Each fluid port 1706 can extend through the fluid transfer plate 1602 from the front fluid channels 1604 in the front plate surface 1606 to rear fluid channels in a rear plate surface (FIG. 30). Accordingly, cooling fluid 603 can be moved from the channels in front of the fluid transfer plate 1602 to channels behind the fluid transfer plate 1602 through the fluid ports 1706.

In an embodiment, the fluid channels of the fluid transfer plate 1602 may be surrounded by respective channel seals 1710. The channel seals 1710 can be gaskets, e.g., O-rings, or strips of elastomeric material having circular, rectangular, cross-shaped, or other cross-sectional profiles, which are placed along an outer perimeter of the fluid channels. The seals can be fit into grooves, overmolded into the plate, or otherwise attached to the fluid transfer plate 1602. When the fluid transfer cartridge 204 is assembled, the channel seals 1710 can be sandwiched between the fluid transfer plate 1602 and an adjacent fore plate 1502 or aft plate 1504. The sandwiched seals can form a hermetic seal around the fluid channels to isolate the cooling fluid 603 within the channels.

The lower fluid circuit 1704 may be associated with the syringe barrel 408 that is used to deliver fluid to the balloon catheter 101. More particularly, cooling fluid 603 may be transferred from an external fluid reservoir, e.g., a fluid-filled bag, through the lower fluid circuit 1704 to the syringe barrel 408. The outlets of the front plate surface 1606, which connect to respective outlet ports 1608 of the fore plate 1502, can be labeled for ease of reference. For example, the lower fluid circuit 1704 can have an L1 outlet 1712, an L2 outlet 1714, an L3 outlet 1716, an L4 outlet 1718, and an L5 outlet 1720. Each of the L1-L5 outlets 1720 can connect to fittings on the fore plate 1502 that are in turn connected to conduits 1406. More particularly, the L1-L5 outlets can be in fluid communication with the outlet ports 1608 along the lower edge of the fore plate 1502. Those conduits 1406 may connect to external components such as the fluid reservoir, the syringe barrel 408, an inlet line of the balloon catheter 101, and/or one or more pressure sensors.

The upper fluid circuit 1702 may be associated with the second syringe barrel 412 that is used to draw fluid from the balloon catheter 101. More particularly, cooling fluid 603 may be transferred from the balloon catheter 101 through the upper fluid circuit 1702 to transfer the fluid to the external fluid reservoir. The outlets of the front plate surface 1606, which interconnect to respective outlet ports 1608 of the fore plate 1502, can be labeled for ease of reference. For example, the upper fluid circuit 1702 can have a U1 outlet 1722, a U2 outlet 1724, a U3 outlet 1726, and a U4 outlet 1728. Each of the U1-U4 outlets can connect to fittings on the fore plate 1502 that are in turn connected to conduits 1406. More particularly, the U1-U4 outlets can be in fluid communication with the outlet ports 1608 along the upper edge of the fore plate 1502. Those conduits 1406 may connect to external components such as an outlet line of the balloon catheter 101, the second syringe barrel 412, the fluid reservoir, and/or one or more pressure sensors.

It is apparent that some of the outlets are in fluid communication with each other through the fluid channels. For example, the U3 outlet 1726 and the U4 outlet 1728 are in fluid communication with each other through the front fluid channel 1604 of the upper fluid circuit 1702. Similarly, the L2 outlet 1714 and the L3 outlet 1716 are in fluid communication with each other through the front fluid channel 1604 of the lower fluid circuit 1704. As described below, the outlets that are isolated on the front side of the fluid transfer plate 1602, e.g., the U1, U2, L1, L4, and L5 outlets 1720, may also be in fluid communication with other outlets through fluid channels on the back side of the fluid transfer plate 1602. More particularly, each outlet and/or channel can include a respective fluid port 1706 extending through the fluid transfer plate 1602 to connect to corresponding channel(s) on the back side of the fluid transfer plate 1602.

Referring to FIG. 30, a rear view of a fluid transfer plate of a cartridge manifold is shown in accordance with an embodiment. The cartridge manifold 1402 includes a rear plate surface 1802 having one or more rear fluid channels 1804. Like the front fluid channels 1604, the rear fluid channels 1804 can be surrounded by channel seals 1710 to isolate fluid within the fluid channels. For example, the aft plate 1504 can be apposed to the rear plate surface 1802 such that the channel seals 1710 are sandwiched between the rear plate surface 1802 and the aft plate 1504. By extending around the rear fluid channels 1804, the channel seals can therefore define fluid pathways for transferring the cooling fluid 603.

The rear fluid channels 1804 belong to respective fluid circuits. More particularly, some channels and ports may belong to the upper fluid circuit 1702, and other channels and ports may belong to a lower fluid circuit 1704. The fluid channels and outlets on the rear plate surface 1802 can interconnect with the fluid channels and outlets on the front plate surface 1606 through the fluid ports 1706. More particularly, each fluid port 1706 can extend through the fluid transfer plate 1602 to interconnect the front fluid channels 1604 and ports to the rear fluid channels 1804 and ports. Similarly, given that the fluid channels and ports of the fluid transfer plate 1602 are connected to fittings on the fore plate 1502, which are in turn connected to the syringe barrel 408 through the conduit 1406, then the front fluid channel 1604, the rear fluid channel 1804, and the fluid ports 1706 are in fluid communication with the syringe cavity 1404. Accordingly, cooling fluid 603 can be moved between the syringe cavity 1404 and the channels in the fluid transfer plate 1602. Similarly, cooling fluid 603 can be moved between other external components and the channels in the fluid transfer plate 1602.

The outlets in the rear plate surface 1802 are labeled in FIG. 30 to show correspondence to the outlets in the front plate surface 1606 of FIG. 29. It is therefore apparent that the labeled outlets extend through the plate from the front plate surface 1606 to the rear plate surface 1802. More particularly, in the upper fluid circuit 1702, the U1 outlet 1722 and the U2 outlet 1724 are through-holes that extend through the plate. Similarly, in the lower fluid circuit 1704, the L1 outlet 1712, the L4 outlets 1718, and the L5 outlet 1720 are through-holes that extend through the plate. Accordingly, outlets that are isolated from each other on the front plate surface 1606 may be interconnected through the rear plate surface 1802. For example, the U1 outlet 1722 and the U2 outlet 1724 are physically isolated on the front plate surface 1606, however, those outlets are interconnected through the rear fluid channel 1804 on the rear plate surface 1802. Similarly, the L1 outlet 1712 and L5 outlet 1720 are physically isolated on the front plate surface 1606, however, those outlets are interconnected through the rear fluid channel 1804 on the rear plate surface 1802.

Whereas the fluid channels can interconnect outlets on one side of the plate that are isolated from each other on the other side of the plate, fluid ports 1706 can be used to reversibly interconnect fluid channels on one side of the plate with fluid channels on the other side of the plate. In an embodiment, each fluid port 1706 can be located within a corresponding valve seat on the rear plate surface 1802. The valve seats are labeled for ease of reference in the valve actuation logic described below. The upper fluid circuit 1702 can include a V1 valve seat 1730. The V1 valve seat 1730 can receive a corresponding piston 1610 to open and close the fluid port 1706 that interconnects the front fluid channel 1604 of the upper fluid circuit 1702 with the rear fluid channel 1804 of the upper fluid circuit 1702 at that location. Accordingly, the fluid port 1706 corresponding to the V1 valve seat 1730 can allow or stop fluid transfer between the front fluid channel 1604 and the rear fluid channel 1804 of the upper fluid circuit 1702. Thus, the fluid port 1706 corresponding to the V1 valve seat 1730 can cause the U1 and U2 outlets 1724 to be isolated from, or interconnected with, the U3 and U4 outlets 1728.

In an embodiment, the lower fluid circuit 1704 includes several valves seats. A V2 valve seat 1732 can receive a corresponding piston 1610 to open and close the fluid port 1706 that interconnects the front fluid channel 1604 of the lower fluid circuit 1704 with a first rear fluid channel 1804 of the lower fluid circuit 1704. The first rear fluid channel 1804 can interconnect the L1 outlet 1712 to the L5 outlet 1720. Accordingly, the fluid port 1706 corresponding to the V1 valve seat 1730 can allow or stop fluid transfer between the front fluid channel 1604 and the first rear fluid channel 1804 of the lower fluid circuit 1704. Thus, the fluid port 1706 corresponding to the V2 valve seat 1732 can cause the L2 and L3 outlets 1716 to be isolated from, or interconnected with, the L1 and L5 outlets 1720.

In an embodiment, a V3 valve seat 1734 can receive a corresponding piston 1610 to open and close the fluid port 1706 that interconnects the front fluid channel 1604 of the lower fluid circuit 1704 with a second rear fluid channel 1804 of the lower fluid circuit 1704. The second rear fluid channel 1804 can interconnect the fluid port 1706 at the V3 valve seat 1734 to the L4 outlets 1718. Accordingly, the fluid port 1706 corresponding to the V3 valve seat 1734 can allow or stop fluid transfer between the front fluid channel 1604 and the second rear fluid channel 1804 of the lower fluid circuit 1704. Thus, the fluid port 1706 corresponding to the V3 valve seat 1734 can cause the L2 and L3 outlets 1716 to be isolated from, or interconnected with, the L4 outlets 1718. It will also be appreciated, by examination of the illustrated fluid network, that actuating the pistons 1610 to simultaneously open the fluid ports 1706 at the V2 valve seat 1732 and the V3 valve seat 1734 would therefore place all of the outlets of the lower fluid circuit 1704 in fluid communication with each other through the front fluid channel 1604, the first rear fluid channel 1804, and the second rear fluid channel 1804.

As described above, the fluid network formed by the various channels and ports of the fluid transfer plate 1602 can be used to interconnect various components external to the cartridge manifold 1402. An embodiment of external component connections is now described. Beginning with the upper fluid circuit 1702, the U1 outlet 1722 can connect to the second syringe barrel 412. Accordingly, transferring fluid through the U1 outlet 1722 can transfer fluid to or from the second syringe barrel 412. The U2 outlet 1724 can connect to the fluid reservoir. Accordingly, transferring fluid through the U2 outlet 1724 can transfer fluid to or from the fluid reservoir. The U3 outlet 1726 can connect to a pressure sensor. Accordingly, the U3 outlet 1726 can allow the fluid pressure in the front fluid channel 1604 (or the rear fluid channel 1804 of the upper fluid circuit 1702 when the corresponding valve is opened) to be sensed. The U4 outlet 1728 can connect to an outlet line of the balloon catheter 101. Accordingly, transferring fluid though the U4 outlet 1728 can transfer fluid to or from the outlet line of the balloon catheter 101.

With respect to the lower fluid circuit 1704, the L1 outlet 1712 can connect to an inlet line of the balloon catheter 101. Accordingly, transferring fluid through the L1 outlet 1712 can transfer fluid to or from the inlet line of the balloon catheter 101. The L2 outlet 1714 can connect to the syringe barrel 408. Accordingly, transferring fluid through the L2 outlet 1714 can transfer fluid to or from the syringe barrel 408. The L3 outlet 1716 can connect to a pressure sensor. Accordingly, the L3 outlet 1716 can allow the fluid pressure in the front fluid channel 1604 (or one or both of the rear fluid channels 1804 of the lower fluid circuit 1704 when the corresponding valves are opened) to be sensed. The L4 outlets 1718 can connect to the fluid reservoir. Accordingly, transferring fluid though the L4 outlets 1718 can transfer fluid to or from the fluid reservoir. The L5 outlet 1720 can connect to a pressure sensor. Accordingly, the L5 outlet 1720 can allow the fluid pressure in the first rear fluid channel 1804 of the lower fluid circuit 1704 (or one or both of the front fluid channel 1604 or the second rear fluid channel 1804 of the lower fluid circuit 1704 when the corresponding valves are opened) to be sensed.

Having described the fluid network and, in an embodiment, the external components connected to the fluid network, it is now possible to describe a method of circulating cooling fluid 603 from the fluid reservoir to the balloon catheter 101 and then back to the fluid reservoir. In a first operation, the fluid port 1706 at the V2 valve seat 1732 may be closed and the fluid port 1706 at the V3 valve seat 1734 may be opened. This closing/opening action can be produced by actuation of the pistons 1610, as described below. Alternatively, other valve designs may be integrated with the fluid transfer plate 1602 to open and close the respective fluid ports 1706.

At a first operation, with the V3 valve open, the L2, L3, and L4 outlets may be in fluid communication with each other, and the L1 and L5 outlets may be isolated from the other outlets in the lower fluid circuit 1704. Accordingly, the syringe piston 702 of the syringe barrel 408 may be retracted to draw fluid into the syringe cavity 1404 from the fluid reservoir. More particularly, the cooling fluid 603 can pass from the fluid reservoir into the L4 outlet 1718, through the fluid port 1706 at the V3 valve seat 1734, into the front fluid channel 1604, and out of the L2 outlet 1714 into conduit 1406 connected to the syringe barrel 408. At this stage, the pressure sensor connected to the L3 outlet 1716 can sense pressure of the transferred cooling fluid 603, e.g., in the syringe cavity 1404.

At a second operation, the V3 valve is closed and the V2 valve is opened. At this stage, the L1, L2, L3, and L5 outlets can be in fluid communication with each other, and the L4 outlet 1718 may be isolated from the other outlets in the lower fluid circuit 1704. Accordingly, the syringe piston 702 of the syringe barrel 408 can be advanced to push fluid out of the syringe cavity 1404 into the inlet line of the balloon catheter 101. More particularly, the cooling fluid 603 can pass from the syringe cavity 1404 into the L2 outlet 1714, through the fluid port 1706 at the V2 valve seat 1732, and out of the L1 outlet 1712 into the inlet line of the balloon catheter 101. At this stage, the pressure sensor connected to the L5 outlet 1720 can sense pressure of the transferred cooling fluid 603, e.g., in the balloon catheter 101.

At a third operation, with the V1 valve open, the U1, U2, U3, and U4 outlets may be in fluid communication with each other. Accordingly, the syringe piston 702 of the second syringe barrel 412 may be retracted to draw fluid into the syringe cavity 1404 from the outlet line of the balloon catheter 101. More particularly, the cooling fluid 603 can pass from the outlet line of the balloon catheter 101 into the U4 outlet 1728, through the front fluid channel 1604 and the fluid port 1706 at the V1 valve seat 1730, into the rear fluid channel 1804, and out of the U1 outlet 1722 into conduit 1406 connected to the second syringe barrel 412. The conduit 1406 connecting the U2 outlet 1724 to the fluid reservoir may have a one-way check valve that prevents backflow, and thus, no suction may be applied to the fluid reservoir at the U2 outlet 1724. At this stage, the pressure sensor connected to the U3 outlet 1726 can sense pressure of the transferred cooling fluid 603, e.g., in the syringe cavity 1404.

At a fourth operation, the V1 valve is closed. At this stage, the U1 and U2 outlets 1724 can be in fluid communication with each other, and the U3 and U4 outlets 1728 may be isolated from the other outlets in the upper fluid circuit 1702. Accordingly, the syringe piston 702 of the second syringe barrel 412 can be advanced to push fluid out of the syringe cavity 1404 into the fluid reservoir. More particularly, the cooling fluid 603 can pass from the syringe cavity 1404 into the U1 outlet 1722, through the rear fluid channel 1804 of the upper fluid circuit 1702, and out of the U2 outlet 1724 through the conduit 1406 (and the check valve) to fill the fluid reservoir.

The operations described above can be performed in series and/or in parallel to circulate cooling fluid 603 through the balloon catheter 101. For example, adding fluid to the balloon at the second operation can be performed simultaneously with removing fluid from the balloon at the third operation to balance positive and negative pressures in the balloon such that the balloon diameter remains constant while maintaining a temperature of the cooling fluid 603 within the balloon. Control of the operations can be provided in part based on pressure data fed back to one or more processors by the pressure sensors connected to the cartridge manifold 1402.

Referring to FIG. 31, a perspective view of a piston of a cartridge manifold is shown in accordance with an embodiment. The valves used to open and close the fluid ports 1706 can include the pistons 1610. More particularly, the pistons 1610 can interact with the fluid transfer plate 1602 to seal and unseal the fluid ports 1706. In an embodiment, the piston 1610 include an end seal 1902. As described below, the piston 1610 can be placed in an open position in which the end seal 1902 unseals (does not occlude) a corresponding fluid port 1706 to allow cooling fluid 603 to pass through the fluid port 1706. The piston 1610 can be moved from the open position to a closed position in which the end seal 1902 seals (occludes) the corresponding fluid port 1706 to stop the cooling fluid 603 from passing through the fluid port 1706. Accordingly, the piston 1610 acts as a valve by covering or uncovering the fluid port 1706 to control fluid flow therethrough.

In an embodiment, the end seal 1902 has a circular distal surface. The distal surface can be flat. The seal can include an elastomeric, cylindrical plug that is lodged into a body of the piston 1610. A face of the plug can extend distally from the body to seal against an opposing surface. For example, the end seal 1902 can press against the rear plate surface 1802 of the fluid transfer plate 1602. More particularly, the face of the end seal 1902 can seal against the rear plate surface 1802 at a corresponding valve seat surrounding the corresponding fluid port 1706 to close the valve. Accordingly, the end seal 1902 can be sized to be larger than the fluid port 1706. For example, a diameter of the face of the end seal 1902 may be larger than, e.g., twice as large as, a diameter of the fluid port 1706.

As described above, the piston 1610 can be spring-loaded. The piston 1610 may include a spring groove 1904. The spring groove 1904 can include an annular groove sized and shaped to receive a proximal end of the spring 1612. The spring 1612 can be a helical compression spring 1612. A distal end of the spring 1612 can be similarly engaged to a corresponding spring groove 1904 at the valve seat. The spring grooves 1904 can stabilize the spring 1612, and allow the spring 1612 to act against both the rear plate surface 1802 and the piston 1610. Accordingly, the spring 1612 can bias the piston 1610 outward to maintain the piston 1610 in a normally open position in which the end seal 1902 is offset from the rear plate surface 1802 to allow fluid flow through the fluid port 1706.

The piston 1610 can include a side seal 1906 to seal against one of the manifold plates. For example, the side seal 1906 can seal against the aft plate 1504. Accordingly, the piston 1610 can include the end seal 1902 to press against the rear plate surface 1802 of the fluid transfer plate 1602, and a side seal 1906 to seal against the aft plate 1504. In an embodiment, the side seal 1906 can include an O-ring that fits within a groove of the piston 1610 body. Thus, the end seal 1902 may have an annular distal surface. The O-ring can extend laterally beyond a cylindrical wall of the body, and thus, when the piston body is inserted into a receiving hole of the aft plate 1504, the side seal 1906 can press against and seal to the aft plate 1504. The side seal 1906 can maintain the seal while sliding against the aft plate 1504, and thus, the piston 1610 may be moved axially within the aft plate 1504. Accordingly, the piston 1610 can be advanced to occlude the corresponding fluid port 1706 or retracted to open the corresponding fluid port 1706.

Referring to FIG. 32, a perspective view of a piston of a cartridge manifold is shown in accordance with an embodiment. The end seal 1902 may include an O-ring. The O-ring may be set within a groove in an end of the piston 1610. For example, the groove can be machined and the O-ring may be pressed into the groove. Alternatively, to create a more secure hold of the O-ring, the piston 1610 body may be overmolded around the O-ring. Accordingly, the end seal 1902 may be tightly secured within the piston 1610 body. In either case, the end seal 1902 can extend distally from the piston 1610 body such that the seal can press against the rear plate surface 1802 when the piston 1610 is moved to the closed position. An outer diameter of the annular end seal 1902 can be sized to be larger than the fluid port 1706. For example, the outer diameter of an O-ring end seal 1902 may be larger than, e.g., twice as large as, a diameter of the fluid port 1706.

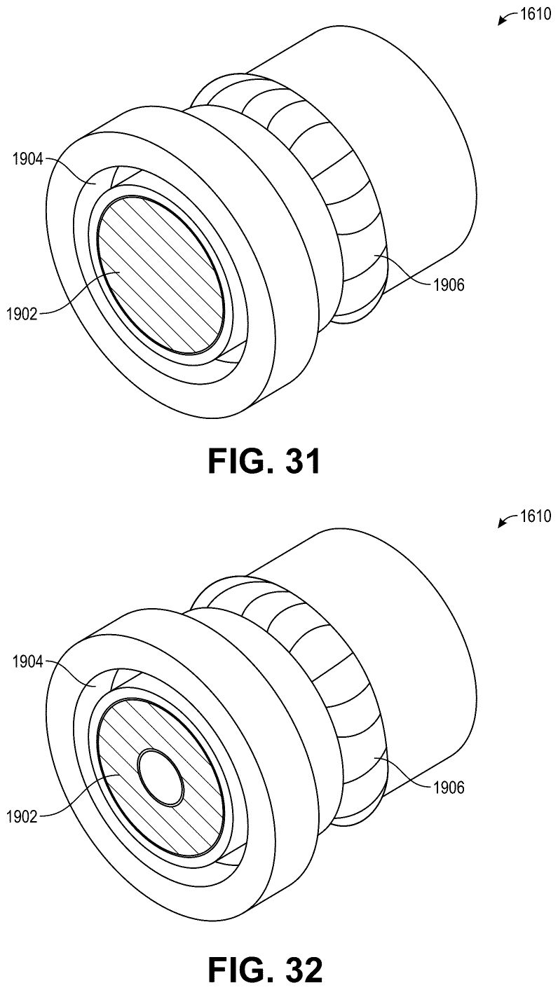

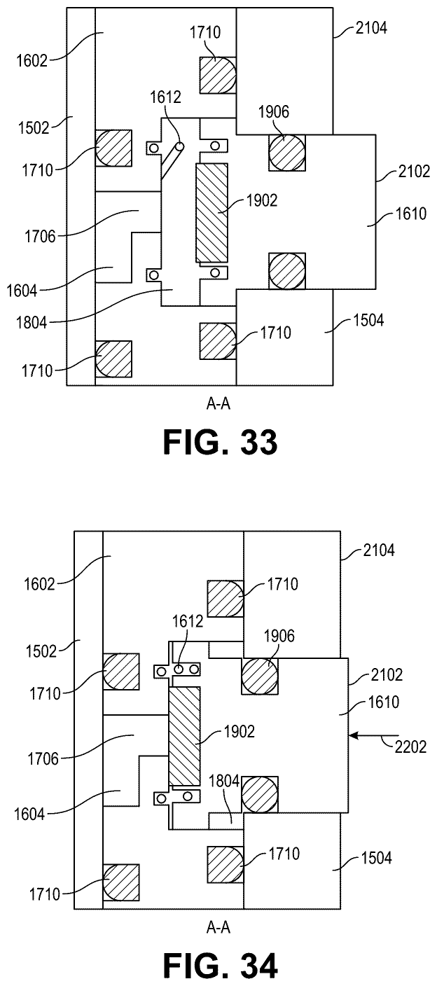

Referring to FIG. 33, a sectional view, taken about line A-A of FIG. 30, of a piston of a cartridge manifold in an open position is shown in accordance with an embodiment. The piston 1610 can be a free floating piston 1610 having a side seal 1906 to radially seal against the aft plate 1504, as described above. Furthermore, the end seal 1902 can face the fluid port 1706 in the fluid transfer plate 1602. In the open position, however, the spring 1612 can maintain the end seal 1902 spaced apart from the fluid port 1706. Furthermore, the fluid pressure within the fluid channels in front of the end face 1852 can press against the end face 1852, biasing the piston 1610 to the open position. Accordingly, cooling fluid 603 may flow through the front fluid channel 1604 and the fluid port 1706 into the rear fluid channel 1804.

Referring to FIG. 34, a sectional view, taken about line A-A of FIG. 30, of a piston of a cartridge manifold in an closed position is shown in accordance with an embodiment. A solenoid 2202 (force vector shown, but solenoid 2202 omitted) can be actuated to press the piston 1610 forward. The solenoid 2202 force can overcome the spring 1612 and fluid pressure acting against the piston 1610 in an opposite direction to cause the piston 1610 to move to the closed position. In the closed position, the end seal 1902 obstructs the path of fluid flow through the fluid port 1706. More particularly, the cooling fluid 603 is stopped from flowing through the fluid port 1706 to or from the front fluid channel 1604.

Notably, the solenoid 2202 can close the valve using a force of less than 10 lbf, e.g., 5 lbf or less. Such closing force compares favorably relative to alternative valve designs, such as pinch valves that squeeze conduit 1406 tubing. As a result, the cartridge manifold 1402 can also be designed to withstand lower compression forces, allowing for less material to be used in the design and a more compact form factor to be achieved.

The valve can be reversibly moved from the closed position of FIG. 34 to the open position of FIG. 33 by de-energizing the solenoid 2202. When the solenoid 2202 is no longer energized, the compression spring 1612 can act on the piston 1610 to return the piston 1610 to the open position.

As illustrated in FIGS. 33-34, a rear piston surface 2102 of the piston 1610 can be rearward of a back surface 2104 of the aft plate 1504 in both the open position (FIG. 33) and the closed position (FIG. 34). By maintaining the rear piston surface 2102 proud of the back surface 2104 in both piston 1610 positions, contact between the solenoids 2202 and the pistons 1610 is facilitated. More particularly, a likelihood of the solenoids 2202 losing contact with the rear piston surface 2102 is reduced because the rear piston surface 2102 does not recess 1210 into the hole in the aft plate 1504, below the back surface 2104.

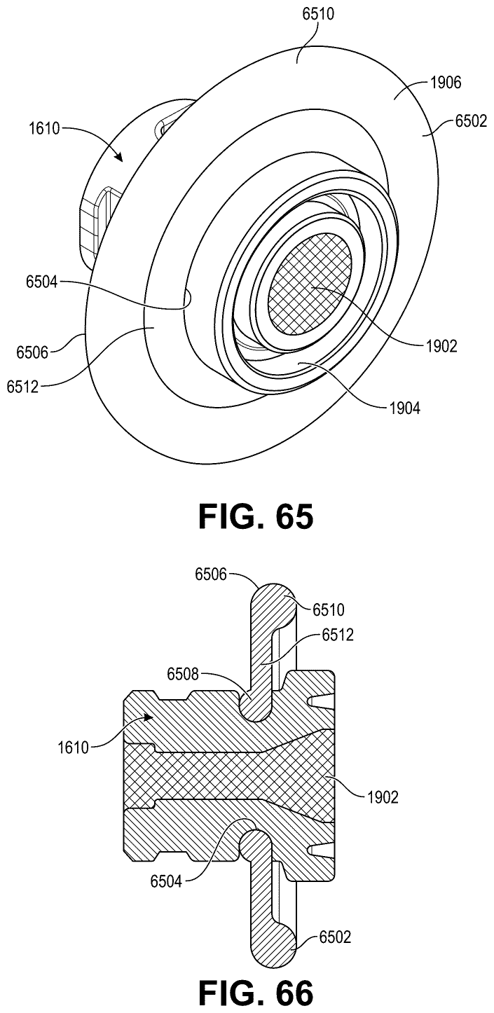

The piston embodiments described with respect to FIGS. 31-34 above are provided by way of example and not limitation. Other piston embodiments can be incorporated in a cartridge manifold to control fluid flow through a fluid port. For example, as described below with respect to FIGS. 65-67, a piston embodiment can include a diaphragm seal to mechanically constrain and seal a piston relative to a fluid transfer plate. More particularly, the piston can be sealed to a surrounding structure by a flexible flange-like seal that allows the piston to move axially relative to the fluid transfer plate to open and close a fluid port.

### Non-Invasive Pressure/Flow Sensor

As described above, pressure sensors used to monitor balloon inflation may be integrated directly within the system disposables, e.g., the fluid transfer cartridge 204. The pressure sensors are invasive, however, meaning that the pressure sensor contact the inflation fluid directly. As a result of the direct contact, the pressure sensors must be discarded after each procedure. The pressure sensors are expensive, however, so the current practice of using invasive pressure sensing drives up the per procedure cost.

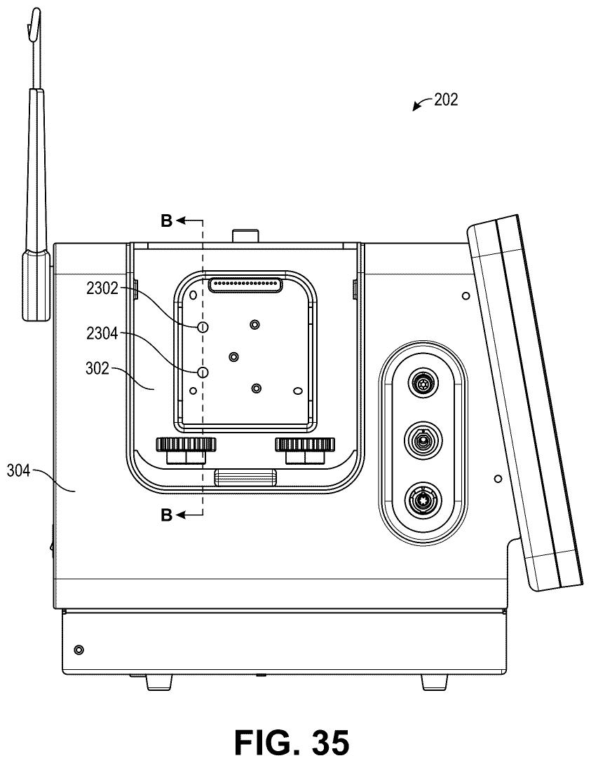

Referring to FIG. 35, a side view of a generator for an ultrasound-based treatment system is shown in accordance with an embodiment. Pressure and/or flow sensors can be integrated in the generator 202, rather than being incorporated in the fluid transfer cartridge 204. Furthermore, the generator-located pressure and/or flow sensors can be non-invasive, meaning that they do not directly contact the inflation fluid used to inflate the balloon. The non-invasive sensors can be used for multiple procedures, and thus, can reduce the cost of the disposable by removing costly sensors from the cartridge design.

In an embodiment, a pressure fitting 2302 is mounted on the generator housing 304. As described above, the generator housing 304 has the cartridge receptacle 302 configured to receive the fluid transfer cartridge 204. Accordingly, the pressure fitting 2302 can be behind the cartridge when the fluid transfer cartridge 204 is loaded into the cartridge receptacle 302 of the generator 202. The pressure fitting 2302 may be configured to connect to one or more conduits 1406 of the fluid transfer cartridge 204. For example, when the fluid transfer cartridge 204 is loaded into the generator 202, a fitting of the fluid transfer cartridge 204 that is connected to the conduit 1406 can engage the pressure fitting 2302 of the generator 202. In an embodiment, the pressure fitting 2302 may be connected to the cartridge manifold 1402, e.g., to the outlets of the manifold, through the conduit 1406. Thus, the pressure fitting 2302 may be used to transmit pressure from the cartridge manifold 1402 to the generator 202.

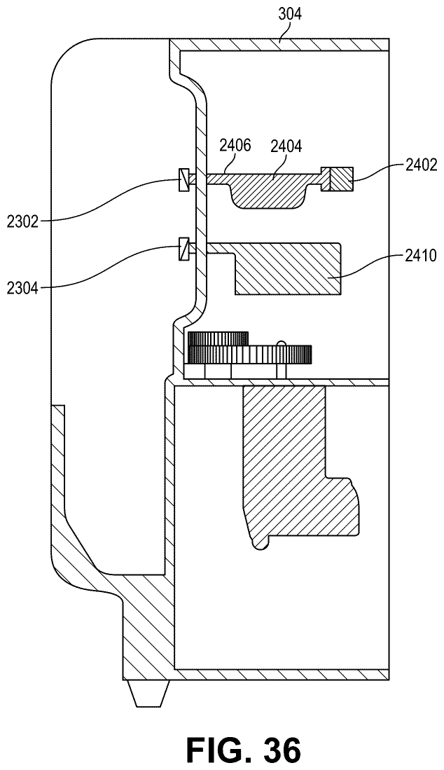

Referring to FIG. 36, a sectional view, taken about line B-B of FIG. 35, of a generator for an ultrasound-based treatment system is shown in accordance with an embodiment. In an aspect, the generator 202 incorporates non-invasive pressure sensors 2402 to monitor and measure fluid being transferred through the fluid transfer cartridge 204, e.g., being delivered to the balloon. In an embodiment, the generator 202 includes a pressure sensor 2402 within the generator housing 304. The pressure sensor 2402, which is integrated within the generator 202, is separate from the fluid transfer cartridge 204. The pressure sensor 2402 may be configured to sense pressure at the pressure fitting 2302. Thus, the pressure sensor 2402 may be used to measure fluid pressure being delivered to the balloon through the fluid transfer cartridge 204. Nonetheless, the pressure sensor 2402 may remain within the generator 202 (and re-used) when the cartridge is removed and discarded.

The generator-located pressure sensor 2402, which may replace the cartridge-located pressure sensors described above, can sense the inflation fluid non-invasively. For example, the pressure fitting 2302 may have a diaphragm that contacts the cooling fluid 603, but separates the cooling fluid 603 from the generator cavity. The pressure sensor 2402 may therefore connect to fluid lines that fluidly connect, e.g., tee into, the fluid lines in the cartridge, however, the pressure and/or flow sensors in the generator 202 may be isolated from the fluid in the cartridge. Cooling fluid 603 being fed to the balloon may therefore be sensed without being touched by the pressure sensor 2402. The sensors in the generator 202 can effectively monitor the fluid being fed to the balloon without becoming contaminated. Accordingly, the pressure sensor 2402 is non-invasive and re-usable, and thus, can reduce the cost of the disposable portions of the treatment system 100.

In an embodiment, the diaphragm of the pressure fitting 2302 acts on a fluid, such as air, which is in a line between the pressure fitting 2302 and the pressure sensor 2402. For example, a chamber, e.g., an air chamber 2404, can intervene between the pressure fitting 2302 (and thus, the fluid lines in the cartridge) and the fluid lines in the generator 202. The chamber can have a chamber inlet 2406 connected to the pressure fitting 2302 and a chamber outlet 2408 connected to the pressure sensor 2402. The air in the chamber can compress when the diaphragm is acted upon by the fluid in the cartridge, and thus, the air pressure can change with changes in the balloon inflation pressure. The changes may be sensed by the pressure sensor 2402.

Several types of non-invasive sensors are contemplated, as described further below. In an alternative embodiment, the chamber is a fluid chamber, which rather than being filled with air, may be filled with a liquid. More particularly, the chamber and/or lines between the diaphragm of the pressure fitting 2302 and the pressure sensor 2402 may be filled with an incompressible fluid. Such incompressible fluid may be acted upon by the diaphragm to relay pressure from the fluid transfer cartridge 204 to the generator 202 for non-invasive sensing.

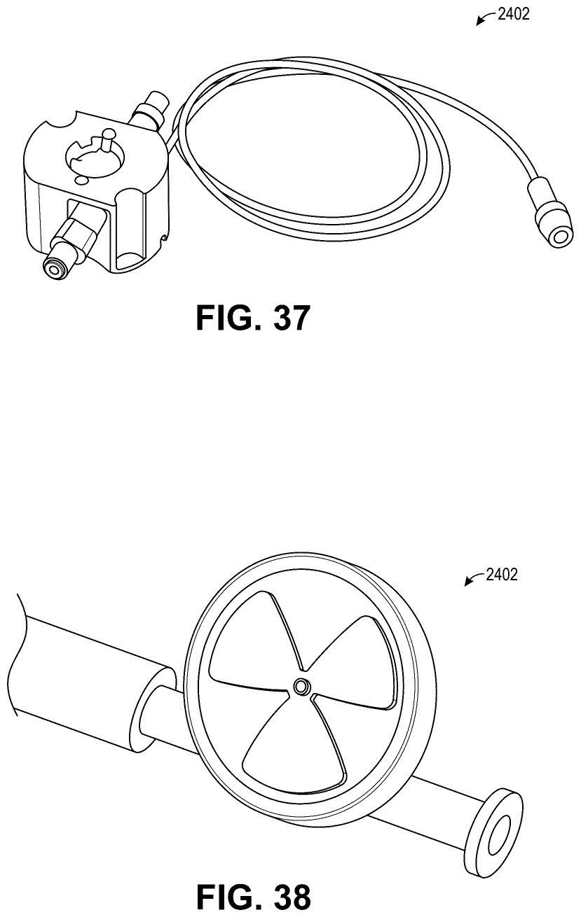

Referring to FIG. 37, a perspective view of a non-invasive sensor is shown in accordance with an embodiment. The pressure sensor 2402 may instead be a flow sensor. The non-invasive pressure and/or flow sensor in the generator 202 may be an ultrasonic sensor. Such sensors can use an ultrasonic signal that is directed into the fluid lines in the generator 202 to detect a Doppler shift of a reflected signal. A processor of the generator 202 can receive the sensed signals and determined, based on the Doppler shift, a flow of fluid within the fluid line. The sensor is non-invasive because the ultrasonic sensor does not contact fluid being fed to the balloon and the information is communicated thru a plastic tube.

Referring to FIG. 38, a perspective view of a non-invasive sensor is shown in accordance with an embodiment. The non-invasive fluid sensor can include a rotatable element mounted in a housing having an inlet and an outlet. The inlet and outlet can be connected to a fluid line within the generator 202, which is in turn in fluid communication with a fluid line within the cartridge. As fluid flows through the housing, vanes of the rotatable element will be driven. An optical sensor mounted outside of the housing can detect, through a housing wall, a speed of the vanes. The sensed signal may be provided to a processor of the generator 202 to determine, based on the vane movement, a pressure and/or flow of fluid within the fluid lines.

It will be appreciated that other non-invasive sensor types may be used. For example, a motor 1108 may drive a plunger of the cartridge to deliver and retrieve fluid from the balloon. The force required to drive the motor 1108, or a torque output of the motor 1108, may be sensed. The sensed motor parameters (either input or output parameters) can be used by a processor of the generator 202 to determine pressure and/or flow of fluid being transferred to the balloon without actually requiring the sensor to touch the fluid.

In an embodiment, a force sensor can detect force applied to the sensor by the fluid line. For example, the fluid line can include a compliant tubing portion that can be disposed against the force sensor. As pressure increases or decreases within the compliant tubing, the force applied to the sensor will increase or decrease because the tubing wall will expand or contract. The sensed force can be provided to a generator processor to determine, based on the force, a pressure or flow of fluid within the fluid line. The force sensor does not contact the fluid directly, and thus, is a non-invasive sensor that can be used in multiple procedures.

The use of non-invasive sensors in the generator 202 to monitor fluid delivery to/from the balloon, rather than using invasive sensors in the fluid transfer cartridge 204, allows for: the cost of manufacturing the cartridge to be reduced, fluid monitoring sensors to be used for multiple procedures, and thus, a reduction in the per procedure cost.

### Pneumatic Syringe Drive

As described above, the fluid drive system may be mechanically-driven. More particularly, the drive system can include a stepper motor, and a transmission having several gears and a worm screw. The mechanical system components may, however, increase the cost and space requirements of the system. Furthermore, the drive system may be complex.

The mechanically-driven fluid drive system may be replaced by a pneumatically-driven fluid drive system. More particularly, the screw drive can be replaced by pneumatic pressure lines. Whereas the screw drive moves the shaft of the syringes by gearing that advances the worm screw, the pneumatic pressure lines can advance/retract the stoppers 608 using positive and negative pressure.

Referring again to FIG. 36, the system can integrate a pneumatic drive system 2410 to advance and/or retract stoppers 608 of the syringes that feed inflation fluid to the balloon. The generator 202 can include a pneumatic fitting 2304 (FIG. 35) connected to the pneumatic drive system 2410. More particularly, the pneumatic fitting 2304 can be mounted on the generator housing 304.

The pneumatic fitting 2304 can be connected to a pneumatic drive system 2410. The pneumatic drive system 2410 can be in the generator housing 304. The pneumatic drive system 2410 may be configured to apply one or more of positive pressure or negative pressure to the pneumatic fitting 2304. For example, the pneumatic drive system 2410 can include a pneumatic pump and/or a vacuum pump that increases or decreases the pressure at the pneumatic fitting 2304.

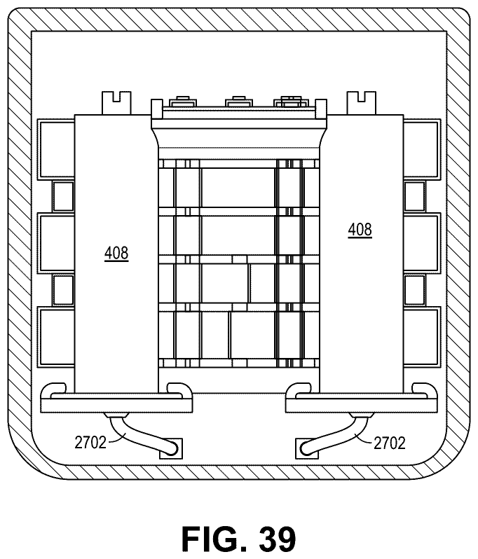

Referring to FIG. 39, a front view of an internal portion of a fluid transfer cartridge is shown in accordance with an embodiment. The pneumatic drive system 2410 can connect to a pressure line 2702 that connects to the syringe. The pressure line 2702, for example, can be a segment of tubing extending from the pneumatic fitting 2304 to a syringe connector that attaches to a base of the syringe barrel 408. By applying positive and negative pressure to the pressure line 2702, a stopper 608 of the syringe can be driven back and forth within the syringe barrel 408. More particularly, positive pressure delivered to the pressure line 2702 through the pneumatic fitting 2304 can drive the stopper 608 upward to advance cooling fluid 603 into a distal fluid line, and negative pressure applied to the pressure line 2702 through the pneumatic fitting 2304 can drive the stopper 608 downward to retract inflation fluid from the fluid line. The inflation fluid can therefore be delivered to and retrieved from the balloon during a procedure.

Referring to FIG. 40, a sectional view of an internal portion of a fluid transfer cartridge having a pneumatically driven syringe is shown in accordance with an embodiment. The syringes can include respective stoppers 608. The stopper 608 can be disposed within the syringe barrel 408 between a distal syringe cavity 2802 and a proximal syringe cavity 2804. The proximal syringe cavity 2804 is in fluid communication with the pneumatic fitting 2304, and the distal syringe cavity 2802 is in fluid communication with the fluid network distal to the syringe. As described above, the fluid network can include the fluid reservoir. Thus, the distal syringe cavity 2802 can be in fluid communication with the fluid reservoir, e.g., via the cartridge manifold 1402. When the pneumatic fitting 2304 delivers positive pressure to the proximal syringe cavity 2804, the stopper 608 advances to expel cooling fluid 603 from the distal syringe cavity 2802. When the pneumatic fitting 2304 draws vacuum from the proximal syringe cavity 2804, the stopper 608 retracts to draw cooling fluid 603 into the syringe barrel 408.

The use of a pneumatic drive system 2410 simplifies the fluid drive design. The pneumatic system requires fewer parts in the disposable because it replaces a gear and worm screw with a simple pressure line 2702. Accordingly, complexity and cost of the system can be reduced.

In an embodiment, the syringes may be totally removed from the cartridge and relocated next to the fluid reservoir. Alternatively, the syringes could be provided as part of an assembly including the syringes and the fluid reservoir, which can then be connected to the generator 202. In either case, it may be possible to eliminate the cartridge entirely, since the fluid transfer function can be performed directly by the generator 202. In such case, the generator 202 may have inlet/outlet fluid lines that transfer fluid directly from the fluid reservoir to the balloon without being transferred through a cartridge. The generator 202 can include pressure lines 2702 that connect to the syringes, and the syringes can receive/output fluid directly to the reservoir. The generator 202 can include pinch valves that connect to the syringes, and the syringes can receive/output fluid directly to the reservoir. Accordingly, transitioning from a mechanical to a pneumatic drive paradigm has the potential to substantially reduce complexity and cost of the treatment system 100.

### Non-Contact Syringe Position Sensing

Positioning of the syringe pistons 702 can be sensed by switches, as described above. More particularly, mechanical switches and/or magnetic switches may be used to detect a position of a piston end. Position feedback may be used by one or more processors of the generator 202 to detect and/or determine when the syringes are empty or full. Mechanical switches may be prone to failure. Furthermore, such switches require precise placement in the generator 202 to provide accurate data. Magnetic switches tend to offer low positional resolution. A precision of such switches is therefore wanting. Accordingly, the treatment system 100 may benefit from position-sensing components that are robust, durable, and accurate. The system can also benefit from information about the syringe piston 702 position over an entire stroke, rather than only at an empty or full position.

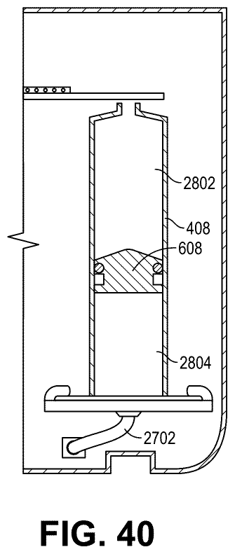

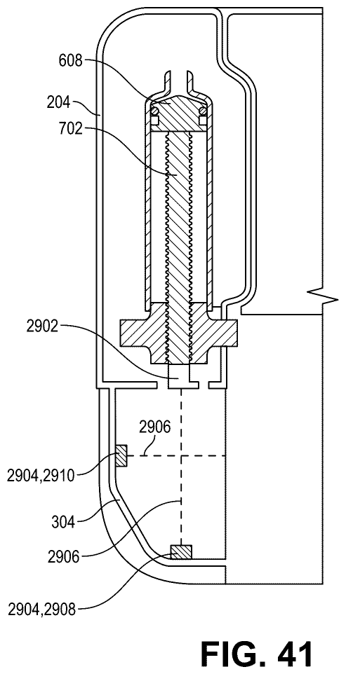

Referring to FIG. 41, a sectional view of an internal portion of a fluid transfer cartridge having a non-contact position sensor is shown in accordance with an embodiment. The generator 202 can include a non-contact position sensor 2904 mounted in the generator housing 304. The non-contact position sensor 2904 may be configured to detect a position of the syringe piston 702. More particularly, the non-contact position sensor 2904 can be positioned and oriented such that a line of sight 2906 of the sensor is directed at a portion of the syringe that is connected to the stopper 608. For example, the sensor can direct radiation, e.g., light, toward the piston and sense reflected radiation that indicates the position of the piston end 2902.

In an embodiment, the non-contact position sensor 2904 includes a time-of-flight sensor 2908. The time-of-flight sensor 2908 may be directed parallel to a central axis of the syringe and/or syringe piston 702. For example, the time-of-flight sensor 2908 can be mounted on a bottom wall of the generator housing 304 facing upward toward the syringe. Accordingly, the sensor can be directed in a longitudinal direction, which is a direction of shaft movement.

The time-of-flight sensor 2908 can emit radiation toward the piston end 2902, and some amount of the radiation may be reflected by the piston 1610 back to the time-of-flight sensor 2908. The reflected signal can be processed by one or more processors of the generator 202 to determine a distance between the time-of-flight sensor 2908 and the piston end 2902. More particularly, a time that the radiation took to move to the piston end 2902 and bounce back to the sensor can be measured and used to determine the distance. Based on known geometrical relationships between the piston end 2902 and the stopper 608 of the syringe, information about a volume of cooling fluid 603 in the syringe can be determined.

In an embodiment, the non-contact position sensor 2904 includes a proximity sensor 2910. The proximity sensor 2910 may be directed parallel to the direction of shaft movement, as described above. However, in an embodiment, the proximity sensor 2910 has a line of sight 2906 that is orthogonal to the direction of shaft movement. For example, the non-contact position sensor 2904 can be mounted on a side wall of the generator housing 304, and may be directed radially through a cavity that receives the syringe piston 702 during syringe operation.

The proximity sensor 2910 can provide a go-no-go indication of whether the piston end 2902 has reached a predetermined location along the direction of movement. As the piston end 2902 moves downward to the location, within the cavity, the proximity sensor 2910 will detect the presence of the piston end 2902. More particularly, an intensity of reflected radiation that is sensed by the proximity sensor 2910 will change when the piston end 2902 passes through the line of sight 2906. Thus, the proximity sensor 2910 can detect the location of the piston end 2902. Several proximity sensors 2910 can be placed along the side wall to detect different locations of the piston end 2902 that correspond to fluid levels of the syringe. For example, several proximity sensors 2910 could sense piston locations corresponding to the syringe being full of the cooling fluid, half-full, and empty.

The syringe position data generated by the non-contact position sensor 2904 can be used to determine a volume of fluid delivered to the balloon catheter 101. The data may be continuous, e.g., over an entire stroke of the syringe, and therefore may provide an indication of the position of the syringe at every position along the stroke. Furthermore, the non-contact position sensors 2904 can be stably mounted on the generator housing 304 and their locations can be calibrated, thus, the position data can be accurate. Time-of-flight, proximity, and other types of non-contact position sensors 2904 are inexpensive, and thus, can be implemented at low cost.

It will be appreciated that alternative sensors and sensor placements may be used. For example, the non-contact position sensor 2904 can include an acoustic sensor, rather than a light sensor. Acoustic sensors emit and receive sound signals to determine presence and distance to a surface (such as the piston end 2902).

The position of the sensor may also be moved to any location within the generator 202. For example, a structure other than the generator housing 304 may provide a mounting location for the non-contact position sensor 2904. In an embodiment, the non-contact position sensor 2904 can be mounted on a structure other than the generator 202. For example, the sensor could be placed on the syringe shaft 704. In such case, the time-of-flight sensor 2908 could be mounted on the shaft end 706. The sensor may be directed in the direction of shaft movement to sense movement of the shaft 704 based on changing distance between the sensor and an adjacent surface, e.g., the generator housing 304.

The position of the sensor may also be moved to any location within the cartridge. For example, a structure other than the cartridge housing may provide a mounting location for the non-contact position sensor 2904. In an embodiment, the non-contact position sensor 2904 can be mounted on a structure other than the cartridge. For example, the sensor could be placed on the syringe shaft 704. In such case, the time-of-flight sensor 2908 could be mounted on the shaft end 706. The sensor may be directed in the direction of shaft movement to sense movement of the shaft 704 based on changing distance between the sensor and an adjacent surface, e.g., the cartridge housing.

### Fluid Reservoir Detection

The treatment system 100 includes a fluid reservoir containing the cooling fluid 603 that is circulated through the balloon catheter 101. The fluid reservoir can be a container holding the cooling fluid 603. For example, the container can be a bag containing the cooling fluid 603. The cooling fluid 603 may be selected based on the procedure and/or device that is being used. For example, some balloon catheters 101 may perform optimally with sterilized water, while others may function using saline. Accordingly, the presence and type of fluid reservoir, e.g., its volume and contents, is important to proper system performance. In an embodiment, the treatment system 100 is capable of detecting a presence and/or type of fluid reservoir that is being used during the procedure.

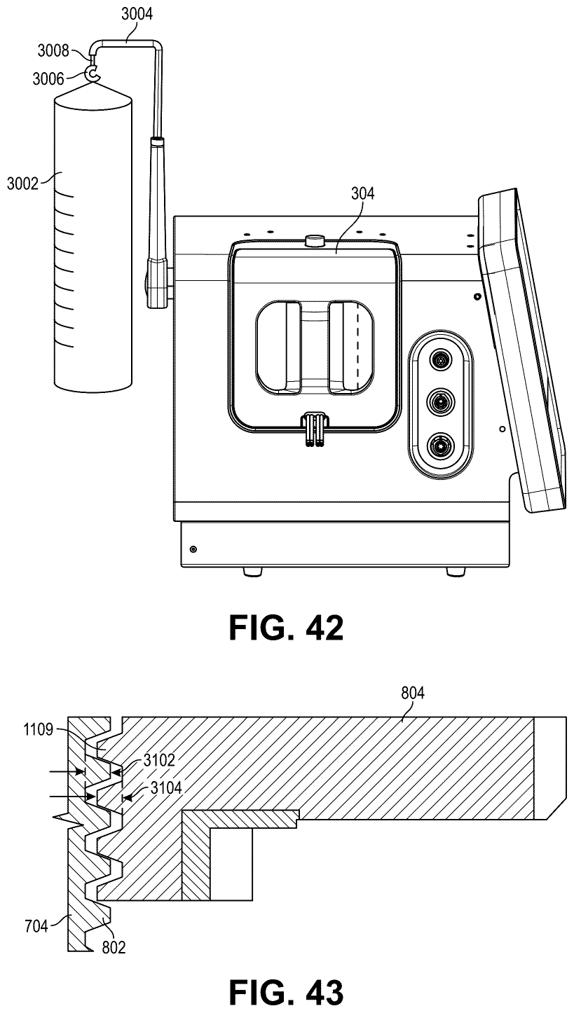

Referring to FIG. 42, a perspective view of an ultrasound-based treatment system is shown in accordance with an embodiment. The treatment system 100 can include a fluid reservoir holder 3004 to hold a fluid reservoir 3002. The fluid reservoir holder 3004 may have an attachment 3006, e.g., a hook, to hold the reservoir. For example, the fluid reservoir 3002 can be a sterilized-water filled bag having a loop that can be placed on the attachment 3006 to hang the bag from the fluid reservoir holder 3004.

In an embodiment, the system includes a sensor to sense a presence and/or characteristic of the fluid reservoir 3002. The characteristic may be a weight of the fluid reservoir 3002. For example, a weight sensor 3008 may be coupled to the attachment 3006 to generate weight data based on a weight of the fluid reservoir 3002. The weight sensor 3008 can be mounted within or on the generator housing 304. Alternatively, the weight sensor 3008 can be integrated with the fluid reservoir holder 3004. For example, the weight sensor 3008 can include a strain gauge having an end connected to the attachment 3006 and an end connected to a crossbar or upright of a bag suspension structure 3004. Accordingly, the strain gauge can be located at any location that undergoes tension, compression, or bending moments as a result of the fluid reservoir weight. Accordingly, the weight sensor 3008 can detect and/or measure physical strain resulting from such loads to generate data corresponding to a weight of the fluid reservoir 3002.

The generated data may be used by one or more processors to determine information about the fluid reservoir 3002. More particularly, the one or more processors can receive the weight data from the weight sensor 3008, and determine, based on the weight data, information corresponding to the fluid reservoir 3002.

In an embodiment, the one or more processors determine whether the fluid reservoir 3002 is present. Presence detection can be used to verify that the fluid reservoir 3002 is available at the right time in the procedure (or determine whether it is removed). The fluid reservoir 3002 may be essential to one or more procedure operations, such as preparing and inflating the balloon catheter 101. When the fluid reservoir 3002 is not present, e.g., when the fluid bag is not hung on the bag stand, those procedural operations can fail, which can undesirably prolong the procedure.

The one or more processors can determine presence of the fluid reservoir 3002 based on the weight data being above a predetermined weight threshold, or within a predetermined range of weight. The one or more processors can generate a presence signal based on the weight. The presence signal may be used as a gate to a logical sequence in the procedure. For example, the presence signal can allow the user interface to advance to subsequent operations in the preparation procedure, or else generate an error message to prompt the user to load or replace the fluid reservoir 3002.

In addition to being a logical gate, the weight data can be used as an interlock to other system components. For example, when the fluid reservoir 3002 is not present, the syringe drive system may be disabled to prevent operation when there is no cooling fluid 603 available to fill the syringe barrel 408. Presence is only one characteristic that can drive the above decisions. Other characteristics that can be sensed include a fluid reservoir type (including cooling fluid type) and/or leak detection.

In an embodiment, the one or more processors determine whether the fluid reservoir is a predetermined fluid reservoir. Weights of fluid reservoirs 3002 may be known based on a volume and density of the cooling fluid 603 stored in the reservoir. For example, a specific volume of saline may have a different weight than the same volume of sterilized water. Furthermore, fluid reservoirs may be made from different materials, e.g., vinyl or silicone, which can also affect the predetermined weight of the fluid reservoir 3002. The treatment system 100 may be calibrated or programmed with the known weights of specific fluid reservoirs. Accordingly, the weight data may be used by the one or more processors to determine whether a particular fluid reservoir having a specified cooling fluid volume and/or type is mounted on fluid reservoir holder 3004.

Certain balloon catheters 101 may use transducers 108 that perform optimally with sterile water as the cooling fluid 603. For example, using saline rather than sterile water with such transducers may cause the transducer to malfunction. Accordingly, the one or more processors can determine whether the fluid reservoir 3002 contains sterile water or saline, based on the weight of the bag. When the one or more processors determine that the fluid reservoir 3002 contains sterile water, the procedure may be allowed to proceed. Alternatively, if the bag contains saline, the one or more processors may generate the error signal and/or lockout operation of other system components to prevent damage to the transducer 108. For example, if the specific weight is not detected, the system can prompt the user to verify that sterile water (or dextrose, etc.) is being used. Accordingly, the weight sensor 3008 can be used to detect whether a correct fluid is being used based on the bag having a specific and unique weight.

In an embodiment, the one or more processors determine whether there is a leak in the fluid reservoir 3002. The weight data may be used to detect that the bag weight is changing during the procedure. More particularly, one or more processors can detect a change in the weight of the fluid reservoir 3002 during the procedure, which may indicate the loss of fluid due to a leak. In response to the leak detection, the system can generate an error message and/or prompt the user to verify that the fluid reservoir 3002 is not leaking, or to take another corrective action.

Referring to FIG. 43, a sectional view of a drive mechanism of a fluid transfer cartridge is shown in accordance with an embodiment. As described above with respect to FIG. 32, the drive mechanism includes the shaft 704 of the syringe piston 702 having an external thread 802, and the gear 804 having an internal thread 1109 engaging the external thread 802. In an embodiment, the external thread 802 and the internal thread 1109 are configured to avoid binding between the threads. Binding between the threads can occur, for example, when standard thread designs are used that do not allow for sufficient clearance between the crest and root of the engaged threads. More particularly, binding can occur more frequently when the working depth of the external thread 802 and the internal thread 1109 are equal. In an embodiment, the external thread 802 and the internal thread 1109 have different working depths. For example, the external thread 802 can have an external working depth 3102, and the internal thread 1109 can have an internal working depth 3104. The external working depth 3102 may be greater than the internal working depth 3104. For example, the external working depth 3102 can be at least 25%, e.g., 50%, greater than the internal working depth 3104. The different working depths allow the threads to engage securely without binding as the shaft 704 is driven by the gear 804.

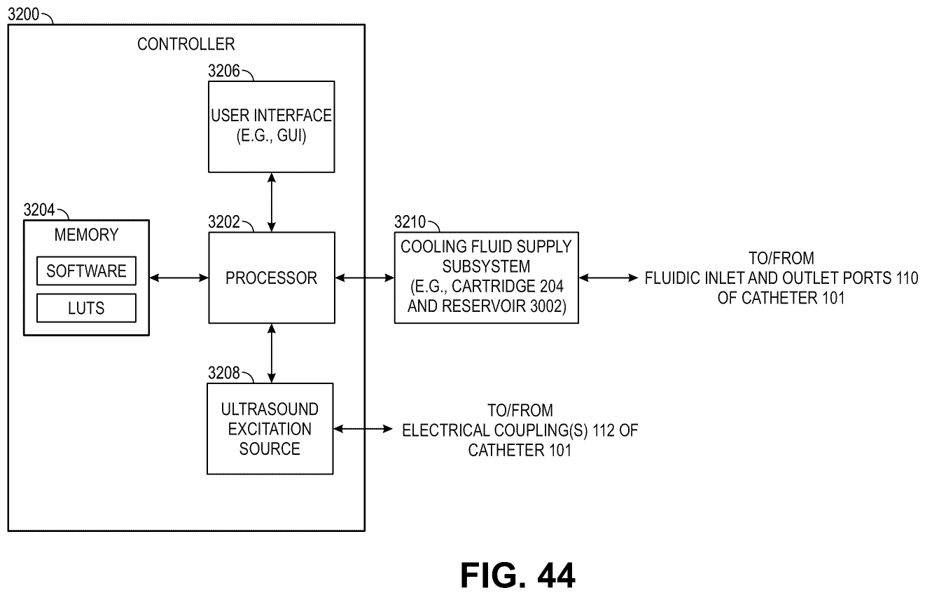

Referring to FIG. 44, a block diagram of a controller of a treatment system is shown in accordance with an embodiment. The block diagram represents an example implementation of the controller, which was introduced above. A controller 3200 is shown as including one or more processors 3202, a memory 3204, a user interface 3206, and an ultrasound excitation source 3208, but can include additional and/or alternative components. While not specifically shown, a processor 3202 can be located on a control board, or more generally, a printed circuit board (PCB) along with additional circuitry of the controller 3200. The processor 3202 can communicate with the memory 3204, which can include a non-transitory computer-readable medium storing instructions. The processor 3202 can execute the instructions to cause the treatment system 100 to perform the methods described herein. The user interface 3206 interacts with the processor 3202 to cause transmission of electrical signals at selected actuation frequencies to the ultrasound transducer 108 via wires of the connection cable and the cabling that extends through the catheter shaft 704. These wires electrically couple the controller 3200 to the transducer 108 so that the controller 3200 can send electrical signals to the transducer 108, and receive electrical signals from the transducer 108. The processor 3202 can control the ultrasound excitation source 3208 to control the amplitude and timing of the electrical signals so as to control the power level and duration of the ultrasound signals emitted by transducer 108. More generally, the controller 3200 can control one or more ultrasound treatment parameters that are used to perform sonication. In certain embodiments, the excitation source can also detect electrical signals generated by transducer 108 and communicate such signals to the processor 3202 and/or circuitry of a control board. While the ultrasound excitation source 3208 in FIG. 44 is shown as being part of the controller 3200, it is also possible that the ultrasound excitation source 3208 is external to the controller 3200 while still being controlled by the controller 3200, and more specifically, by the processor 3202 of the controller 3200.

The user interface 3206 can include a touch screen and/or buttons, switches, etc., to allow for an operator (user) to enter patient data, select treatment parameters, view records stored on a storage/retrieval unit (not shown), and/or otherwise communicate with the processor 3202. The user interface 3206 can include a voice-activated mechanism to enter patient data or may be able to communicate with additional equipment so that control of the controller 3200 is through a separate user interface 3206, such as a wired or wireless remote control. In some embodiments, the user interface 3206 is configured to receive operator-defined inputs, which can include, e.g., a duration of energy delivery, one or more other timing aspects of the energy delivery pulses (e.g., frequency, duty cycle, etc.), power, body lumen length, mode of operation, patient parameter, such as height and weight, and/or verification of artery diameter, or a combination thereof. Example modes of operation can include (but are not limited to): system initiation and set-up, catheter preparation, balloon inflation, verification of balloon apposition, pre-cooling, sonication, post-cooling, balloon deflation, and catheter removal, but are not limited thereto. In certain embodiments, the user interface 3206 provides a graphical user interface (GUI) that instructs a user how to properly operate the treatment system 100. The user interface 3206 can also be used to display treatment data for review and/or download, as well as to allow for software updates, and/or the like.

The controller 3200 can also control a cooling fluid supply subsystem 3210, which can include the fluid transfer cartridge 204 and fluid reservoir 3002, which were described above, but can include alternative types of fluid pumps, and/or the like. The cooling fluid supply subsystem 3210 is fluidically coupled to one or more fluid lumens (e.g., 110) within the catheter shaft which in turn are fluidically coupled to the balloon. The cooling fluid supply subsystem 3210 can be configured to circulate a cooling liquid through the catheter 101 to the transducer 108 in the balloon. The cooling fluid supply subsystem 3210 may include elements such as the fluid reservoir 3002 for holding the cooling fluid 603, pumps (e.g., syringes), a refrigerating coil (not shown), or the like for providing a supply of cooling fluid 603 to the interior space of the balloon at a controlled temperature, desirably at or below body temperature. The processor 3202 interfaces with the cooling fluid supply subsystem 3210 to control the flow of cooling fluid 603 into and out of the balloon. For example, the processor 3202 can control motor control devices linked to drive motors 1108 associated with pumps for controlling the speed of operation of pumps (e.g., syringes). Such motor control devices can be used, for example, where the pumps are positive displacement pumps, such as peristaltic pumps. Alternatively, or additionally, a control circuit may include structures such as controllable valves connected in the fluid circuit for varying resistance of the circuit to fluid flow (not shown). The processor 3202 can monitor pressure measurements obtained by the pressure sensors (e.g., P1, P2 and P3) to monitor and control the cooling fluid 603 through the catheter 101 and the balloon. The pressure sensors can also be used to determine if there is a blockage and/or a leak in the catheter 101. While the balloon is in an inflated state, the pressure sensors can be used to maintain a desired pressure in the balloon, e.g., at a pressure of between 10 psi and 30 psi, but not limited thereto. As will be described in additional detail below, the processor 3202 can use sensor measurements from one or more of the pressure sensors 2402 and/or other sensors to determine when the balloon is in apposition with a body lumen as well as to estimate an inner diameter of a body lumen in order to select an appropriate dose of ultrasound energy to be delivered to treat tissue surrounding the body lumen.

The controller 3200 can control operation of the generator 202 and fluid transfer cartridge 204 components to drive the inflation of the balloon prior to or during an interventional procedure. For example, the controller 3200 can control a priming process. The priming process can fill one or more of the syringes, the fluid manifold, the fluid conduit lines, and the balloon of the treatment system 100 with fluid, and remove bubbles from the system. More particularly, the priming process can purge air from the fluidic system and prepare the treatment system 100 for delivery into the patient. The priming process may, as described below, include expelling fluid from a return syringe before filling an injection syringe to avoid sending air into the injection syringe. The controller 3200 may also control the inflation procedure, as described above, by driving the syringe piston 702 vertically to move the stopper 608 within the syringe and thus draw fluid into or expel fluid out of the syringe.

A position of the stopper 608 within the syringe can be determined by the controller 3200 based on several sensor inputs. The controller 3200 can receive feedback from the motor 1108 that drives the syringe piston 702 to determine stopper position. For example, the motor 1108 can provide data corresponding to a number of rotations of the gear 804, and the controller 3200 can determine, based on the rotations and known thread pitch information, a distance that the stopper 608 has been moved within the syringe. Furthermore, as described above, a magnetic or optical sensor can detect a location of the shaft end 706, e.g., a home position 1102. When the shaft end 706 is at the home position 1102, the stopper 608 can be at a known location within the syringe.

Although the motor 1108 feedback and home position sensor(s) can provide precise determination of the home position 1102, system slippage in the gear teeth or motor 1108 can lead to some inaccuracy as to whether the stopper 608 is accurately located at a same home position 1102 after each inflation/deflation cycle. More particularly, as the piston is driven upward and downward within the syringe over several cycles, the shaft end 706 may be driven based on motor 1108 rotations to a different home position 1102 in which the homing sensor is not triggered. When this happens, an error may be generated by the system. However, there may be only a marginally different amount of fluid remaining in the syringe when the error is triggered, as compared to when the stopper 608 was at the original home position 1102, which could create a nuisance requiring the user to re-home the system even when there is no practical impact to the system efficacy.

To avoid such nuisances, a homing process can be used that will dynamically adjust the home position when changes in the home position do not negatively affect system operation, and to generate an error when the changes may negatively affect system operation.

In an operation, the fluid transfer cartridge 204 is loaded into the generator 202. When operation begins, the shaft end 706 of the fluid transfer cartridge 204 will either be detected by the homing sensor, or not. If the shaft end 706 is not detected, then the controller 3200 can determine that the syringe must be homed prior to proceeding with fluidic priming and/or balloon inflation/deflation. If the shaft end 706 is detected, then the syringe can already be determined to be homed.

In the first case, when the shaft end 706 is not initially detected, the controller 3200 can drive the motor 1108 to raise the syringe pistons 702 until the shaft end 706 is detected by the position sensors. This is the initial home position. The controller 3200 can set the encoder volumes to zero at the initial home position. More particularly, the controller 3200 can determine a position value of the motor 1108 encoders and the position value can be set as the initial home position (corresponding to the home position of shaft end 706).

Switches include always on, always off, and intermittent (between always on and always off) positions. In an embodiment, the shaft end 706, upon reaching the initial home position, can be in either the always on or intermittent positions. If the initial home position is in the always on position, then lowering and raising the shaft end 706 to the initial home position should trigger a home position sensor. If the initial home position is in the intermittent position, then lowering and raising the shaft end 706 to the initial home position may or may not trigger a home position sensor.

It will be appreciated that, by monitoring shaft end position and motor encoders, a comparison of shaft end 706 location and remaining fluid volume can be performed. For example, at any location, the motor encoder information can be used to determine a stopper position and, thus, how much cooling fluid 603 remains in the syringe. In an embodiment, when the home position sensor is triggered, the controller 3200 can determine a remaining fluid volume in the syringe. When the remaining fluid volume is less than a predetermined volume, e.g., 3 to 5 mL, when the shaft end 706 is detected by the position sensor, even if the motor encoder is not at the same location as the initial home position, then the controller 3200 may set the motor position as a new home position. Alternatively, if the remaining volume is greater than the predetermined volume, e.g., greater than 5 mL, then the controller 3200 may generate an error to require the user to troubleshoot and re-home the system. In either case, the motor encoder can be used to determine cooling fluid volumes within the syringe at all states of the priming and/or inflation/deflation processes.

In the second case, when the shaft end 706 is initially detected, the controller 3200 can drive the motor 1108 to lower the syringe piston 702 to draw a predetermined volume of cooling fluid 603, e.g., 3 to 5 mL, into the syringe. The home position sensor can be monitored during the lowering process. If the home position sensor turns off during the lowering movement, then the motor encoder position can be set as a new home position by the controller 3200. The controller 3200 may proceed to control the system to perform the priming and/or inflation/deflation processes. Alternatively, if the home position sensor is still on after lowering the syringe to draw the predetermined volume of cooling fluid 603 into the syringe, then the controller 3200 can generate an error to require the user to troubleshoot and re-home the system. More particularly, the sensor remaining on after the lowering of the syringe likely indicates that the sensor has malfunctioned and the user may be notified accordingly. In either case, the motor encoder can be driven to perform the priming and/or inflation/deflation processes.

Referring to FIG. 45, a perspective view of a shaft end having an optical tab is shown in accordance with an embodiment. As described above, the shaft end 706 can include a feature to trigger the position sensor. For example, the feature can be an optical tab 3302, also referred to as an optical feature. The optical tab 3302 can include a tab, prong, flag, etc., to trigger an optical sensor. In an embodiment, the optical tab 3302 extends radially outward from a shaft axis 3304. The shaft axis 3304 can be a longitudinal axis of the shaft 704. The radial extension can protrude outward such that light emitted by the optical sensor in a direction transverse to the shaft axis 3304 can reflect from the optical tab 3302 when the shaft end 706 is adjacent to the optical sensor. Accordingly, the optical tab 3302 can block the optical sensor to trigger the sensor and indicate to the controller 3200 that the shaft 704 and the stopper 608 are at a particular position.

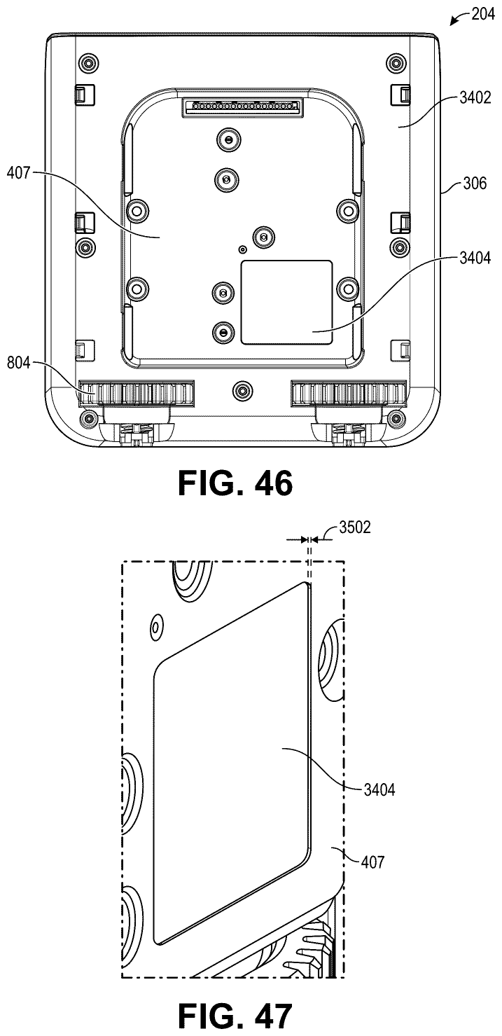

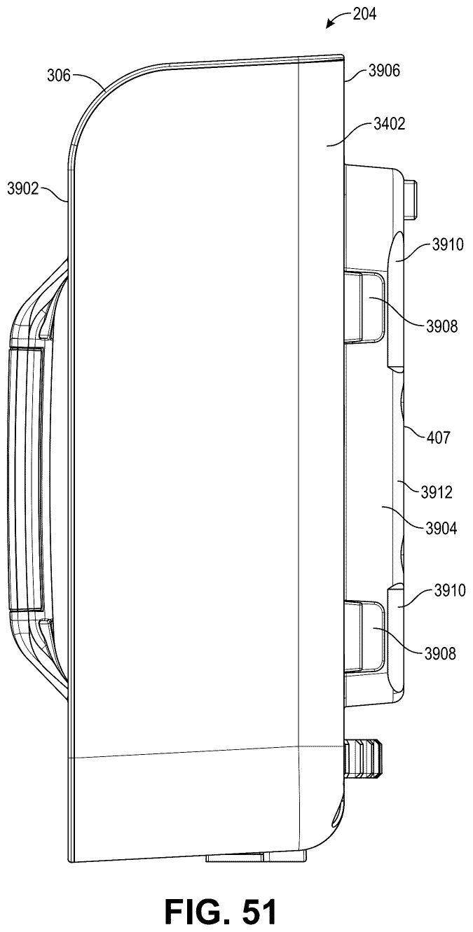

Referring to FIG. 46, a rear view of a cartridge shell of a fluid transfer cartridge is shown in accordance with an embodiment. The cartridge shell 306 of the fluid transfer cartridge 204 includes the cartridge cavity 402 defined between a front shell portion (FIG. 51) having the front face 404, and a rear shell portion 3402 having the rear face 407. In an embodiment, the rear shell portion 3402 includes a label recess 3404 (FIG. 47) in the rear face 407. The label recess 3404 can be a recessed cavity extending longitudinally forward into the rear face 407.

Referring to FIG. 47, a rear perspective view of a cartridge shell of a fluid transfer cartridge is shown in accordance with an embodiment. The label recess 3404 can be an indentation or void extending into the rear face 407 by a depth 3502.

A size of the label recess 3404 can correspond to a size of a label (not shown) received within the label recess 3404. For example, the label recess 3404 may have a profile including a height of about 1.3 inch and a width of about 1.5 inch. The dimensions may be slightly larger than the corresponding height and width of the label. Similarly, the depth 3502 of the label recess 3404 can correspond to a thickness of a label received within the label recess 3404.

The label may be mounted in the label recess 3404. The label can contain product information, and may have a predetermined thickness. For example, the thickness of the label can be a thickness of a substrate on which the product information is printed. The substrate may, for example, be 0.003-0.009 inch thick. In an embodiment, the depth 3502 of the label recess 3404 allows room for the label. More particularly, the label recess 3404 may be sized such that the label thickness is smaller than the depth 3502 of the label recess 3404. By way of example, the label recess 3404 can have a depth 3502 of 0.010-0.020 inch, e.g., 0.015 inch. Such depth 3502 can be greater than the substrate thickness, and thus, the label may not interfere with or contact the generator into which the cartridge shell 306 is loaded when the label is mounted within the label recess 3404. Accordingly, latches of the generator may align with corresponding recesses in the cartridge shell 306, as described below. The latches can therefore engage and lock the cartridge shell 306 to the generator.

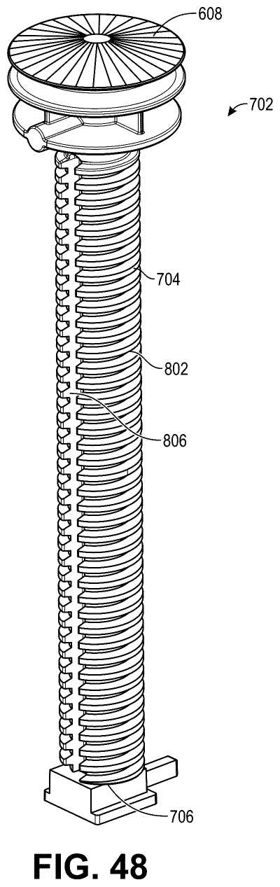

Referring to FIG. 48, a perspective view of a syringe piston of a fluid transfer cartridge is shown in accordance with an embodiment. As described above, the syringe piston 702 can be disposed within the syringe barrel 408. Similarly, the syringe barrel 408 can be disposed within the cartridge cavity 402 defined by the cartridge shell 306. The syringe piston 702 includes the stopper 608 and the shaft 704. The shaft 704 extends longitudinally from the stopper 608 to the shaft end 706. Furthermore, the shaft 704 includes the external thread 802, which as described above, can engage the internal thread 1109 (FIG. 50) of the gear 804. The gear 804 can be mounted on the cartridge shell 306 (FIG. 46).

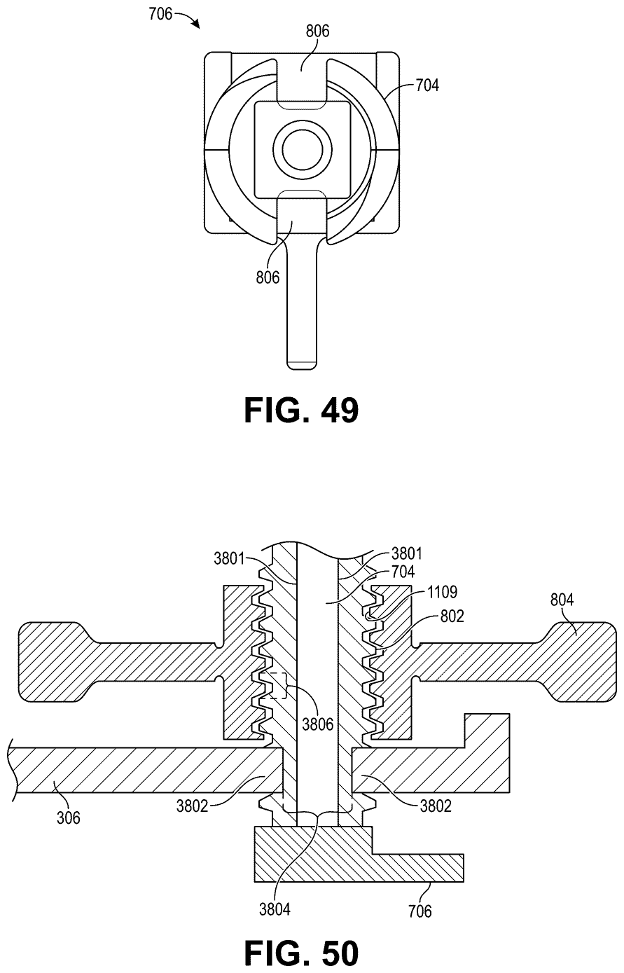

The piston notch 806 described above with respect to FIG. 20 is hidden behind the shaft 704 in FIG. 49. In an embodiment, the shaft 704 includes another piston notch 806. More particularly, the piston notch 806 illustrated in FIG. 49 can be diametrically opposite of the notch illustrated in FIG. 20.

Referring to FIG. 49, a cross-sectional view of a syringe piston is shown in accordance with an embodiment. The diametrically opposed notches 806 of the shaft 704 are visible in the end view of the cross-section. More particularly, the first piston notch 806 is shown at a six o'clock position and a second piston notch 806 is shown at a twelve o'clock position in the figure. Such notches 806 can be on opposite sides of the circular profile of the shaft 704. The cross-section is taken adjacent to the shaft end 706.

Referring to FIG. 50, a cross-sectional view of a fluid transfer cartridge is shown in accordance with an embodiment. The cross-section is taken transverse through the notches 806. Each piston notch 806 can extend into the shaft 704 to a respective notch base 3801. The notches 806 of the shaft 704 can provide anti-rotations features to limit or prevent rotation of the shaft 704 relative to the cartridge shell 306. More particularly, the notches 806 can restrict rotation of the shaft 704 relative to the gear 804, which moves when driven by the generator. Rotation of the shaft 704 relative to the cartridge shell 306 can be restricted by one or more stabilizing prongs 3802 that engage the notches 806 of the shaft 704. More particularly, the cartridge shell 306 can include several stabilizing prongs 3802 to insert into the notches 806 of the shaft 704. In an embodiment, the stabilizing prongs 3802 are separated by a gap 3804. The shaft 704 can extend longitudinally through the gap 3804 to the shaft end 706. More particularly, the shaft 704 can fit within the gap 3804 such that the stabilizing prongs 3802 are located in the notches of the shaft 704. When located in the notches 806, the stabilizing prongs 3802 can interfere with the walls surrounding the notches. The shaft 704 may therefore move vertically within the gap 3804 such that the stabilizing prongs 3802 move vertically within the notches 806, however, the stabilizing prongs 3802 and the shaft 704 may remain rotationally fixed relative to each other.

The gap may be a space filling a distance between prongs 3802. The prongs 3802 may be diametrically opposed, as shown in FIG. 50. In an embodiment, however, several prongs are not diametrically opposed. For example, the cartridge shell 306 can include two or more prongs 3802 that are disposed about a central shaft axis, without being diametrically opposed. There may be three prongs 3802 distributed about the central shaft axis such that the prongs are separated from each other by 120°. Four prongs 3802 may be separated from each other by 90°. Furthermore, distribution of the prongs 3802 may not be symmetrical. For example, two prongs can be separated by 60° and a third prong can be separated from the other two prongs by 150°. In any case, a line drawn transverse to the axial direction between adjacent prongs can extend through a distance between the prongs. The space filling the distance can be defined as the gap 3804. Accordingly, the gap 3804 may not extend through the central shaft axis but may nonetheless extend between prongs 3802.

One groove along the longitudinal axis of the screw, as described above, can provide anti-rotation. The addition of a second groove, e.g., the second piston notch 806, on an opposite side of the shaft 704 from the first groove can increase resistance to rotational movement. More particularly, several, e.g., two, stabilizing prongs 3802, rather than a single stabilizing prong 3802, can resist rotation of the shaft 704. Furthermore, reactive forces applied to the shaft 704 by the stabilizing prongs 3802 can be distributed over more surface area, which can reduce a likelihood of binding between the shaft 704 and the cartridge shell 306. Accordingly, rotational stabilization and longitudinal movement of the shaft 704 within the chassis of the cartridge shell 306 can be improved.

Binding between the shaft 704 and the cartridge shell 306 may occur when the stabilizing prong 3802 wedges between teeth of the external thread 802. To prevent such binding, the stabilizing prongs 3802 can have longitudinal widths that are greater than a pitch 3806 of the external thread 802. More particularly, a thickness of the stabilizing prong 3802 within the notch, measured in a longitudinal direction along the shaft length, can be greater than a distance in the longitudinal direction between adjacent teeth of the external thread 802. In an embodiment, the longitudinal widths of the stabilizing prongs 3802 are at least 1.5 times the pitch 3806 of the external thread 802. For example, the longitudinal width can measure twice the pitch 3806. Accordingly, the stabilizing prongs 3802 may be wide enough to always touch at least two teeth of the external thread 802, and thus, may be too big to fit between adjacent teeth of the external thread 802. The stabilizing prongs 3802 may therefore not bind between the teeth.

Referring to FIG. 51, a side view of a fluid transfer cartridge is shown in accordance with an embodiment. The cartridge shell 306 can define the cartridge cavity 402 between a front shell portion 3902 and the rear shell portion 3402. In an embodiment, the rear shell portion 3402 includes a sidewall 3904 extending longitudinally from a rear baseplate 3906 to the rear face 407. The rear shell portion 3402 can be inserted into a corresponding cavity of the generator to engage one or more latches of the generator. Accordingly, the fluid transfer cartridge 204 can include several latch keepers 3908 to receive the generator latches. The latch keepers 3908 can extend through the sidewall 3904. For example, the latch keepers 3908 can be cavities within which the generator latches can insert to engage the sidewall 3904 and prevent removal of the fluid transfer cartridge 204 from the generator.

Misalignment between the latch keepers 3908 and the generator latches can result in ineffective latching between the fluid transfer cartridge 204 and the generator. More particularly, it has been discovered that alignment between the latch keepers 3908 and the generator latches may be critical to effective function of the fluid transfer cartridge function. When the generator latches are not properly aligned with and/or guided into the latch keepers 3908, the latches may not engage and the fluid transfer cartridge 204 may not be secured within the generator. Accordingly, in an embodiment, the fluid transfer cartridge 204 includes several latch guides 3910 to widen an area for engagement with the generator latches, and to allow for an angled contact between the sets of latches.

The latch guides 3910 allow for the fluid transfer cartridge 204 to engage the generator latches at a wider range of entry angles. For example, the latch guides 3910 can be formed as rounded or beveled features along a rear edge 3912 between the sidewall 3904 and the rear face 407. The rear edge 3912 may be a sharp corner or a rounded or broken corner. In any case, the latch guides 3910 may have a larger radius than the rear edge 3912.

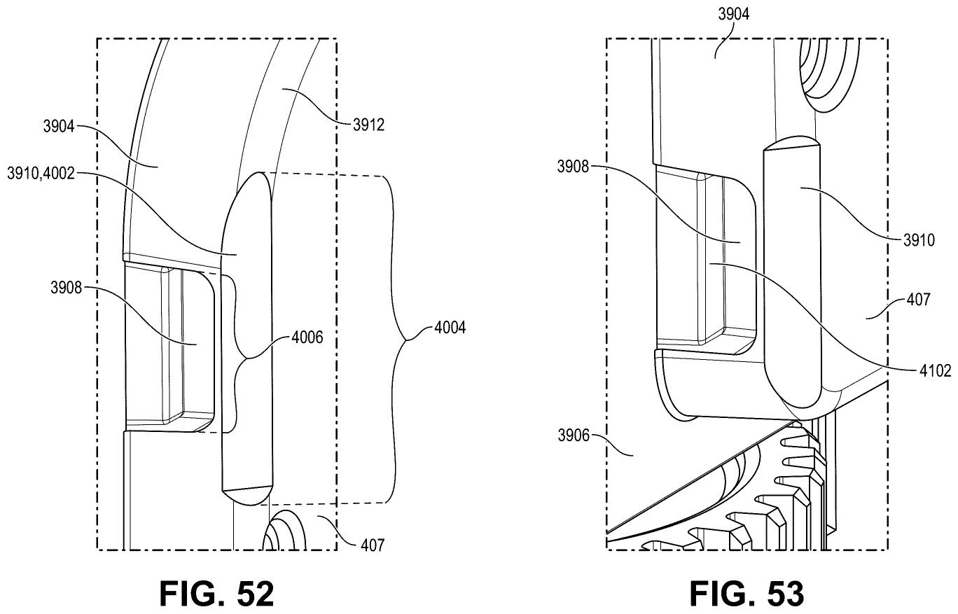

Referring to FIG. 52, a perspective view of a latch guide is shown in accordance with an embodiment. Each latch guide 3910 may include a bevel 4002 on the rear edge 3912. The bevel 4002 can be a chamfer that provides a gradual transition between the rear face 407 and the sidewall 3904. Each latch guide 3910 can be longitudinally aligned with a respective one of the latch keepers 3908. Accordingly, the latch guides 3910 can provide guidance of the generator latches over the rear shell portion 3402 into the corresponding latch keeper 3908. The latch guides 3910 can therefore increase the engagement range of the latch mechanism to improve performance of the fluid transfer cartridge 204.

In addition to guiding the generator latches longitudinally over the surface of the rear shell portion 3402 into the latch keeper 3908, the latch guides 3910 can be located to guide the generator latches vertically into the latch keeper 3908. For example, a length 4004 of the bevel 4002 of the latch guides 3910 may be greater than a width 4006 of the corresponding latch keeper 3908. When the generator latch is slightly higher or lower than the latch keeper 3908, the latch may nonetheless engage the latch guide 3910 and slide into place within the latch keeper 3908. Accordingly, making the latch guides 3910 wider than the latch keepers 3908 can facilitate secure engagement between the generator latches and the latch keepers 3908.

Referring to FIG. 53, a perspective view of a latch guide is shown in accordance with an embodiment. Each latch keeper 3908 can include a blind hole extending laterally into the sidewall 3904. More particularly, the guide keepers can have a depth defined by a thickness of a keeper wall 4102 extending around the latch keeper cavity. The thickness of the keeper wall 4102 and the depth of the keeper is sufficient to receive the generator latch. When the generator latch is engaged within the keeper, it can interfere with the keeper wall 4102 and therefore resist removal of the fluid transfer cartridge 204 from the generator.

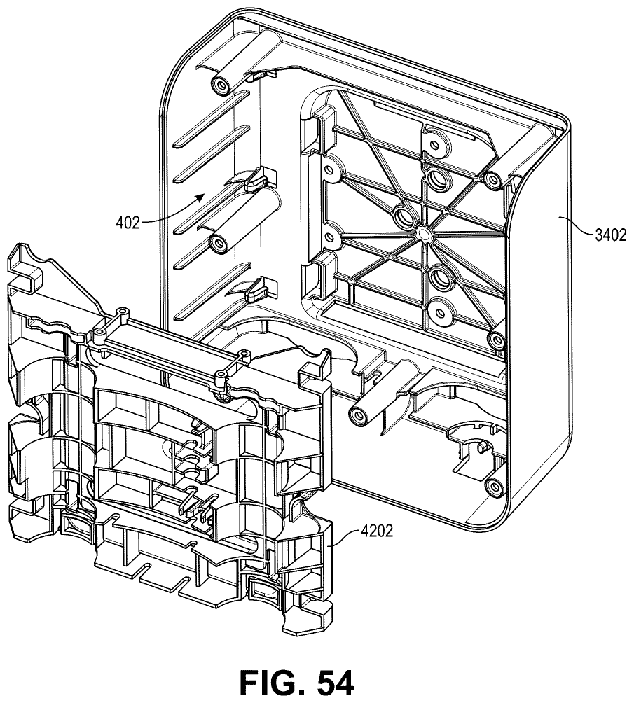

Referring to FIG. 54, a perspective exploded view of a fluid transfer cartridge is shown in accordance with an embodiment. The fluid transfer cartridge 204 can include a shell plate 4202 contained within the cartridge cavity 402. As described above, the cartridge cavity 402 can be defined between the front shell portion 3902 and the rear shell portion 3402 of the cartridge shell 306. The shell plate 4202 can be contained within the cartridge cavity 402 between the front shell portion 3902 and the rear shell portion 3402. More particularly, the shell plate 4202 can be inserted into an internal cavity of the rear shell portion 3402 and optionally attached thereto. Accordingly, rather than being a single piece design, the rear shell portion 3402 may include the outer shell having the sidewall 3904 and the rear face 407, and the shell plate 4202 is located within the outer shell in front of the rear face 407.

The rear shell portion 3402 can be realized by a multi-piece design. More particularly, the shell plate 4202 and the outer shell of the rear shell portion 3402 can be molded. Accordingly, per part costs and manufacturability of the fluid transfer cartridge 204 can be improved. Furthermore, the several pieces of the rear shell portion 3402 can interact to form the latch keepers 3908. The pieces can fit together such that the latch keepers 3908 are defined between the two pieces. The pieces can interlock to define latch keepers 3908, and the interlocking structure may provide a stable structure. More particularly, the interlocking parts can fit together tightly to provide a stable system that engages securely with the generator latches.

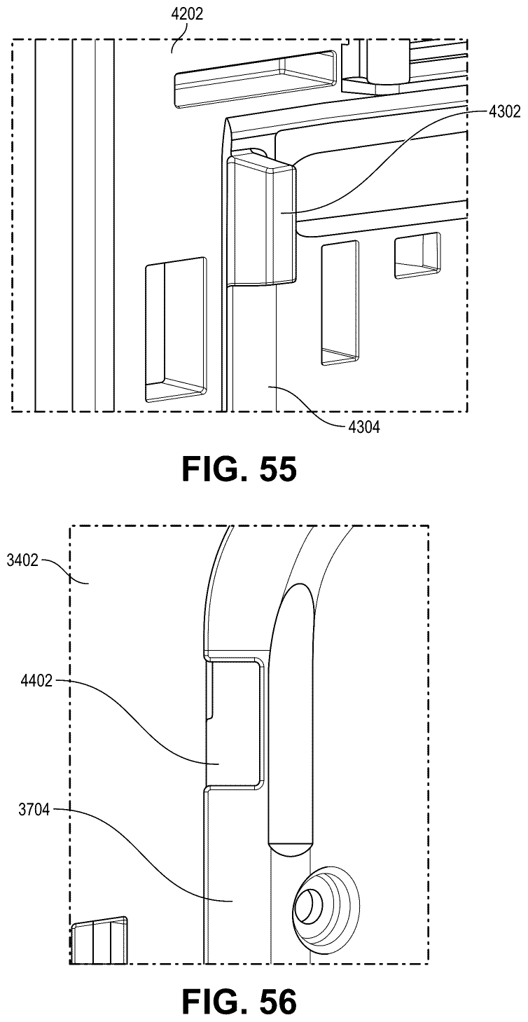

Referring to FIG. 55, a perspective view of a shell plate of a fluid transfer cartridge is shown in accordance with an embodiment. The shell plate 4202 can include several lateral projections 4302 extending from a plate surface 4304. The plate surface 4304 can be a surface that the lateral projections 4302 are located on. The lateral projections 4302, for example, can be rectangular bosses that extend outward from the plate surface 4304.

Referring to FIG. 56, a perspective view of a cartridge shell of a fluid transfer cartridge is shown in accordance with an embodiment. The cartridge shell 306, e.g., the rear shell portion 3402 can include several latch holes 4402. The latch holes 4402 can extend through the sidewall 3904 of the rear shell portion 3402. The latch holes 4402 can be through-holes that extend from an outward surface of the sidewall 3904 into the interior of the cartridge shell 306 that contains the shell plate 4202.

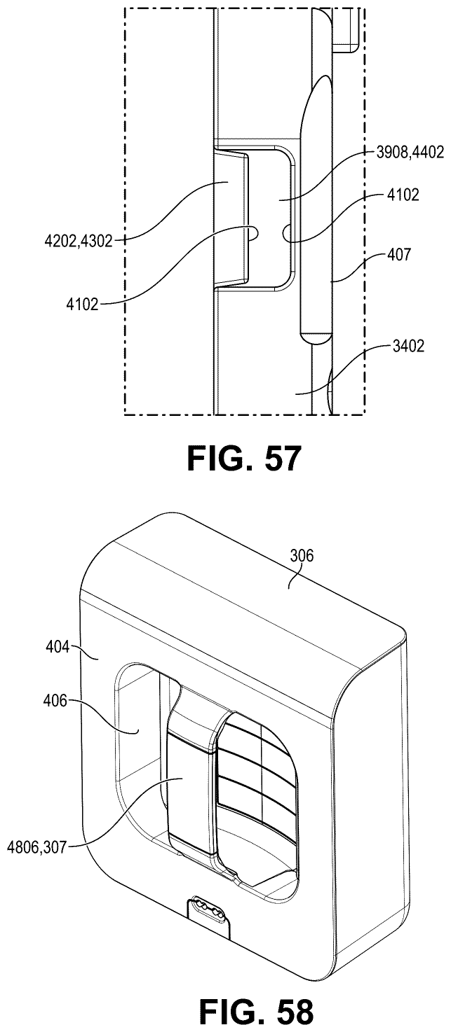

Referring to FIG. 57, a side view of a latch keeper is shown in accordance with an embodiment. When the shell plate 4202 is inserted into the cartridge shell 306, the lateral projections 4302 of the shell plate 4202 can insert into corresponding latch holes 4402 to form the latch keepers 3908. More particularly, the lateral projections 4302 can fill a portion of the latch holes 4402, and the remaining recess (the unfilled portion) can be the latch keeper 3908. The latch keeper 3908 may be defined between the keeper walls 4102 which face longitudinally toward each other across the latch keeper recess. A portion of the keeper wall 4102 can be on the lateral projection 4302, and a portion of the keeper wall 4102 can be on the sidewall 3904. The latch keepers 3908 can therefore be defined between the lateral projections 4302 and the sidewall 3904 or the rear face 407. Accordingly, when the generator latches engage the latch keeper 3908, the latches can be located between the rear face 407 and a lateral projections 4302.

Referring to FIG. 58, a perspective view of a cartridge shell is shown in accordance with an embodiment. The cartridge shell 306 of the fluid transfer cartridge 204 can include a handle 307, as described above. The handle 307 can extend from the front face 404 of the cartridge shell 306 over the opening, e.g., the window 406. The handle 307 allows an operator to grip and physically control the fluid transfer cartridge 204. For example, the operator can grab the handle 307 to engage the fluid transfer cartridge 204 into the generator and remove the cartridge from the generator.

The cartridge shell 306 of the fluid transfer cartridge 204 may be injection molded. Injection molding requires molding gates that can leave gate marks, such as indentations and/or protrusions. Furthermore, parts may be ribbed to facilitate the molding process. Such protrusions and ribbing can compromise the grip of the operator. Furthermore, such surface features may snag on a glove of the operator. Accordingly, there may be a need to improve the handle interface.

In an embodiment, the handle 307 is formed such that a front surface 4806 of the handle 307 is entirely smooth. More particularly, the front surface 4806 can be flat and smooth without a molding gate mark. The smooth front surface 4806 is shown in FIG. 58, and can create a seamless aesthetic.

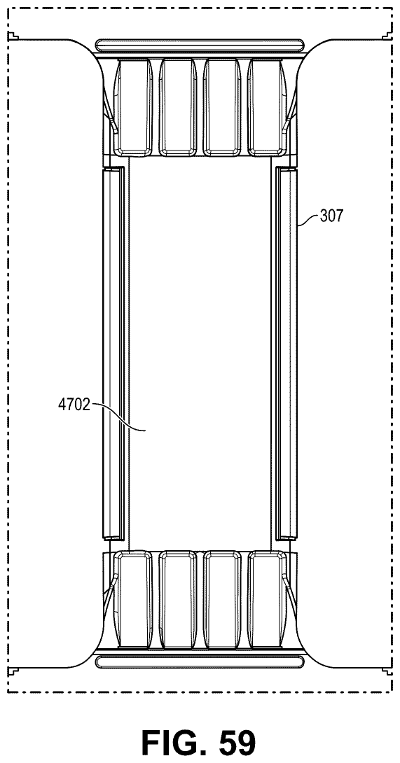

Referring to FIG. 59, a rear view of a handle of a cartridge shell is shown in accordance with an embodiment. Similar to the front surface 4806 of the handle 307, the handle 307 may include a rear surface 4702. The rear surface 4702, like the front surface 4806, may be entirely smooth. For example, the rear surface 4702 may have no molding gate marks, e.g., protrusions or snags.

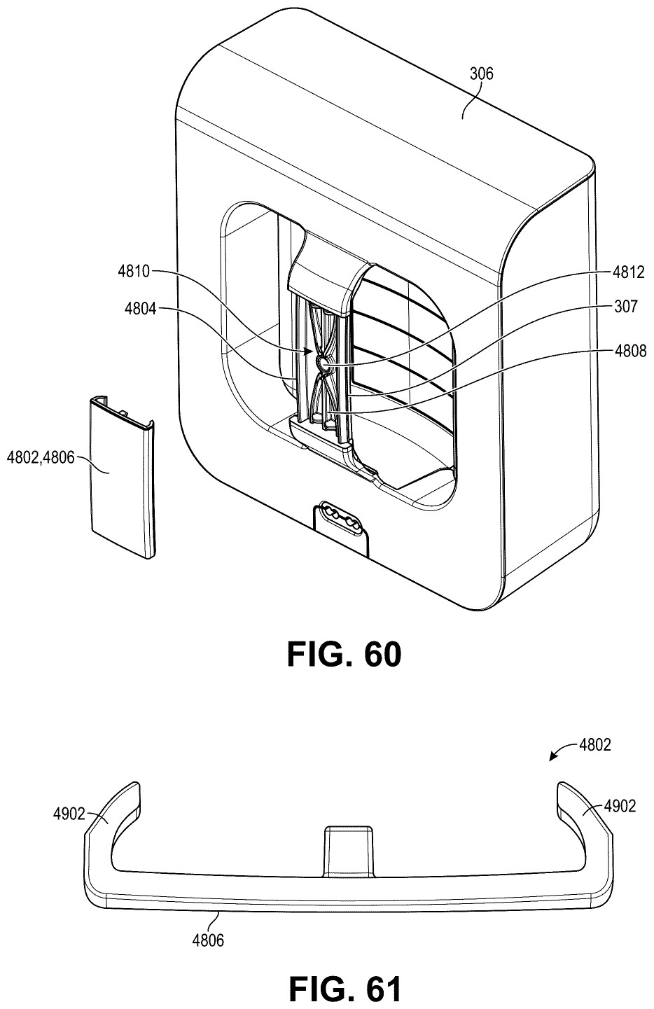

Referring to FIG. 60, a perspective exploded view of a fluid transfer cartridge is shown in accordance with an embodiment. The smooth outer surface of the handle 307 may be achieved using a multipart design. More particularly, the handle 307 can include a detachable front plate 4802 that attaches to a rear handle portion 4804. The detachable front plate 4802 can be mounted on the rear handle portion 4804, and can have the front surface 4806 that is entirely smooth. More particularly, the front surface 4806 can lack protrusions, indentations, or other features resulting from molding gates. Similarly, as described above, the rear handle portion 4804 can have the rear surface 4702 that is entirely smooth.

Removal of the detachable front plate 4802 from the rear handle portion 4804 can expose an anterior surface 4808 of the rear handle portion 4804. The anterior surface 4808 can have ribbing, which is an artifact of the molding process used to form the front shell portion 3902. The ribbing meets in a middle 4810 of the anterior surface 4808. In an embodiment, the anterior surface 4808 has a molding gate 4812. The molding gate 4812 can be located at the middle 4810 of the anterior surface 4808. When the detachable front plate 4802 is mounted on the rear handle portion 4804, the plate can cover the molding gate 4812. When removed, however, the molding gate 4812 can be exposed. Accordingly, when attached to the rear handle portion 4804, the detachable front plate 4802 can contain the molding artifacts between the smooth front surface 4806 and the smooth rear surface 4702 of the handle 307.

Referring to FIG. 61, an end view of a handle of a fluid transfer cartridge is shown in accordance with an embodiment. The end view reveals a profile of the front surface 4806. In profile, the front surface 4806 is continuously smooth. For example, the profile can be flat or curvilinear. More particularly, the front surface 4806 does not have any abrupt edges or protrusions that can snag on a glove. The detachable front plate 4802 can include several clips 4902 that extend rearward from the front surface 4806. The clips 4902 can curve inward toward a midplane of the detachable front plate 4802. The clips 4902 can therefore wrap around and snap on to the rear handle portion 4804 to secure the detachable front plate 4802 to the cartridge shell 306.

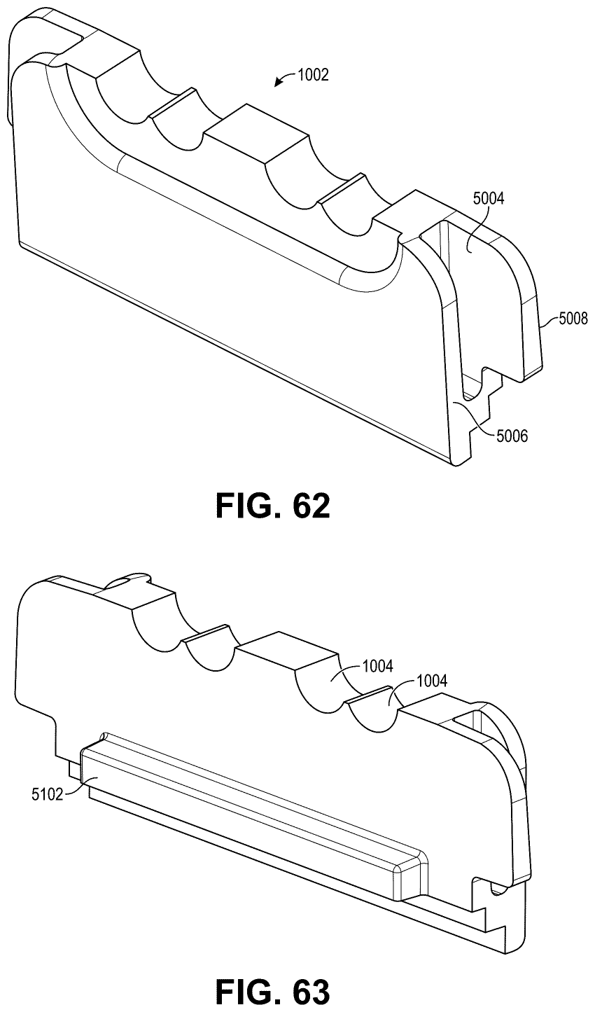

Referring to FIG. 62, a front perspective view of a conduit routing plate is shown in accordance with an embodiment. The fluid transfer cartridge 204 can include the conduit routing plate 1002. The conduit routing plate 1002 may be used to cover a conduit port in the front face 404 of the cartridge shell 306. The conduit routing plate 1002 can be shaped to securely engage one or more features of the cartridge shell 306. For example, the conduit routing plate 1002 can include a conduit slot 5004 formed between a front ledge 5006 and a rear ledge 5008. As described below, the conduit slot 5004 can receive a feature of the cartridge shell 306 to secure the conduit routing plate 1002 and lock it into place relative to the cartridge shell 306.

In an embodiment, the conduit routing plate 1002 is formed from a material having high strength, high stiffness, high heat resistance, and high impact resistance. Such properties may exist even at low temperatures. By way of example, the conduit routing plate 1002 may be formed from a poly carbonate/acrylonitrile butadiene styrene (PC-ABS) material. Such material can maintain dimensional stability over time. Accordingly, conduit routing plate 1002 can be robust and durable.

Referring to FIG. 63, a rear perspective view of a conduit routing plate is shown in accordance with an embodiment. The conduit routing plate 1002 can include one or more notch 1006, as described above. The notches 1006 can be grooves that mate with protrusions on the cartridge shell 306 to form ports through which the fluid conduit 206 may be routed. The conduit routing plate 1002 may include additional features that mate with corresponding features of the cartridge shell 306. For example, a rear ledge 5102 may extend rearward from a body of the conduit routing plate 1002. The rear ledge 5102 may engage a corresponding portion of the cartridge shell 306 to secure the conduit routing plate 1002 to the cartridge shell 306. For example, the rear ledge 5102 can insert into a corresponding slot, or rest on a corresponding ledge, of rear shell portion 3402.

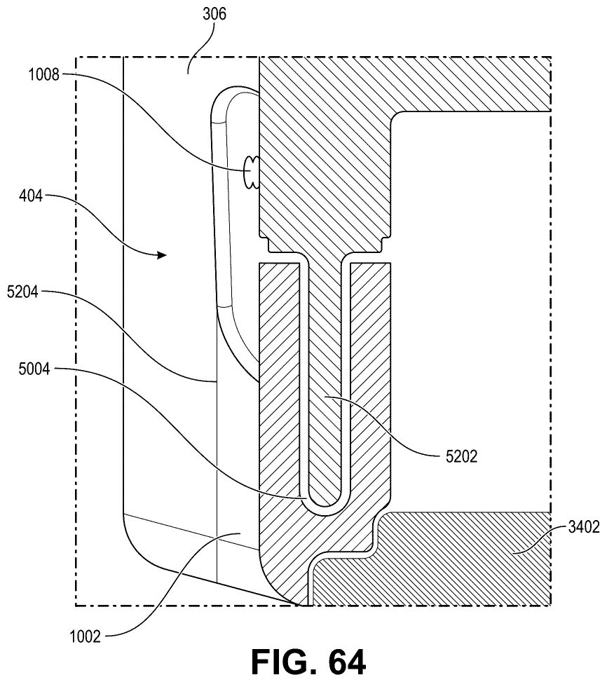

Referring to FIG. 64, a cross-sectional perspective view of a conduit routing plate mounted on a cartridge shell is shown in accordance with an embodiment. The cross-sectional view illustrates the interlocking features of the cartridge shell 306 and the conduit routing plate 1002. More particularly, the front face 404 of the cartridge shell 306 can include a tab 5202 that extends into the conduit port. The conduit port can be a window defined in a front surface of front face 404, e.g., by an edge 5204. Note that the conduit port is filled by the conduit routing plate 1002 in FIG. 64, but would be a through hole in the front face 404 when the conduit routing plate 1002 is removed. Nonetheless, the tab 5202 can be located in the conduit slot 5004 of the conduit routing plate 1002 when the conduit routing plate 1002 is engaged with the front face 404. The conduit routing plate 1002 can engage the front face 404 along the edge 5204 that defines the conduit port. The conduit routing plate 1002 can be snapped into place, which can improve manufacturability. When the conduit routing plate 1002 is so engaged, the protruding features, such as tab 5202, of the front shell can lock the conduit routing plate 1002 in place.

The interlocked cartridge shell 306 and conduit routing plate 1002 can define the conduit routing ports 1008. More particularly, the notches 1006 in the front face 404 of the cartridge shell 306 and the notches 1006 in the conduit routing plate 1002, all of which can be along the edge 5204, can combine to form the conduit routing port 1008. The conduit routing ports 1008 provide ports through which the fluid conduit 206 may be routed.

Referring to FIG. 65, a perspective view of a piston of a cartridge manifold is shown in accordance with an embodiment. FIG. 66 shows a cross-sectional view of the piston of the cartridge manifold in accordance with an embodiment. The figures, FIGS. 65-66, are described in combination below.

The valves used to open and close the fluid ports 1706 can include the pistons 1610. More particularly, the pistons 1610 can interact with the fluid transfer plate 1602 to seal and unseal the fluid ports 1706. As described above, the piston 1610 can include an end seal 1902. The piston 1610 can be placed in an open position in which the end seal 1902 unseals (does not occlude) a corresponding fluid port 1706 to allow cooling fluid 603 to pass through the fluid port 1706. The piston 1610 can be moved from the open position to a closed position in which the end seal 1902 seals (occludes) the corresponding fluid port 1706 to stop the cooling fluid 603 from passing through the fluid port 1706. Accordingly, the piston 1610 acts as a valve by covering or uncovering the fluid port 1706 to control fluid flow therethrough.

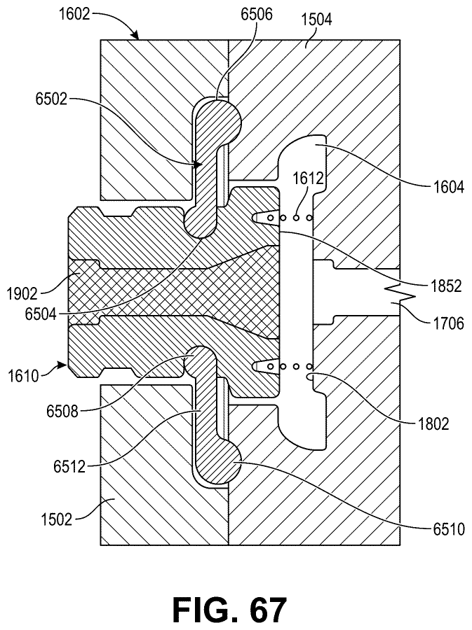

As described above, the piston 1610 can be spring-loaded. The piston 1610 may include the spring groove 1904. The spring groove 1904 can include an annular groove sized and shaped to receive a proximal end of the spring 1612 (FIG. 67). The spring 1612 can be a helical compression spring 1612. A distal end of the spring 1612 can be, optionally, similarly engaged to a corresponding spring groove at the valve seat. The spring grooves 1904 can stabilize the spring 1612, and allow the spring 1612 to act against both the rear plate surface 1802 and the piston 1610. Accordingly, the spring 1612 can bias the piston 1610 outward to maintain the piston 1610 in a normally open position in which the end seal 1902 is offset from the rear plate surface 1802 to allow fluid flow through the fluid port 1706.

The piston 1610 can include a diaphragm seal 1906 to form a seal between the piston 1610 and one or more of the manifold plates. The diaphragm seal 1906 can have an annular disc structure, including an inner perimeter 6504 and an outer perimeter 6506. In an embodiment, the diaphragm seal 1906 includes an inner lip 6508 (FIG. 66) having the inner perimeter 6504 and an outer lip 6510 having the outer perimeter 6506. The lips can have substantially round cross-sectional profiles, similar to O-rings, such as those of channel seals 1710. The lips 6508, 6510 can therefore be located against respective surfaces to form a seal. For example, the inner lip 6508 can fit within a side groove of the piston 1610 to seal against a surface of the side groove. Similarly, the outer lip 6510 can seal against surfaces of the fluid transfer plate 1602. For example, the outer lip 6510 can be in contact with and clamped between adjacent plates, e.g., the fore plate 1502 and the aft plate 1504, to form a seal against the fluid transfer plate 1602 (FIG. 67).

The diaphragm seal 6502 can include a seal flange 6512 extending radially between the lips. The seal flange 6512 can include a thin annular membrane. More particularly, the seal flange 6512 can be disc-shaped and have a thickness that is less than a cross-sectional dimension of the inner lip 6508 or the outer lip 6510. The seal flange 6512 can extend radially between the lips 6508, 6510 to constrain the lips relative to each other. More particularly, the lips can be at the outer edges of the flange and can move relative to each other when the flange flexes. The diaphragm seal 1906 can be formed from an elastomeric material, such as silicone. Accordingly, the lips and the flange form portions of a flexible seal that can seal against and provide relative movement between the piston 1610 and the fluid transfer plate 1602.

Referring to FIG. 67, a sectional view, taken about line A-A of FIG. 30, of a piston of a cartridge manifold in an open position is shown in accordance with an embodiment. The piston 1610 can be a free floating piston having a diaphragm seal 6502 to radially seal against a side surface of the piston and the aft plate 1504, as described above. Furthermore, the end seal 1902 can face the fluid port 1706 in the fluid transfer plate 1602. In the open position, the spring 1612 can maintain the end seal 1902 spaced apart from the fluid port 1706. Furthermore, the fluid pressure within the fluid channels in front of the end face 1852 can press against the end face 1852, biasing the piston 1610 to the open position. Accordingly, cooling fluid 603 may flow through the front fluid channel 1604 and the fluid port 1706 into the rear fluid channel 1804.

As described above, a solenoid 2202 can be actuated to press the piston 1610 forward. The solenoid 2202 force can overcome the spring 1612 and fluid pressure acting against the piston 1610 in an opposite direction to cause the piston 1610 to move to the closed position (not shown, but similar to FIG. 34). In the closed position, the end seal 1902 obstructs the path of fluid flow through the fluid port 1706. More particularly, the cooling fluid 603 is stopped from flowing through the fluid port 1706 to or from the front fluid channel 1604.

It will be appreciated that, during actuation between the open position and the closed position, the seal formed by the seal flange 6512 is maintained without the requirement of the lips sliding over an external surface. More particularly, rather than side seals 1906 sliding against an adjacent surface, the lips can remain engaged within respective grooves and the flange can flex to permit relative movement of the adjacent surfaces. Such action, i.e., flexing rather than sliding, can be advantageous because the diaphragm seal 6502 can maintain placement and function of the seal that controls motion of the piston axially relative the fluid transfer plate, while also eliminating the need for lubrication of sliding seals and/or the concomitant friction that can result in seal failure.

Embodiments of a treatment system are described above. More particularly, embodiments of the treatment system are described, either explicitly or implicitly. The following paragraphs summarize some of the described embodiments. More particularly, embodiments are described in the following enumerated examples.

Example 1. A method, comprising: delivering, by a fluid transfer unit of a tissue treatment system, inflation fluid at an inflation pressure to a balloon; injecting, by a contrast injector of the tissue treatment system, contrast medium at an injection pressure into a vessel containing the balloon; determining one or more of the inflation pressure or the injection pressure; and stopping, based on one or more of the inflation pressure or the injection pressure, injection of the contrast medium.

Example 2. The method of claim 1, wherein injection of the contrast medium is stopped based on the injection pressure being equal to an injection pressure limit.

Example 3. The method of claim 1, wherein determining the injection pressure is based on a motor work value of a drive motor of the contrast injector.

Example 4. The method of claim 1, wherein injection of the contrast medium is stopped based on the inflation pressure being equal to an inflation pressure limit.

Example 5. The method of claim 1, wherein injection of the contrast medium is stopped based on the injection pressure being equal to the inflation pressure.

Example 6. The method of claim 1 further comprising: stopping injection of the contrast medium in response to a target volume of contrast medium being delivered into the vessel; and displaying a prompt requesting confirmation of whether the vessel is occluded.

Example 7. The method of claim 1 further comprising: receiving a user input confirming that the vessel is occluded; and displaying, in response to the user input confirming that the vessel is occluded, an initiate control element selectable to initiate delivery, by an energy delivery unit of the tissue treatment system, energy to an ultrasound transducer contained within the balloon to emit acoustic energy through the inflation fluid to an ablation target.

Example 8. The method of claim 7 further comprising: receiving a user input confirming that the vessel is not occluded; and displaying, in response to the user input confirming that the vessel is not occluded, a resume control element selectable to resume contrast injection.

Example 9. The method of claim 1 further comprising stopping injection of the contrast medium in response to a predetermined maximum volume of contrast medium being delivered into the vessel.

Example 10. The method of claim 1 further comprising determining whether the contrast injector is in electrical communication with an energy delivery unit of the tissue treatment system.

Example 11. A non-transitory computer-readable medium storing instructions which, when executed by one or more processors of a tissue treatment system, cause the tissue treatment system to perform a method comprising: delivering, by a fluid transfer unit of the tissue treatment system, inflation fluid at an inflation pressure to a balloon; injecting, by a contrast injector of the tissue treatment system, contrast medium at an injection pressure into a vessel containing the balloon; determining, by the one or more processors, one or more of the inflation pressure or the injection pressure; and stopping, based on one or more of the inflation pressure or the injection pressure, injection of the contrast medium by the contrast injector.

Example 12. The non-transitory computer-readable medium of claim 11, wherein injection of the contrast medium is stopped based on the injection pressure being determined to be equal to an injection pressure limit.

Example 13. The non-transitory computer-readable medium of claim 11, wherein determining the injection pressure is based on a motor work value of a drive motor of the contrast injector.

Example 14. The non-transitory computer-readable medium of claim 11, wherein injection of the contrast medium is stopped based on the inflation pressure being determined to be equal to an inflation pressure limit.

Example 15. The non-transitory computer-readable medium of claim 11, wherein injection of the contrast medium is stopped based on the injection pressure being equal to the inflation pressure.

Example 16. The non-transitory computer-readable medium of claim 11, wherein the method further comprises: stopping injection of the contrast medium in response to a target volume of contrast medium being delivered into the vessel; and displaying a prompt requesting confirmation of whether the vessel is occluded.

Example 17. The non-transitory computer-readable medium of claim 16, wherein the method further comprises: receiving a user input confirming that the vessel is occluded; and displaying, in response to the user input confirming that the vessel is occluded, an initiate control element selectable to initiate delivery, by an energy delivery unit of the tissue treatment system, energy to an ultrasound transducer contained within the balloon to emit acoustic energy through the inflation fluid to an ablation target.

Example 18. The non-transitory computer-readable medium of claim 16, wherein the method further comprises: receiving a user input confirming that the vessel is not occluded; and displaying, in response to the user input confirming that the vessel is not occluded, a resume control element selectable to resume contrast injection.

Example 19. The non-transitory computer-readable medium of claim 11, wherein the method further comprises stopping injection of the contrast medium in response to a predetermined maximum volume of contrast medium being delivered into the vessel.

Example 20. A tissue treatment system, comprising: a fluid transfer unit to deliver an inflation fluid at an inflation pressure to a balloon; a contrast injector to inject contrast medium at an injection pressure into a vessel containing the balloon; a memory storing instructions; and one or more processors configured to execute the instructions to determine one or more of the inflation pressure or the injection pressure, and cause the contrast injector to stop injection of the contrast medium based on one or more of the inflation pressure or the injection pressure.

Example 21. The tissue treatment system of claim 20, wherein the contrast injector includes a housing to receive a syringe containing the contrast medium, and wherein the housing includes a pivotable cap having a slot sized to receive a plunger flange of the syringe.

Example 22. The tissue treatment system of claim 21, wherein the slot is sized to receive a second plunger flange of a second syringe sized differently than the plunger flange of the syringe.

Example 23. The tissue treatment system of claim 20 further comprising: a tissue treatment catheter, an ultrasound transducer contained within the balloon; and an energy delivery unit to energize the ultrasound transducer to emit acoustic energy through the inflation fluid to an ablation target.

Example 24. The tissue treatment system of claim 20 further comprising: a contrast reservoir to store contrast medium, the contrast reservoir located in a fluid transfer cartridge.

Example 25. The tissue treatment system of claim 24, wherein the contrast reservoir is located proximate to a first syringe barrel.

Example 26. The tissue treatment system of claim 25, wherein the contrast reservoir is located between the first syringe barrel and a second syringe barrel.

Example 27. The tissue treatment system of claim 24, wherein the contrast reservoir is located behind a handle front plate.

Example 28. A fluid transfer cartridge, comprising: a cartridge shell defining a cartridge cavity, wherein the cartridge shell includes one or more stabilizing prongs; a syringe barrel disposed within the cartridge cavity; and a syringe piston disposed within the syringe barrel, wherein the syringe piston includes a stopper and a shaft extending longitudinally from the stopper to a shaft end, and wherein the shaft includes a plurality of piston notches each of which receives at least one of the one or more stabilizing prongs.

Example 29. The fluid transfer cartridge of claim 28, wherein two of the stabilizing prongs face each other and the shaft extends through a gap separating the two stabilizing prongs.

Example 30. The fluid transfer cartridge of claim 29, wherein the shaft includes an external thread, and further comprising a gear mounted on the cartridge shell, wherein the gear includes an internal thread engaging the external thread.

Example 31. The fluid transfer cartridge of claim 30, wherein the one or more stabilizing prongs have longitudinal widths greater than a pitch of the external thread.

Example 32. The fluid transfer cartridge of claim 31, wherein the longitudinal widths of the one or more stabilizing prongs are at least 1.5 times the pitch of the external thread.

Example 33. A fluid transfer cartridge, comprising: a cartridge shell defining a cartridge cavity, wherein the cartridge shell includes a plurality of stabilizing prongs separated by a gap; a syringe barrel disposed within the cartridge cavity; and a syringe piston disposed within the syringe barrel, wherein the syringe piston includes a stopper and a shaft extending longitudinally from the stopper through the gap to a shaft end.

Example 34. The fluid transfer cartridge of claim 33, wherein the plurality of stabilizing prongs are located in a plurality of piston notches of the shaft.

Example 35. The fluid transfer cartridge of claim 34, wherein the shaft includes an external thread, and further comprising a gear mounted on the cartridge shell, wherein the gear includes an internal thread engaging the external thread.

Example 36. The fluid transfer cartridge of claim 35, wherein the plurality of stabilizing prongs have longitudinal widths greater than a pitch of the external thread.

Example 37. The fluid transfer cartridge of claim 36, wherein the longitudinal widths of the plurality of stabilizing prongs are at least 1.5 times the pitch of the external thread.

Example 38. A fluid transfer cartridge, comprising: a cartridge shell defining a cartridge cavity between a front shell portion and a rear shell portion, wherein the rear shell portion includes a sidewall extending longitudinally from a rear base plate to a rear face, wherein a plurality of latch keepers extend through the sidewall, and wherein a plurality of latch guides are formed along an edge between the sidewall and the rear face.

Example 39. The fluid transfer cartridge of claim 38, wherein each latch guide of the plurality of latch guides is longitudinally aligned with a respective one of the plurality of latch keepers.

Example 40. The fluid transfer cartridge of claim 38, wherein each latch guide of the plurality of latch guides includes a bevel on the edge.

Example 41. The fluid transfer cartridge of claim 40, wherein a length of the bevel is greater than a width of the respective one of the plurality of latch keepers.

Example 42. A fluid transfer cartridge, comprising: a cartridge shell defining a cartridge cavity between a front shell portion and a rear shell portion, wherein the rear shell portion includes a label recess in a rear face, and wherein the label recess has a depth of 0.01 to 0.02 inch.

Example 43. The fluid transfer cartridge of claim 42, wherein the depth is 0.015 inch.

Example 44. The fluid transfer cartridge of claim 42, further comprising a label mounted in the label recess, wherein the label has a thickness that is smaller than the depth of the label recess.

Example 45. A fluid transfer cartridge, comprising: a cartridge shell defining a cartridge cavity between a front shell portion and a rear shell portion, wherein the rear shell portion includes a sidewall extending longitudinally from a rear base plate to a rear face, and wherein a plurality of latch holes extend through the sidewall; and a shell plate contained within the cartridge cavity between the front shell portion and the rear shell portion, wherein the shell plate includes a plurality of lateral projections extending into the plurality of latch holes such that a plurality of latch keepers are defined between the plurality of lateral projections and the rear face.

Example 46. A fluid transfer cartridge, comprising: a cartridge shell defining a cartridge cavity between a front face and a rear face, wherein the cartridge shell includes a handle extending from the front face over an opening, and wherein the handle includes a detachable front plate.

Example 47. The fluid transfer cartridge of claim 46, wherein the handle includes the detachable front plate mounted on a rear handle portion, and wherein a front surface of the detachable front plate is entirely smooth.

Example 48. The fluid transfer cartridge of claim 47, wherein a rear surface of the rear handle portion is entirely smooth.

Example 49. The fluid transfer cartridge of claim 48, wherein an anterior surface of the rear handle portion has a molding gate, and wherein the detachable front plate covers the molding gate.

Example 50. The fluid transfer cartridge of claim 49, wherein the molding gate is located at a middle of the anterior surface.

Example 51. A fluid transfer cartridge, comprising: a cartridge shell defining a cartridge cavity between a front face and a rear face, wherein the front face includes a tab extending into a conduit port defined by an edge; and a conduit routing plate engaging the front face along the edge, wherein the tab is located in a conduit slot of the conduit routing plate.

Example 52. The fluid transfer cartridge of claim 51, wherein the front face and the conduit routing plate include respective notches at the edge, and wherein the respective notches combine to form a conduit routing port through which a fluid conduit is routed.

In the foregoing specification, the present disclosure has been described with reference to specific exemplary embodiments thereof. It will be evident that various modifications may be made thereto without departing from the broader spirit and scope of the present disclosure as set forth in the following claims. The specification and drawings are, accordingly, to be regarded in an illustrative sense rather than a restrictive sense.

What is claimed is:

1. A method, comprising:
delivering, by a fluid transfer unit of a tissue treatment system, inflation fluid at an inflation pressure to a balloon;
injecting, by a contrast injector of the tissue treatment system, contrast medium at an injection pressure into a vessel containing the balloon;
determining one or more of the inflation pressure or the injection pressure; and
stopping, based on one or more of the inflation pressure or the injection pressure, injection of the contrast medium.

2. The method of claim 1, wherein injection of the contrast medium is stopped based on the injection pressure being equal to an injection pressure limit.

3. The method of claim 1, wherein determining the injection pressure is based on a motor work value of a drive motor of the contrast injector.

4. The method of claim 1, wherein injection of the contrast medium is stopped based on the inflation pressure being equal to an inflation pressure limit.

5. The method of claim 1, wherein injection of the contrast medium is stopped based on the injection pressure being equal to the inflation pressure.

6. The method of claim 1 further comprising:
stopping injection of the contrast medium in response to a target volume of contrast medium being delivered into the vessel; and
displaying a prompt requesting confirmation of whether the vessel is occluded.

7. The method of claim 1 further comprising:
receiving a user input confirming that the vessel is occluded; and
displaying, in response to the user input confirming that the vessel is occluded, an initiate control element selectable to initiate delivery, by an energy delivery unit of the tissue treatment system, energy to an ultrasound transducer contained within the balloon to emit acoustic energy through the inflation fluid to an ablation target.

8. The method of claim 7 further comprising:
receiving a user input confirming that the vessel is not occluded; and
displaying, in response to the user input confirming that the vessel is not occluded, a resume control element selectable to resume contrast injection.

9. The method of claim 1 further comprising stopping injection of the contrast medium in response to a predetermined maximum volume of contrast medium being delivered into the vessel.

10. The method of claim 1 further comprising determining whether the contrast injector is in electrical communication with an energy delivery unit of the tissue treatment system.

11. A non-transitory computer-readable medium storing instructions which, when executed by one or more processors of a tissue treatment system, cause the tissue treatment system to perform a method comprising:
delivering, by a fluid transfer unit of the tissue treatment system, inflation fluid at an inflation pressure to a balloon;
injecting, by a contrast injector of the tissue treatment system, contrast medium at an injection pressure into a vessel containing the balloon;
determining, by the one or more processors, one or more of the inflation pressure or the injection pressure; and
stopping, based on one or more of the inflation pressure or the injection pressure, injection of the contrast medium by the contrast injector.

12. The non-transitory computer-readable medium of claim 11, wherein injection of the contrast medium is stopped based on the injection pressure being determined to be equal to an injection pressure limit.

13. The non-transitory computer-readable medium of claim 11, wherein determining the injection pressure is based on a motor work value of a drive motor of the contrast injector.

14. The non-transitory computer-readable medium of claim 11, wherein injection of the contrast medium is stopped based on the inflation pressure being determined to be equal to an inflation pressure limit.

15. The non-transitory computer-readable medium of claim 11, wherein injection of the contrast medium is stopped based on the injection pressure being equal to the inflation pressure.

16. The non-transitory computer-readable medium of claim 11, wherein the method further comprises:
stopping injection of the contrast medium in response to a target volume of contrast medium being delivered into the vessel; and
displaying a prompt requesting confirmation of whether the vessel is occluded.

17. The non-transitory computer-readable medium of claim 16, wherein the method further comprises:
receiving a user input confirming that the vessel is occluded; and
displaying, in response to the user input confirming that the vessel is occluded, an initiate control element selectable to initiate delivery, by an energy delivery unit of the tissue treatment system, energy to an ultrasound transducer contained within the balloon to emit acoustic energy through the inflation fluid to an ablation target.

18. The non-transitory computer-readable medium of claim 16, wherein the method further comprises:
receiving a user input confirming that the vessel is not occluded; and
displaying, in response to the user input confirming that the vessel is not occluded, a resume control element selectable to resume contrast injection.

19. The non-transitory computer-readable medium of claim 11, wherein the method further comprises stopping injection of the contrast medium in response to a predetermined maximum volume of contrast medium being delivered into the vessel.

20. A tissue treatment system, comprising:
a fluid transfer unit to deliver an inflation fluid at an inflation pressure to a balloon;
a contrast injector to inject contrast medium at an injection pressure into a vessel containing the balloon;
a memory storing instructions; and
one or more processors configured to execute the instructions to
determine one or more of the inflation pressure or the injection pressure, and
cause the contrast injector to stop injection of the contrast medium based on one or more of the inflation pressure or the injection pressure.

21. The tissue treatment system of claim 20, wherein the contrast injector includes a housing to receive a syringe containing the contrast medium, and wherein the housing includes a pivotable cap having a slot sized to receive a plunger flange of the syringe.

22. The tissue treatment system of claim 21, wherein the slot is sized to receive a second plunger flange of a second syringe sized differently than the plunger flange of the syringe.

23. The tissue treatment system of claim 20 further comprising:
a tissue treatment catheter, an ultrasound transducer contained within the balloon; and
an energy delivery unit to energize the ultrasound transducer to emit acoustic energy through the inflation fluid to an ablation target.

24. The tissue treatment system of claim 20 further comprising:
a contrast reservoir to store contrast medium, the contrast reservoir located in a fluid transfer cartridge.

25. The tissue treatment system of claim 24, wherein the contrast reservoir is located proximate to a first syringe barrel.

26. The tissue treatment system of claim 25, wherein the contrast reservoir is located between the first syringe barrel and a second syringe barrel.

27. The tissue treatment system of claim 24, wherein the contrast reservoir is located behind a handle front plate.

28. A fluid transfer cartridge, comprising:
a cartridge shell defining a cartridge cavity, wherein the cartridge shell includes one or more stabilizing prongs;
a syringe barrel disposed within the cartridge cavity; and
a syringe piston disposed within the syringe barrel, wherein the syringe piston includes a stopper and a shaft extending longitudinally from the stopper to a shaft end, and wherein the shaft includes a plurality of piston notches each of which receives at least one of the one or more stabilizing prongs.

29. The fluid transfer cartridge of claim 28, wherein two of the stabilizing prongs face each other and the shaft extends through a gap separating the two stabilizing prongs.

30. The fluid transfer cartridge of claim 29, wherein the shaft includes an external thread, and further comprising a gear mounted on the cartridge shell, wherein the gear includes an internal thread engaging the external thread.

31. The fluid transfer cartridge of claim 30, wherein the one or more stabilizing prongs have longitudinal widths greater than a pitch of the external thread.

32. The fluid transfer cartridge of claim 31, wherein the longitudinal widths of the one or more stabilizing prongs are at least 1.5 times the pitch of the external thread.

33. A fluid transfer cartridge, comprising:
a cartridge shell defining a cartridge cavity, wherein the cartridge shell includes a plurality of stabilizing prongs separated by a gap;
a syringe barrel disposed within the cartridge cavity; and
a syringe piston disposed within the syringe barrel, wherein the syringe piston includes a stopper and a shaft extending longitudinally from the stopper through the gap to a shaft end.

34. The fluid transfer cartridge of claim 33, wherein the plurality of stabilizing prongs are located in a plurality of piston notches of the shaft.

35. The fluid transfer cartridge of claim 34, wherein the shaft includes an external thread, and further comprising a gear mounted on the cartridge shell, wherein the gear includes an internal thread engaging the external thread.

36. The fluid transfer cartridge of claim 35, wherein the plurality of stabilizing prongs have longitudinal widths greater than a pitch of the external thread.

37. The fluid transfer cartridge of claim 36, wherein the longitudinal widths of the plurality of stabilizing prongs are at least 1.5 times the pitch of the external thread.

38. A fluid transfer cartridge, comprising:
a cartridge shell defining a cartridge cavity between a front shell portion and a rear shell portion, wherein the rear shell portion includes a sidewall extending longitudinally from a rear base plate to a rear face, wherein a plurality of latch keepers extend through the sidewall, and wherein a plurality of latch guides are formed along an edge between the sidewall and the rear face.

39. The fluid transfer cartridge of claim 38, wherein each latch guide of the plurality of latch guides is longitudinally aligned with a respective one of the plurality of latch keepers.

40. The fluid transfer cartridge of claim 38, wherein each latch guide of the plurality of latch guides includes a bevel on the edge.

41. The fluid transfer cartridge of claim 40, wherein a length of the bevel is greater than a width of the respective one of the plurality of latch keepers.

42. A fluid transfer cartridge, comprising:
a cartridge shell defining a cartridge cavity between a front shell portion and a rear shell portion, wherein the rear shell portion includes a label recess in a rear face, and wherein the label recess has a depth of 0.01 to 0.02 inch.

43. The fluid transfer cartridge of claim 42, wherein the depth is 0.015 inch.

44. The fluid transfer cartridge of claim 42, further comprising a label mounted in the label recess, wherein the label has a thickness that is smaller than the depth of the label recess.

45. A fluid transfer cartridge, comprising:
a cartridge shell defining a cartridge cavity between a front shell portion and a rear shell portion, wherein the rear shell portion includes a sidewall extending longitudinally from a rear base plate to a rear face, and wherein a plurality of latch holes extend through the sidewall; and
a shell plate contained within the cartridge cavity between the front shell portion and the rear shell portion, wherein the shell plate includes a plurality of lateral projections extending into the plurality of latch holes such that a plurality of latch keepers are defined between the plurality of lateral projections and the rear face.

46. A fluid transfer cartridge, comprising:
a cartridge shell defining a cartridge cavity between a front face and a rear face, wherein the cartridge shell includes a handle extending from the front face over an opening, and wherein the handle includes a detachable front plate.

47. The fluid transfer cartridge of claim 46, wherein the handle includes the detachable front plate mounted on a rear handle portion, and wherein a front surface of the detachable front plate is entirely smooth.

48. The fluid transfer cartridge of claim 47, wherein a rear surface of the rear handle portion is entirely smooth.

49. The fluid transfer cartridge of claim 48, wherein an anterior surface of the rear handle portion has a molding gate, and wherein the detachable front plate covers the molding gate.

50. The fluid transfer cartridge of claim 49, wherein the molding gate is located at a middle of the anterior surface.

51. A fluid transfer cartridge, comprising:
a cartridge shell defining a cartridge cavity between a front face and a rear face, wherein the front face includes a tab extending into a conduit port defined by an edge; and
a conduit routing plate engaging the front face along the edge, wherein the tab is located in a conduit slot of the conduit routing plate.

52. The fluid transfer cartridge of claim 51, wherein the front face and the conduit routing plate include respective notches at the edge, and wherein the respective notches combine to form a conduit routing port through which a fluid conduit is routed.
